SEQUENCE LISTING (47 sequences)

SEQ ID NO: 1 - protein, 538 aa, Artificial Sequence
  Description of Artificial Sequence Synthetic polypeptide
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 2 - DNA, 1614 bp, Artificial Sequence
  Description of Artificial Sequence Synthetic polynucleotide
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgac

SEQ ID NO: 3 - DNA, 1614 bp, Artificial Sequence
  Description of Artificial Sequence Synthetic polynucleotide
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgac

SEQ ID NO: 4 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - DNA, 3822 bp, Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 6 - DNA, 3822 bp, Artificial Sequence
  Description of Artificial Sequence Synthetic polynucleotide
atgttcgtgtttctggtgctgctgcctctggtgagctcccagtgcgtgaacctgaccacaaggacccagctcccccctgcctataccaattccttcacacggggcgtgtactatcccgacaaggtgtttagatctagcgtgctgcactccacacaggatctgtttctgcctttcttttctaacgtgacctggttccacgccatccatgtgagcggcaccaatggcacaaagcggttcgacaatccagtgctgccctttaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatccattcctgggcgtgtactatcacaagaacaataagtcttggatggagagcgagtttcgcgtgtattcctctgccaacaattgcacatttgagtacgtgtcccagcccttcctgatggacctggagggcaagcagggcaatttcaagaacctgagggagttcgtgtttaagaatatcgatggctacttcaagatctactccaagcacaccccaatcaacctggtgcgcgacctgccacagggcttctctgccctggagccactggtggatctgcccatcggcatcaacatcacccggtttcagacactgctggccctgcacagaagctacctgacaccaggcgacagctcctctggatggaccgccggggccgccgcctactatgtgggctatctgcagcccaggaccttcctgctgaagtacaacgagaatggcaccatcacagacgcagtggattgcgccctggaccccctgtctgagaccaagtgtacactgaagagctttaccgtggagaagggcatctatcagacaagcaatttcagggtgcagcctaccgagtccatcgtgcgctttcccaatatcacaaacctgtgcccttttggcgaggtgttcaacgcaaccaggttcgccagcgtgtacgcatggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgcagactacaattataagctgcctgacgatttcaccggctgcgtgatcgcctggaactctaacaatctggatagcaaagtgggcggcaactacaattatctgtaccggctgtttagaaagtctaatctgaagccattcgagagggacatctccacagagatctaccaggccggctctaccccctgcaatggcgtggagggctttaactgttatttccctctgcagagctacggcttccagccaacaaacggcgtgggctatcagccctaccgcgtggtggtgctgtcttttgagctgctgcacgcacctgcaacagtgtgcggaccaaagaagagcaccaatctggtgaagaacaagtgcgtgaacttcaacttcaacggcctgaccggcacaggcgtgctgaccgagtccaacaagaagttcctgccttttcagcagttcggcagggacatcgcagataccacagacgccgtgcgcgaccctcagaccctggagatcctggacatcacaccatgctccttcggcggcgtgtctgtgatcacaccaggcaccaatacaagcaaccaggtggccgtgctgtatcaggacgtgaattgtaccgaggtgcccgtggccatccacgcagatcagctcacccctacatggcgggtgtactctaccggcagcaacgtgttccagacaagagccggctgcctgatcggagccgagcatgtgaacaatagctatgagtgcgacatccctatcggagccggcatctgtgcctcctaccagacccagacaaactccccacggagagcccggtctgtggccagccagtccatcatcgcctataccatgagcctgggggccgagaacagcgtggcctactccaacaattctatcgccatccctaccaacttcacaatctccgtgaccacagagatcctgccagtgagcatgaccaagacatccgtggactgcacaatgtatatctgtggcgattccaccgagtgctctaacctgctgctgcagtacggctctttttgtacccagctcaacagagccctgacaggcatcgccgtggagcaggacaagaacacacaggaggtgttcgcccaggtgaagcagatctacaagaccccacccatcaaggactttggcggcttcaacttcagccagatcctgcccgatcctagcaagccatccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggggccggggccgccctgcagatcccctttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctcagcagcaacttcggggccatcagcagcgtgctgaacgacatcctgagccggctggacaaggtggaggcagaggtgcagatcgaccggctgatcacaggcagactgcagtccctgcagacctacgtgacacagcagctcatcagggccgccgagatcagggcctctgccaatctggccgccaccaagatgagcgagtgcgtgctgggccagtccaagagagtggacttttgtggcaagggctatcacctgatgagcttcccacagtccgcccctcacggagtggtgtttctgcatgtgacctacgtgccagcccaggagaagaacttcaccacagcccccgcaatctgccacgatggcaaggcacactttccccgggagggcgtgttcgtgagcaacggcacccactggtttgtgacacagcgcaatttctacgagccacagatcatcaccacagacaatacattcgtgtccggcaactgtgacgtggtcatcggcatcgtgaacaataccgtgtatgatcctctgcagccagagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacaagctgctgttcctgcctgaagggctgctgttcttgtggctcctgctgtaagtttgatgaggacgatagcgagcctgtgctgaagggcgtgaagctgcactacacctga

SEQ ID NO: 7 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 8 - DNA, 3768 bp, Severe acute respiratory syndrome-related coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 9 - DNA, 3768 bp, Artificial Sequence
  Description of Artificial Sequence Synthetic polynucleotide
atgttcatctttctgctgttcctgaccctgacaagcggctccgacctggataggtgcaccacatttgacgatgtgcaggcccccaactacacacagcacaccagctccatgaggggcgtgtactatcctgatgagatcttccgctctgacacactgtacctgacccaggacctgttcctgcctttttatagcaatgtgacaggcttccacaccatcaatcacacatttggcaaccccgtgatccctttcaaggatggcatctactttgccgccaccgagaagtctaacgtggtgcggggatgggtgttcggcagcacaatgaacaataagtctcagagcgtgatcatcatcaacaatagcaccaacgtggtcatcagagcctgcaattttgagctgtgcgacaaccccttctttgccgtgtccaagcctatgggcacccagacacacaccatgatctttgataatgccttcaactgtacctttgagtacatcagcgatgccttttccctggacgtgtctgagaagtccggcaacttcaagcacctgagggagttcgtgtttaagaataaggacggcttcctgtacgtgtataagggctatcagcccatcgatgtggtgcgcgacctgccttccggcttcaacaccctgaagccaatctttaagctgcccctgggcatcaatatcaccaacttcagggccatcctgacagcctttagcccagcacaggacatctggggcaccagcgccgccgcctacttcgtgggctatctgaagcccaccaccttcatgctgaagtacgatgagaacggcacaatcaccgacgccgtggattgcagccagaatccactggccgagctgaagtgttccgtgaagtctttcgagatcgacaagggcatctatcagacctccaactttagggtggtgccatctggcgatgtggtgcgcttcccaaatatcaccaacctgtgccccttcggcgaggtgtttaatgccacaaagttccccagcgtgtacgcctgggagcgcaagaagatcagcaactgcgtggccgactactccgtgctgtataatagcaccttcttcagcaccttcaagtgctacggcgtgagcgccaccaagctgaatgacctgtgcttctctaacgtgtatgccgatagctttgtggtgaagggcgacgatgtgaggcagatcgcacctggacagaccggcgtgatcgcagactacaactataagctgccagacgatttcatgggctgcgtgctggcctggaatacacgcaacatcgatgccacatccaccggcaactacaattataagtaccggtatctgagacacggcaagctgcggcccttcgagagagacatctccaatgtgccattttctccagatggcaagccatgcaccccacctgccctgaattgttactggcctctgaacgactacggcttctataccacaaccggcatcggctaccagccttatagggtggtggtgctgtcctttgagctgctgaacgcacctgcaaccgtgtgcggaccaaagctgtctacagatctgatcaagaatcagtgcgtgaacttcaacttcaacggcctgacaggcaccggcgtgctgaccccttctagcaagcggttccagccatttcagcagttcggcagagacgtgagcgatttcaccgactccgtgcgcgacccaaagacatccgagatcctggacatcagcccctgctcctttggcggcgtgtctgtgatcacacctggcaccaacgcctcctctgaggtggccgtgctgtaccaggatgtgaattgtaccgacgtgagcacagcaatccacgcagaccagctcaccccagcatggcggatctattccaccggcaacaacgtgttccagacacaggcaggatgcctgatcggagccgagcatgtggatacaagctacgagtgcgacatccccatcggagccggcatctgtgcctcttatcacaccgtgagcctgctgagatccacatctcagaagtctatcgtggcctacaccatgagcctgggggccgatagctccatcgcctattccaacaataccatcgccatcccaacaaacttcagcatctccatcacaaccgaagtgatgcccgtgtccatggccaagacctctgtggactgcaacatgtacatctgtggcgatagcacagagtgcgccaatctgctgctgcagtatggctccttttgtacccagctcaaccgggccctgtctggaatcgccgccgagcaggacaggaatacacgcgaggtgttcgcccaggtgaagcagatgtacaagacacctaccctgaagtattttggcggcttcaacttttctcagatcctgcctgatccactgaagccaaccaagcggagcttcatcgaggacctgctgtttaataaggtgacactggccgatgccggcttcatgaagcagtacggcgagtgcctgggcgacatcaacgccagagacctgatctgtgcccagaagtttaatggcctgaccgtgctgccacccctgctgacagacgatatgatcgcagcatataccgccgccctggtgtccggcacagccaccgccggctggaccttcggggccggggccgccctgcagatccctttcgccatgcagatggcctaccggtttaacggcatcggcgtgacccagaatgtgctgtatgagaaccagaagcagatcgccaatcagtttaacaaggccatcagccagatccaggagtccctgacaaccacatctaccgccctgggcaagctgcaggacgtggtgaatcagaacgcccaggccctgaatacactggtgaagcagctcagcagcaacttcggggccatcagcagcgtgctgaacgacatcctgagccggctggacaaggtggaggcagaggtgcagatcgataggctgatcaccggcagactgcagtctctgcagacatacgtgacccagcagctcatcagggccgccgagatcagagccagcgccaacctggccgccacaaagatgtccgagtgcgtgctgggccagtctaagagggtggacttctgtggcaagggctaccacctgatgtcctttccacaggccgcccctcacggagtggtgttcctgcatgtgacctatgtgccttctcaggagcgcaactttaccacagccccagcaatctgccacgagggcaaggcatacttcccccgggagggcgtgttcgtgtttaacggcacctcctggtttatcacacagagaaatttcttttcccctcagatcatcaccacagacaataccttcgtgagcggcaactgtgacgtggtcatcggcatcatcaacaatacagtgtacgatcctctgcagccagagctggacagcttcaaggaggagctggataagtacttcaagaaccacacctcccccgacgtggatctgggcgacatcagcggcatcaatgcctccgtggtgaacatccagaaggagatcgacagactgaatgaggtggccaagaatctgaacgagtccctgatcgatctgcaggagctgggcaagtacgagcagtatatcaagtggccatggtacgtgtggctgggcttcatcgccggcctgatcgccatcgtgatggtgaccatcctgctgtgctgtatgacatcttgctgtagctgcctgaagggagcctgctcctgtggctcttgctgtaagtttgacgaggacgatagcgagcccgtgctgaagggcgtgaagctgcactatacctga

SEQ ID NO: 10 - protein, 1353 aa, Middle East respiratory syndrome-related coronavirus
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFQKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 11 - DNA, 4062 bp, Middle East respiratory syndrome-related coronavirus
atgatacactcagtgtttctactgatgttcttgttaacacctacagaaagttacgttgatgtagggccagattctgttaagtctgcttgtattgaggttgatatacaacagactttctttgataaaacttggcctaggccaattgatgtttctaaggctgacggtattatataccctcaaggccgtacatattctaacataactatcacttatcaaggtctttttccctatcagggagaccatggtgatatgtatgtttactctgcaggacatgctacaggcacaactccacaaaagttgtttgtagctaactattctcaggacgtcaaacagtttgctaatgggtttgtcgtccgtataggagcagctgccaattccactggcactgttattattagcccatctaccagcgctactatacgaaaaatttaccctgcttttatgctgggttcttcagttggtaatttctcagatggtaaaatgggccgcttcttcaatcatactctagttcttttgcccgatggatgtggcactttacttagagctttttattgtattctagagcctcgctctggaaatcattgtcctgctggcaattcctatacttcttttgccacttatcacactcctgcaacagattgttctgatggcaattacaatcgtaatgccagtctgaactcttttaaggagtattttaatttacgtaactgcacctttatgtacacttataacattaccgaagatgagattttagagtggtttggcattacacaaactgctcaaggtgttcacctcttctcatctcggtatgttgatttgtacggcggcaatatgtttcaatttgccaccttgcctgtttatgatactattaagtattattctatcattcctcacagtattcgttctatccaaagtgatagaaaagcttgggctgccttctacgtatataaacttcaaccgttaactttcctgttggatttttctgttgatggttatatacgcagagctatagactgtggttttaatgatttgtcacaactccactgctcatatgaatccttcgatgttgaatctggagtttattcagtttcgtctttcgaagcaaaaccttctggctcagttgtggaacaggctgaaggtgttgaatgtgatttttcacctcttctgtctggcacacctcctcaggtttataatttcaagcgtttggtttttaccaattgcaattataatcttaccaaattgctttcacttttttctgtgaatgattttacttgtagtcaaatatctccagcagcaattgctagcaactgttattcttcactgattttggattacttttcatacccacttagtatgaaatccgatctcagtgttagttctgctggtccaatatcccagtttaattataaacagtccttttctaatcccacatgtttgattttagcgactgttcctcataaccttactactattactaagcctcttaagtacagctatattaacaagtgctctcgtcttctttctgatgatcgtactgaagtacctcagttagtgaacgctaatcaatactcaccctgtgtatccattgtcccatccactgtgtgggaagacggtgattattataggaaacaactatctccacttgaaggtggtggctggcttgttgctagtggctcaactgttgccatgactgagcaattacagatgggctttggtattacagttcaatatggtacagacaccaatagtgtttgccccaagcttgaatttgctaatgacacaaaaattgcctctcaattaggcaattgcgtggaatattccctctatggtgtttcgggccgtggtgtttttcagaattgcacagctgtaggtgttcgacagcagcgctttgtttatgatgcgtaccagaatttagttggctattattctgatgatggcaactactactgtttgcgtgcttgtgttagtgttcctgtttctgtcatctatgataaagaaactaaaacccacgctactctatttggtagtgttgcatgtgaacacatttcttctaccatgtctcaatactcccgttctacgcgatcaatgcttaaacggcgagattctacatatggcccccttcagacacctgttggttgtgtcctaggacttgttaattcctctttgttcgtagaggactgcaagttgcctcttggtcaatctctctgtgctcttcctgacacacctagtactctcacacctcgcagtgtgcgctctgttccaggtgaaatgcgcttggcatccattgcttttaatcatcctattcaggttgatcaacttaatagtagttattttaaattaagtatacccactaatttttcctttggtgtgactcaggagtacattcagacaaccattcagaaagttactgttgattgtaaacagtacgtttgcaatggtttccagaagtgtgagcaattactgcgcgagtatggccagttttgttccaaaataaaccaggctctccatggtgccaatttacgccaggatgattctgtacgtaatttgtttgcgagcgtgaaaagctctcaatcatctcctatcataccaggttttggaggtgactttaatttgacacttctagaacctgtttctatatctactggcagtcgtagtgcacgtagtgctattgaggatttgctatttgacaaagtcactatagctgatcctggttatatgcaaggttacgatgattgcatgcagcaaggtccagcatcagctcgtgatcttatttgtgctcaatatgtggctggttacaaagtattacctcctcttatggatgttaatatggaagccgcgtatacttcatctttgcttggcagcatagcaggtgttggctggactgctggcttatcctcctttgctgctattccatttgcacagagtatcttttataggttaaacggtgttggcattactcaacaggttctttcagagaaccaaaagcttattgccaataagtttaatcaggctctgggagctatgcaaacaggcttcactacaactaatgaagcttttcagaaggttcaggatgctgtgaacaacaatgcacaggctctatccaaattagctagcgagctatctaatacttttggtgctatttccgcctctattggagacatcatacaacgtcttgatgttctcgaacaggacgcccaaatagacagacttattaatggccgtttgacaacactaaatgcttttgttgcacagcagcttgttcgttccgaatcagctgctctttccgctcaattggctaaagataaagtcaatgagtgtgtcaaggcacaatccaagcgttctggattttgcggtcaaggcacacatatagtgtcctttgttgtaaatgcccctaatggcctttacttcatgcatgttggttattaccctagcaaccacattgaggttgtttctgcttatggtctttgcgatgcagctaaccctactaattgtatagcccctgttaatggctactttattaaaactaataacactaggattgttgatgagtggtcatatactggctcgtccttctatgcacctgagcccattacctcccttaatactaagtatgttgcaccacaggtgacataccaaaacatttctactaacctccctcctcctcttctcggcaattccaccgggattgacttccaagatgagttggatgagtttttcaaaaatgttagcaccagtatacctaattttggttccctaacacagattaatactacattactcgatcttacctacgagatgttgtctcttcaacaagttgttaaagcccttaatgagtcttacatagaccttaaagagcttggcaattatacttattacaacaaatggccgtggtacatttggcttggtttcattgctgggcttgttgccttagctctatgcgtcttcttcatactgtgctgcactggttgtggcacaaactgtatgggaaaacttaagtgtaatcgttgttgtgatagatacgaggaatacgacctcgagccgcataaggttcatgttcactaa

SEQ ID NO: 12 - DNA, 4062 bp, Artificial Sequence
  Description of Artificial Sequence Synthetic polynucleotide
atgatccacagcgtgttcctgctgatgtttctgctgacacctaccgagtcctacgtggatgtgggcccagactctgtgaagagcgcctgcatcgaggtggacatccagcagacattctttgacaagacctggcccagacccatcgacgtgagcaaggcagacggaatcatctacccacagggacgcacatatagcaacatcacaatcacctaccagggcctgttcccttatcagggcgaccacggcgatatgtacgtgtatagcgccggccacgcaaccggcaccacaccacagaagctgtttgtggccaattattcccaggacgtgaagcagttcgccaacggatttgtggtgcggatcggggccgccgccaacagcacaggcaccgtgatcatctctcccagcacatccgccaccatcagaaagatctaccctgcctttatgctgggcagctccgtgggcaacttctccgatggcaagatgggcaggttctttaatcacacactggtgctgctgccagacggatgcggcaccctgctgagggccttctactgtatcctggagccccgctctggaaatcactgccctgccggcaactcctacacctcttttgccacatatcacacccctgccacagactgttccgatggcaattataaccggaatgccagcctgaactccttcaaggagtactttaatctgagaaactgcaccttcatgtacacatataatatcaccgaggatgagatcctggagtggttcggcatcacacagaccgcccagggcgtgcacctgttttctagcagatacgtggatctgtatggcggcaacatgttccagtttgccacactgccagtgtatgacaccatcaagtactatagcatcatcccccactctatccggagcatccagtccgacagaaaggcctgggccgccttctacgtgtataagctgcagcccctgaccttcctgctggatttttccgtggacggctacatccggagagccatcgattgcggctttaacgacctgtctcagctccactgttcttatgagagcttcgatgtggagtctggcgtgtacagcgtgtcctcttttgaggccaagccatctggcagcgtggtggagcaggcagagggagtggagtgcgacttctccccactgctgtctggcacaccacctcaggtgtataatttcaagaggctggtgtttacaaactgtaattacaacctgaccaagctgctgtccctgttctctgtgaacgactttacctgcagccagatctcccctgccgccatcgcctccaattgttatagctccctgatcctggattacttctcttatcccctgtctatgaagagcgacctgtccgtgtctagcgccggccctatcagccagtttaattacaagcagtccttctctaaccccacatgcctgatcctggccaccgtgcctcacaacctgaccacaatcacaaagccactgaagtactcctatatcaataagtgcagcaggctgctgtccgacgatcgcaccgaggtgcctcagctcgtgaacgccaaccagtactctccatgcgtgagcatcgtgccatccaccgtgtgggaggacggcgattactatagaaagcagctcagcccactggagggaggaggatggctggtggccagcggctccacagtggccatgaccgagcagctccagatgggcttcggcatcacagtgcagtacggcacagataccaatagcgtgtgccccaagctggagtttgccaacgacaccaagatcgcctcccagctcggcaattgcgtggagtactccctgtatggcgtgtctggcagaggcgtgttccagaactgtacagccgtgggcgtgcggcagcagcggttcgtgtacgatgcctatcagaacctggtgggctactatagcgacgatggcaattactattgcctgagggcatgcgtgagcgtgcccgtgagcgtgatctacgacaaggagacaaagacccacgccaccctgttcggctccgtggcctgcgagcacatctcctctacaatgtctcagtattctaggagcacccgctctatgctgaagaggcgcgacagcacatacggaccactgcagacccctgtgggatgcgtgctgggcctggtgaacagcagcctgtttgtggaggattgcaagctgccactgggccagtctctgtgcgcactgccagacacccccagcacactgaccccacggtctgtgagaagcgtgcccggagagatgagactggccagcatcgccttcaatcaccctatccaggtggatcagctcaacagcagctactttaagctgagcatcccaacaaacttctcctttggcgtgacccaggagtatatccagaccacaatccagaaggtgaccgtggactgcaagcagtacgtgtgcaatggcttccagaagtgcgagcagctcctgagggagtatggccagttttgttccaagatcaatcaggccctgcacggagccaacctgaggcaggacgattccgtgagaaacctgttcgcctctgtgaagtcctctcagagctcccctatcatcccaggcttcggcggcgacttcaacctgaccctgctggagcccgtgtccatctctaccggcagcaggtccgcccgcagcgccatcgaggatctgctgtttgacaaggtgaccatcgccgacccaggctacatgcagggctatgacgattgcatgcagcagggaccagcctccgcccgcgatctgatctgtgcccagtacgtggccggctataaggtgctgccacccctgatggacgtgaacatggaggccgcctatacatctagcctgctgggcagcatcgcaggagtgggatggaccgccggcctgtcctctttcgccgcaatcccttttgcccagtctatcttctaccggctgaacggcgtgggcatcacacagcaggtgctgagcgagaatcagaagctgatcgccaataagttcaaccaggccctgggggccatgcagaccggctttaccacaaccaacgaggccttccagaaggtgcaggatgccgtgaacaataacgcacaggccctgtccaagctggcctccgagctgtctaataccttcggggccatcagcgccagcatcggcgacatcatccagcgcctggacgtgctggagcaggatgcccagatcgacaggctgatcaatggccgcctgacaaccctgaacgcctttgtggcacagcagctcgtgcggagcgagtctgccgccctgagcgcccagctcgccaaggacaaggtgaacgagtgcgtgaaggcccagagcaagcggtccggcttttgtggccagggcacccacatcgtgtccttcgtggtgaatgcccctaacggcctgtactttatgcatgtgggctactatccaagcaaccacatcgaggtggtgtccgcctatggcctgtgcgatgccgccaatcctacaaactgtatcgccccagtgaatggctacttcatcaagaccaataacacacggatcgtggacgagtggtcctacaccggcagctccttttatgcccccgagcctatcacatctctgaacaccaagtacgtggccccacaggtgacatatcagaatatcagcaccaacctgcctccacccctgctgggcaattccaccggcatcgacttccaggatgagctggacgagttctttaagaatgtgagcacatccatccccaactttggcagcctgacccagatcaacacaaccctgctggatctgacatacgagatgctgtctctgcagcaggtggtgaaggccctgaatgagagctacatcgacctgaaggagctgggcaattatacctactataacaagtggccttggtacatctggctgggcttcatcgcaggcctggtggccctggccctgtgcgtgttctttatcctgtgctgtacaggctgcggcaccaattgtatgggcaagctgaagtgtaaccggtgctgtgatagatacgaggagtatgacctggagccacacaaggtgcatgtgcactga

SEQ ID NO: 13 - protein, 1273 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - DNA, 3822 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 15 - DNA, 3822 bp, Artificial Sequence
  Description of Artificial Sequence Synthetic polynucleotide
atgttcgtgtttctggtgctgctgcctctggtgagctcccagtgcgtgaacctgaccacaaggacccagctcccccctgcctataccaattccttcacacggggcgtgtactatccagacaaggtgtttagatctagcgtgctgcactccacacaggatctgtttctgcccttcttttctaacgtgacctggttccacgccatccatgtgagcggcaccaatggcacaaagcggttcgacaatcctgtgctgcccttcaacgatggcgtgtacttcgcctccaccgagaagtctaacatcatcagaggctggatctttggcaccacactggacagcaagacacagtccctgctgatcgtgaacaatgccaccaacgtggtcatcaaggtgtgcgagttccagttttgtaatgatcctttcctgggcgtgtactatcacaagaacaataagtcttggatggagagcgagtttcgcgtgtattcctctgccaacaattgcacatttgagtacgtgtcccagccattcctgatggacctggagggcaagcagggcaatttcaagaacctgagggagttcgtgtttaagaatatcgatggctacttcaagatctactccaagcacacccctatcaacctggtgcgcgacctgccacagggcttctctgccctggagcctctggtggatctgccaatcggcatcaacatcacccggtttcagacactgctggccctgcacagaagctacctgacacctggcgacagctcctctggatggaccgccggggccgccgcctactatgtgggctatctgcagccaaggaccttcctgctgaagtacaacgagaatggcaccatcacagacgcagtggattgcgccctggaccccctgtctgagaccaagtgtacactgaagagctttaccgtggagaagggcatctatcagacaagcaatttcagggtgcagcccaccgagtccatcgtgcgctttccaaatatcacaaacctgtgcccctttggcgaggtgttcaacgcaaccaggttcgccagcgtgtacgcatggaataggaagcgcatctccaactgcgtggccgactattctgtgctgtacaacagcgcctccttctctacctttaagtgctatggcgtgagccccacaaagctgaatgacctgtgctttaccaacgtgtacgccgattccttcgtgatcaggggcgacgaggtgcgccagatcgcaccaggacagacaggcaagatcgccgactacaattataagctgcccgacgatttcaccggctgcgtgatcgcctggaactctaacaatctggatagcaaagtgggcggcaactacaattatctgtaccggctgtttagaaagtctaatctgaagcctttcgagagggacatctccacagagatctaccaggccggctctaccccatgcaatggcgtggagggctttaactgttatttccccctgcagagctacggcttccagcctacaaacggcgtgggctatcagccataccgcgtggtggtgctgtcttttgagctgctgcacgcaccagcaacagtgtgcggacctaagaagagcaccaatctggtgaagaacaagtgcgtgaacttcaacttcaacggcctgaccggcacaggcgtgctgaccgagtccaacaagaagttcctgccctttcagcagttcggcagggacatcgcagataccacagacgccgtgcgcgacccccagaccctggagatcctggacatcacaccttgctccttcggcggcgtgtctgtgatcacacctggcaccaatacaagcaaccaggtggccgtgctgtatcaggacgtgaattgtaccgaggtgccagtggccatccacgccgatcagctcacccccacatggcgggtgtactctaccggcagcaacgtgttccagacaagagccggctgcctgatcggagccgagcatgtgaacaatagctatgagtgcgacatccccatcggagccggcatctgtgcctcctaccagacccagacaaactcccctcggagagcccggtctgtggccagccagtccatcatcgcctataccatgagcctgggggccgagaacagcgtggcctactccaacaattctatcgccatccccaccaacttcacaatctccgtgaccacagagatcctgcctgtgagcatgaccaagacatccgtggactgcacaatgtatatctgtggcgattccaccgagtgctctaacctgctgctgcagtacggctctttttgtacccagctcaacagagccctgacaggcatcgccgtggagcaggacaagaacacacaggaggtgttcgcccaggtgaagcagatctacaagaccccacccatcaaggactttggcggcttcaacttcagccagatcctgccagatcccagcaagccttccaagcggtcttttatcgaggacctgctgttcaacaaggtgaccctggccgatgccggcttcatcaagcagtatggcgattgcctgggcgacatcgccgccagagacctgatctgtgcccagaagtttaatggcctgaccgtgctgcctccactgctgacagatgagatgatcgcccagtacacatctgccctgctggccggcaccatcacaagcggatggaccttcggggccggggccgccctgcagatcccatttgccatgcagatggcctatcggttcaacggcatcggcgtgacccagaatgtgctgtacgagaaccagaagctgatcgccaatcagtttaactccgccatcggcaagatccaggactctctgagctccacagccagcgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgaccggctgatcacaggcagactgcagtccctgcagacctacgtgacacagcagctcatcagggccgccgagatcagggcctctgccaatctggccgccaccaagatgagcgagtgcgtgctgggccagtccaagagagtggacttttgtggcaagggctatcacctgatgagcttcccacagtccgccccccacggagtggtgtttctgcatgtgacctacgtgcctgcccaggagaagaacttcaccacagccccagccatctgccacgatggcaaggcccactttcccagggagggcgtgttcgtgagcaacggcacccactggtttgtgacacagcgcaatttctacgagcctcagatcatcaccacagacaatacattcgtgtccggcaactgtgacgtggtcatcggcatcgtgaacaataccgtgtatgatcccctgcagcctgagctggactcttttaaggaggagctggataagtacttcaagaatcacaccagccccgacgtggatctgggcgacatctctggcatcaatgccagcgtggtgaacatccagaaggagatcgacaggctgaacgaggtggccaagaatctgaacgagtccctgatcgatctgcaggagctgggcaagtatgagcagtacatcaagtggccatggtatatctggctgggcttcatcgccggcctgatcgccatcgtgatggtgaccatcatgctgtgctgtatgacaagctgctgttcctgcctgaagggctgctgttcttgtggctcctgctgtaagtttgatgaggacgatagcgagcccgtgctgaagggcgtgaagctgcactacacctga

SEQ ID NO: 16 - protein, 1273 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - DNA, 3822 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtagtgcaagtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 18 - DNA, 3822 bp, Artificial Sequence
  Description of Artificial Sequence Synthetic polynucleotide
atgttcgtgtttctggtgctgctgcctctggtgagctcccagtgcgtgaacctgaccacaaggacccagctcccccctgcctataccaattccttcacaaggggcgtgtactatcccgacaaggtgtttcgctctagcgtgctgcacagcacacaggatctgtttctgcctttcttttccaacgtgacctggttccacgccatccatgtgagcggcaccaatggcacaaagaggttcgacaatccagtgctgccctttaacgatggcgtgtacttcgcctctaccgagaagagcaacatcatccgcggctggatctttggcaccacactggactccaagacacagtctctgctgatcgtgaacaatgccaccaacgtggtcatcaaggtgtgcgagttccagttttgtaatgatccattcctgggcgtgtactatcacaagaacaataagagctggatggagtccgagtttcgcgtgtattcctctgccaacaattgcacatttgagtacgtgtcccagcccttcctgatggacctggagggcaagcagggcaatttcaagaacctgcgggagttcgtgtttaagaatatcgatggctacttcaagatctacagcaagcacaccccaatcaacctggtgagagacctgccacagggcttctccgccctggagccactggtggatctgcccatcggcatcaacatcaccaggtttcagacactgctggccctgcaccgcagctacctgacaccaggcgacagctcctctggatggaccgccggggccgccgcctactatgtgggctatctgcagccccggaccttcctgctgaagtacaacgagaatggcaccatcacagacgcagtggattgcgccctggaccccctgtccgagaccaagtgtacactgaagtcttttaccgtggagaagggcatctatcagacatctaatttccgggtgcagcctaccgagagcatcgtgagatttcccaatatcacaaacctgtgcccttttggcgaggtgttcaacgccaccagattcgccagcgtgtacgcctggaatcggaagagaatcagcaactgcgtggccgactattccgtgctgtacaactctgccagcttctccacctttaagtgctatggcgtgtctcccacaaagctgaatgacctgtgctttaccaacgtgtacgccgatagcttcgtgatcaggggcgacgaggtgagacagatcgcaccaggacagacaggcaagatcgcagactacaattataagctgcctgacgatttcaccggctgcgtgatcgcctggaacagcaacaatctggattccaaagtgggcggcaactacaattatctgtacaggctgtttcgcaagtccaatctgaagccattcgagcgggacatcagcacagagatctaccaggcaggctccaccccatgcaatggagtggagggctttaactgttatttccctctgcagtcttacggcttccagccaacaaacggcgtgggctatcagccctacagagtggtggtgctgtcctttgagctgctgcacgcacctgcaacagtgtgcggaccaaagaagtctaccaatctggtgaagaacaagtgcgtgaacttcaacttcaacggcctgaccggcacaggcgtgctgaccgagtccaacaagaagttcctgccttttcagcagttcggcagagacatcgccgataccacagacgccgtgagagaccctcagaccctggagatcctggacatcacaccatgctctttcggcggcgtgagcgtgatcacaccaggcaccaatacaagcaaccaggtggccgtgctgtatcaggacgtgaattgtaccgaggtgcccgtggccatccacgcagatcagctcacccctacatggagggtgtactccaccggctctaacgtgttccagacacgcgccggatgcctgatcggagccgagcatgtgaacaattcttatgagtgcgacatccctatcggagccggcatctgtgccagctaccagacccagacaaacagcccaggctccgccagctccgtggcctctcagagcatcatcgcctataccatgagcctgggggccgagaatagcgtggcctactctaacaatagcatcgccatccctaccaacttcacaatctccgtgaccacagagatcctgccagtgtccatgaccaagacatctgtggactgcacaatgtatatctgtggcgattctaccgagtgcagcaacctgctgctgcagtacggcagcttttgtacccagctcaaccgggccctgacaggaatcgcagtggagcaggacaagaacacacaggaggtgttcgcccaggtgaagcagatctacaagaccccacccatcaaggactttggcggcttcaacttcagccagatcctgcccgatccttccaagccatctaagaggagctttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcacagtacacatccgccctgctggccggcaccatcacatctggatggaccttcggggccggggccgccctgcagatcccctttgccatgcagatggcctatagattcaacggcatcggcgtgacccagaatgtgctgtacgagaaccagaagctgatcgccaatcagtttaactccgccatcggcaagatccaggactccctgtctagcacagcctctgccctgggcaagctgcaggatgtggtgaatcagaacgcccaggccctgaataccctggtgaagcagctcagcagcaacttcggggccatcagcagcgtgctgaacgacatcctgagccggctggacaaggtggaggcagaggtgcagatcgacaggctgatcacaggccgcctgcagagcctgcagacctacgtgacacagcagctcatcagggccgccgagatcagagcctccgccaatctggccgccaccaagatgtctgagtgcgtgctgggccagagcaagcgcgtggacttttgtggcaagggctatcacctgatgtccttcccacagtctgcccctcacggagtggtgtttctgcatgtgacctacgtgccagcccaggagaagaacttcaccacagcccccgcaatctgccacgatggcaaggcacactttcctcgggagggcgtgttcgtgtctaacggcacccactggtttgtgacacagagaaatttctacgagccacagatcatcaccacagacaatacattcgtgagcggcaactgtgacgtggtcatcggcatcgtgaacaataccgtgtatgatcctctgcagccagagctggactcctttaaggaggagctggataagtacttcaagaatcacacctctcccgacgtggatctgggcgacatcagcggcatcaatgcctccgtggtgaacatccagaaggagatcgacaggctgaacgaggtggccaagaatctgaacgagtccctgatcgatctgcaggagctgggcaagtatgagcagtacatcaagtggccctggtatatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgatagcgagcctgtgctgaagggcgtgaagctgcactacacctga

SEQ ID NO: 19 - protein, 1273 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - DNA, 3822 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtagtgcaagtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 21 - DNA, 3822 bp, Artificial Sequence
  Description of Artificial Sequence Synthetic polynucleotide
atgttcgtgtttctggtgctgctgcctctggtgagctcccagtgcgtgaacctgaccacaaggacccagctcccccctgcctataccaattccttcacaaggggcgtgtactatccagacaaggtgtttcgctctagcgtgctgcacagcacacaggatctgtttctgcccttcttttccaacgtgacctggttccacgccatccatgtgagcggcaccaatggcacaaagaggttcgacaatcctgtgctgcccttcaacgatggcgtgtacttcgcctctaccgagaagagcaacatcatccgcggctggatctttggcaccacactggactccaagacacagtctctgctgatcgtgaacaatgccaccaacgtggtcatcaaggtgtgcgagttccagttttgtaatgatcctttcctgggcgtgtactatcacaagaacaataagagctggatggagtccgagtttcgcgtgtattcctctgccaacaattgcacatttgagtacgtgtcccagccattcctgatggacctggagggcaagcagggcaatttcaagaacctgcgggagttcgtgtttaagaatatcgatggctacttcaagatctacagcaagcacacccctatcaacctggtgagagacctgccacagggcttctccgccctggagcctctggtggatctgccaatcggcatcaacatcaccaggtttcagacactgctggccctgcaccgcagctacctgacacctggcgacagctcctctggatggaccgccggggccgccgcctactatgtgggctatctgcagccacggaccttcctgctgaagtacaacgagaatggcaccatcacagacgcagtggattgcgccctggaccccctgtccgagaccaagtgtacactgaagtcttttaccgtggagaagggcatctatcagacatctaatttccgggtgcagcccaccgagagcatcgtgagatttccaaatatcacaaacctgtgcccctttggcgaggtgttcaacgccaccagattcgccagcgtgtacgcctggaatcggaagagaatcagcaactgcgtggccgactattccgtgctgtacaactctgccagcttctccacctttaagtgctatggcgtgtctcccacaaagctgaatgacctgtgctttaccaacgtgtacgccgatagcttcgtgatcaggggcgacgaggtgagacagatcgcaccaggccagacaggcaagatcgccgactacaattataagctgcccgacgatttcaccggctgcgtgatcgcctggaacagcaacaatctggattccaaagtgggcggcaactacaattatctgtacaggctgtttcgcaagtccaatctgaagcctttcgagcgggacatcagcacagagatctaccaggccggctccaccccatgcaatggcgtggagggctttaactgttatttccccctgcagtcttacggcttccagcctacaaacggcgtgggctatcagccatacagagtggtggtgctgtcctttgagctgctgcacgcaccagcaacagtgtgcggacctaagaagtctaccaatctggtgaagaacaagtgcgtgaacttcaacttcaacggcctgaccggcacaggcgtgctgaccgagtccaacaagaagttcctgccctttcagcagttcggcagagacatcgccgataccacagacgccgtgagagacccccagaccctggagatcctggacatcacaccttgctctttcggcggcgtgagcgtgatcacacctggcaccaatacaagcaaccaggtggccgtgctgtatcaggacgtgaattgtaccgaggtgccagtggccatccacgccgatcagctcacccccacatggagggtgtactccaccggctctaacgtgttccagacacgcgccggatgcctgatcggagccgagcatgtgaacaattcttatgagtgcgacatccccatcggagccggcatctgtgccagctaccagacccagacaaacagccctggctccgccagctccgtggcctctcagagcatcatcgcctataccatgagcctgggggccgagaatagcgtggcctactctaacaatagcatcgccatccccaccaacttcacaatctccgtgaccacagagatcctgcccgtgagcatgaccaagacatctgtggactgcacaatgtatatctgtggcgattctaccgagtgcagcaacctgctgctgcagtacggcagcttttgtacccagctcaaccgggccctgacaggaatcgcagtggagcaggacaagaacacacaggaggtgttcgcccaggtgaagcagatctacaagaccccacccatcaaggactttggcggcttcaacttcagccagatcctgccagatccctccaagccttctaagaggagctttatcgaggacctgctgttcaacaaggtgaccctggccgatgccggcttcatcaagcagtatggcgattgcctgggcgacatcgcagcccgcgacctgatctgtgcccagaagtttaatggcctgaccgtgctgcctccactgctgacagatgagatgatcgcacagtacacatccgccctgctggccggcaccatcacatctggatggaccttcggggccggggccgccctgcagatcccatttgccatgcagatggcctatagattcaacggcatcggcgtgacccagaatgtgctgtacgagaaccagaagctgatcgccaatcagtttaactccgccatcggcaagatccaggactccctgtctagcacagcctctgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgacaggctgatcacaggccgcctgcagagcctgcagacctacgtgacacagcagctcatcagggccgccgagatcagagcctccgccaatctggccgccaccaagatgtctgagtgcgtgctgggccagagcaagcgcgtggacttttgtggcaagggctatcacctgatgtccttcccacagtctgccccccacggagtggtgtttctgcatgtgacctacgtgcctgcccaggagaagaacttcaccacagccccagccatctgccacgatggcaaggcacactttccccgggagggcgtgttcgtgtctaacggcacccactggtttgtgacacagagaaatttctacgagcctcagatcatcaccacagacaatacattcgtgagcggcaactgtgacgtggtcatcggcatcgtgaacaataccgtgtatgatcccctgcagcctgagctggactcctttaaggaggagctggataagtacttcaagaatcacacctctcccgacgtggatctgggcgacatcagcggcatcaatgcctccgtggtgaacatccagaaggagatcgacaggctgaacgaggtggccaagaatctgaacgagtccctgatcgatctgcaggagctgggcaagtatgagcagtacatcaagtggccatggtatatctggctgggcttcatcgccggcctgatcgccatcgtgatggtgaccatcatgctgtgctgtatgacatcctgctgttcttgcctgaagggctgctgtagctgtggctcctgctgtaagtttgatgaggacgatagcgagcccgtgctgaagggcgtgaagctgcactacacctga

SEQ ID NO: 22 - protein, 1255 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 23 - DNA, 3768 bp, Artificial Sequence
  Description of Artificial Sequence Synthetic polynucleotide
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtagtgcaagtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatttttctttatcatagggttaatcattggactattcttggttctccgagttggtatccatctttgcattaaattaaagcacaccaagaaaagacagatttatacagacatagagatgaaccgacttggaaagtaa

SEQ ID NO: 24 - DNA, 3768 bp, Artificial Sequence
  Description of Artificial Sequence Synthetic polynucleotide
atgttcgtgttcctggtgctgctgcctctggtgagctcccagtgcgtgaacctgaccacaaggacccagctcccccctgcctataccaattcctttacaaggggcgtgtactatccagacaaggtgttccgctctagcgtgctgcactctacacaggatctgttcctgcccttctttagcaacgtgacctggtttcacgccatccatgtgagcggcaccaatggcacaaagcggtttgacaatcctgtgctgccattcaacgatggcgtgtactttgcctccaccgagaagtctaacatcatcagaggctggatcttcggcaccacactggacagcaagacacagtccctgctgatcgtgaacaatgccaccaacgtggtcatcaaggtgtgcgagtttcagttctgtaatgatccttttctgggcgtgtactatcacaagaacaataagtcttggatggagagcgagttccgcgtgtattcctctgccaacaattgtacattcgagtacgtgtcccagccatttctgatggacctggagggcaagcagggcaacttcaagaacctgcgggagttcgtgttcaagaatatcgatggctatttcaagatctactctaagcacacccctatcaacctggtgcgcgacctgccacagggctttagcgccctggagcctctggtggatctgccaatcggcatcaacatcaccaggttccagacactgctggccctgcaccgcagctacctgacacctggcgacagctcctctggatggaccgccggggccgccgcctactatgtgggctatctgcagccacggacctttctgctgaagtacaacgagaatggcaccatcacagacgcagtggattgcgccctggaccccctgagcgagaccaagtgtacactgaagtccttcaccgtggagaagggcatctatcagacatccaattttcgggtgcagcccaccgagtctatcgtgagattcccaaatatcacaaacctgtgccccttcggcgaggtgtttaacgccaccagattcgccagcgtgtacgcctggaatcggaagagaatctctaactgcgtggccgactatagcgtgctgtacaactctgccagcttttccaccttcaagtgctatggcgtgtcccccacaaagctgaatgacctgtgcttcaccaacgtgtacgccgattcttttgtgatcaggggcgacgaggtgagacagatcgcaccaggccagacaggcaagatcgccgactacaattataagctgcccgacgatttcaccggctgcgtgatcgcctggaactctaacaatctggatagcaaagtgggcggcaactacaattatctgtacaggctgttccgcaagagcaatctgaagccttttgagcgggacatctctacagagatctaccaggccggcagcaccccatgcaatggcgtggagggcttcaactgttattttcccctgcagtcctacggctttcagcctaccaacggcgtgggctatcagccatacagagtggtggtgctgagcttcgagctgctgcacgcaccagcaacagtgtgcggacctaagaagtccaccaatctggtgaagaacaagtgcgtgaacttcaacttcaacggcctgaccggcacaggcgtgctgaccgagtccaataagaagtttctgcccttccagcagtttggccgggacatcgccgataccacagacgccgtgagagacccccagaccctggagatcctggacatcacaccttgctccttcggcggcgtgtctgtgatcacacctggcaccaatacaagcaaccaggtggccgtgctgtatcaggacgtgaattgtaccgaggtgccagtggccatccacgccgatcagctcacccccacatggcgggtgtactccaccggctctaacgtgttccagacaagagccggctgcctgatcggagccgagcatgtgaacaattcctatgagtgcgacatccccatcggagccggcatctgtgcctcttaccagacccagacaaacagccctggctccgccagctccgtggcctctcagagcatcatcgcctataccatgagcctgggggccgagaacagcgtggcctactctaacaatagcatcgccatccccaccaactttacaatctctgtgaccacagagatcctgcctgtgagcatgaccaagacatccgtggactgcacaatgtatatctgtggcgattccaccgagtgctctaacctgctgctgcagtacggcagcttctgtacccagctcaaccgggccctgacaggaatcgcagtggagcaggacaagaacacacaggaggtgtttgcccaggtgaagcagatctacaagaccccacccatcaaggacttcggcggctttaatttctcccagatcctgccagatccctccaagccatctaagcggagcttcatcgaggacctgctgtttaacaaggtgaccctggccgatgccggctttatcaagcagtatggcgattgcctgggcgacatcgccgccagagacctgatctgtgcccagaagttcaatggcctgaccgtgctgcctccactgctgacagatgagatgatcgcacagtacacaagcgccctgctggccggcaccatcacatccggatggaccttcggggccggggccgccctgcagatccccttcgccatgcagatggcctataggtttaacggcatcggcgtgacccagaatgtgctgtacgagaaccagaagctgatcgccaatcagttcaactccgccatcggcaagatccaggacagcctgtctagcacagcctccgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgacaggctgatcacaggccgcctgcagtctctgcagacctatgtgacacagcagctcatcagggccgccgagatcagagccagcgccaatctggccgccaccaagatgtccgagtgcgtgctgggccagtctaagcgcgtggacttctgtggcaagggctatcacctgatgagctttccacagtccgccccccacggagtggtgttcctgcatgtgacctacgtgcctgcccaggagaagaactttaccacagccccagccatctgccacgatggcaaggcacacttccccagggagggcgtgttcgtgagcaacggcacccactggttcgtgacacagcgcaacttctacgagcctcagatcatcaccacagacaatacattcgtgtctggcaactgtgacgtggtcatcggcatcgtgaacaataccgtgtatgatcccctgcagcctgagctggacagcttcaaggaggagctggataagtactttaagaatcacacctcccccgacgtggatctgggcgacatctctggcatcaatgccagcgtggtgaacatccagaaggagatcgacaggctgaacgaggtggccaagaatctgaacgagagcctgatcgatctgcaggagctgggcaagtatgagcagtacatcaagttctttttcatcatcggcctgatcatcggcctgttcctggtgctgcgcgtgggcatccacctgtgcatcaagctgaagcacaccaagaagaggcagatctacacagacatcgagatgaaccgcctgggcaagtga

SEQ ID NO: 25 - protein, 1255 aa, Artificial Sequence
  Description of Artificial Sequence Synthetic polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 26 - DNA, 3819 bp, Artificial Sequence
  Description of Artificial Sequence Synthetic polynucleotide
ctcgaggtttaaacgaattccgccaccatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatttttctttatcatagggttaatcattggactattcttggttctccgagttggtatccatctttgcattaaattaaagcacaccaagaaaagacagatttatacagacatagagatgaaccgacttggaaagtaagaattccacgtgggatcc

SEQ ID NO: 27 - DNA, 3819 bp, Artificial Sequence
  Description of Artificial Sequence Synthetic polynucleotide
ctcgaggtttaaacgaattccgccaccatgttcgtgttcctggtgctgctgcccctggtgtccagccagtgcgtgaatctgaccacccggacccaactgcctcccgcctacacaaactctttcaccagaggggtttattaccccgataaggtgttcagaagctcagtgcttcattctacccaggacctgtttctgccttttttcagcaacgtcacatggttccacgccatccacgtcagcggaaccaacggcacgaagcggttcgacaatcctgtgctgccttttaacgacggcgtctactttgccagcacggaaaagagcaacattatccggggatggatcttcggcaccaccctggactctaaaacccagagcctgttgatcgtgaacaacgcaaccaatgtggtgatcaaggtctgcgagttccaattttgcaacgatcctttcctgggcgtgtactaccacaagaacaacaagtcttggatggaatctgagttccgcgtctacagcagcgcaaacaactgcacatttgagtacgtgtctcagccttttctgatggacctggaaggaaagcagggaaatttcaagaacctgcgggagttcgtgttcaagaacatcgacggctacttcaagatctacagcaagcacacccccatcaacctcgtgagagacctgccccagggcttcagcgccctggaacccctggtggaccttcccataggaatcaacatcacacggttccagacactgctggccctgcatagaagctacctgacccctggagattctagcagcggctggaccgccggcgctgccgcttactacgtcggatacctgcagcctagaaccttcctgttgaagtacaacgagaacggcaccatcacagatgccgtggactgcgccctggaccccctgagcgagacaaagtgcaccctgaagagcttcaccgtggagaagggcatctaccagacaagcaacttcagagtgcagcctaccgagtcaatcgtgagattcccaaacatcaccaacctttgtcctttcggcgaggtatttaacgccacccggttcgccagcgtgtacgcctggaataggaagcggatcagcaactgcgtggccgattacagcgtgctctataacagcgccagttttagcactttcaagtgctacggagtctctcctacaaagctgaacgacctgtgcttcaccaacgtgtatgccgacagcttcgtcatccggggcgacgaggtgcgacagatcgctcctggccagaccggcaagatagccgactacaactacaagctgcctgacgacttcacaggctgcgtgatcgcttggaacagcaacaatctggatagcaaagtgggcggcaactataactacctgtacagactgttccggaagtccaatctcaagccgtttgagagagacatcagcaccgaaatctaccaggctggatctacaccctgcaacggcgtcgaaggcttcaattgttacttccctctgcaatcttacggcttccagcccaccaacggcgtgggctaccagccctacagagtggttgtgctgagcttcgagctgctgcacgccccagctacagtgtgcggccctaagaaatctacaaacctggtcaagaacaagtgtgtgaacttcaacttcaatggcctgacgggcaccggcgtgctgacagagagcaacaagaagttcctgcctttccagcaatttggcagagacatcgccgacaccaccgacgccgtgcgcgaccctcagaccctggaaattctggacatcaccccatgttctttcggcggcgtgtccgtcattacgccaggcaccaataccagcaaccaggtggccgtgctttatcaggatgtgaattgtaccgaagttcctgttgcaatccacgccgaccaactgacccccacatggagagtgtactctaccggcagcaacgtgttccaaacgagagccggatgcctgattggagctgagcatgtgaacaacagctacgagtgcgatattccaatcggagccggcatctgcgcctcctaccaaacacaaaccaactcccctcgtagagcgagaagcgtggcctctcagagcatcatcgcctacaccatgagcctgggtgccgaaaactccgtggcttactccaacaacagcatcgccatccctacaaatttcaccatcagcgtgacaaccgagatcctgcctgtgtccatgaccaagaccagcgtggactgcacgatgtacatctgcggagatagcaccgagtgcagcaatctgctactgcagtatggcagcttctgcacccaactgaacagagcactgaccggcattgctgtggaacaggacaagaatacccaggaggtgttcgcccaagtgaagcagatttacaagacaccccctatcaaggacttcggaggcttcaacttcagccagatcctgcctgaccctagcaagccaagcaaaagatcctttatcgaagatctgctgtttaacaaggtgacactggccgatgccggctttatcaagcagtacggcgactgcctgggagacatcgccgccagagacctgatctgtgctcagaaatttaacgggctgaccgtgctgccacctctgctgacagatgagatgatcgctcagtacaccagcgccctgctggccggcacaattacctccggctggaccttcggagccggagccgccctgcagatccccttcgccatgcagatggcctaccggttcaatggcatcggcgtcacccaaaacgtgctctatgagaaccagaagctgatcgcaaaccagttcaactccgccatcggtaagatccaggacagtctgagcagcacggcgtctgccctgggcaagctccaggacgtggtgaaccagaacgcccaggcccttaacaccctggtgaaacaactgagcagcaacttcggtgccatttccagcgttctcaatgacatcctgagcagactggataaggtggaagccgaggtgcagatcgaccggctgatcaccggacggctgcagagcctgcagacgtacgtgacccagcaattaatcagagctgccgagatcagagccagcgccaatctggctgccaccaaaatgagcgaatgtgtgctgggccagtcaaagagagtggatttttgtggcaaaggctaccacctgatgtccttccctcagtctgcccctcacggcgtggtgttcctccatgtgacctatgtgcctgctcaggagaagaactttaccacagcccctgctatctgccacgacggaaaggcccacttccccagagagggcgtgtttgtgtccaatggcacacactggttcgtgacccaaagaaacttctacgagccccagatcatcaccacagacaacaccttcgtgagcggcaactgcgacgtggtgatcggcatcgtgaacaacacagtgtacgaccccctgcaacctgagctggacagcttcaaagaggaactggacaaatacttcaagaatcacaccagccctgatgtggatctgggcgacatcagcggcatcaacgccagcgtcgtgaacatccagaaggaaatcgacagactgaacgaagtggccaagaacctgaacgagagcctcatcgatctgcaggagctgggcaagtacgagcagtacatcaaatggcctttcttcttcatcatcggcctgattatcggcctgttcctcgtgctgagagtgggcatccacctgtgcatcaagcttaagcacacaaaaaagcggcagatttacaccgacatcgagatgaaccggctgggcaaatgagaattccacgtgggatcc

SEQ ID NO: 28 - protein, 1252 aa, Artificial Sequence
  Description of Artificial Sequence Synthetic polypeptide
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGSASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKFFFIIGLIIGLFLVLRVGIHLCIKLKHTKKRQIYTDIEMNRLGK

SEQ ID NO: 29 - DNA, 3759 bp, Artificial Sequence
  Description of Artificial Sequence Synthetic polynucleotide
atgtttgtttttcttgttttattgccactagtctctagtcagtgtgttaattttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgctaaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtggtctccctcagggtttttcggctttagaaccattggtagatttgccaataggtattaacatcactaggtttcaaactttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctggtagtgcaagtagtgtagctagtcaatccatcattgcctacactatgtcacttggtgtagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgaccctcctgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatttttctttatcatagggttaatcattggactattcttggttctccgagttggtatccatctttgcattaaattaaagcacaccaagaaaagacagatttatacagacatagagatgaaccgacttggaaagtaa

SEQ ID NO: 30 - DNA, 3759 bp, Artificial Sequence
  Description of Artificial Sequence Synthetic polynucleotide
atgttcgtgttcctggtgctgctgcctctggtgagctcccagtgcgtgaacttcaccacaaggacccagctcccccctgcctacaccaattcctttacaaggggcgtgtactatccagacaaggtgttccgctctagcgtgctgcactctacacaggatctgttcctgcccttctttagcaacgtgacctggtttcacgccatccatgtgagcggcaccaatggcacaaagcggtttgccaatcctgtgctgccattcaacgacggcgtgtattttgcctccaccgagaagtctaacatcatcagaggctggatcttcggcaccacactggatagcaagacacagtccctgctgatcgtgaacaatgccaccaacgtggtcatcaaggtgtgcgagtttcagttctgtaatgacccttttctgggcgtgtactatcacaagaacaataagtcttggatggagagcgagttccgcgtgtactcctctgccaacaattgtacattcgagtacgtgagccagccatttctgatggacctggagggcaagcagggcaacttcaagaacctgcgggagttcgtgttcaagaatatcgatggctatttcaagatctactctaagcacacccctatcaacctggtgcgcggactgccacagggctttagcgccctggagcctctggtggacctgccaatcggcatcaacatcaccaggttccagacactgcaccgcagctacctgacacctggcgatagctcctctggatggaccgccggggccgccgcctactatgtgggctacctgcagccacggacctttctgctgaagtataacgagaatggcaccatcacagacgcagtggattgcgccctggaccccctgagcgagaccaagtgtacactgaagtccttcaccgtggagaagggcatctaccagacatccaattttcgggtgcagcccaccgagtctatcgtgagattcccaaatatcacaaacctgtgccccttcggcgaggtgtttaacgccaccagattcgccagcgtgtatgcctggaatcggaagagaatctctaactgcgtggccgactatagcgtgctgtacaactctgccagcttttccaccttcaagtgctatggcgtgtcccccacaaagctgaatgacctgtgcttcaccaacgtgtatgccgattcttttgtgatcaggggcgacgaggtgagacagatcgcaccaggccagacaggcaatatcgccgactacaactataagctgcccgacgatttcaccggctgcgtgatcgcctggaacagcaacaatctggatagcaaagtgggcggcaactacaattatctgtacaggctgttccgcaagagcaacctgaagccttttgagcgggacatctctacagagatctaccaggccggcagcaccccatgcaatggcgtgaagggcttcaactgttattttcccctgcagtcctacggctttcagcctacctatggcgtgggctatcagccatacagagtggtggtgctgagcttcgagctgctgcacgcaccagcaacagtgtgcggacctaagaagtccaccaatctggtgaagaacaagtgcgtgaacttcaacttcaacggcctgaccggcacaggcgtgctgaccgagtccaataagaagtttctgcccttccagcagtttggccgggacatcgccgataccacagacgccgtgagagacccccagaccctggagatcctggacatcacaccttgctcctttggcggcgtgtctgtgatcacacctggcaccaatacaagcaaccaggtggccgtgctgtaccagggcgtgaattgtaccgaggtgccagtggccatccacgcagaccagctcacccccacatggcgggtgtattccaccggctctaacgtgttccagacaagagccggctgcctgatcggagccgagcatgtgaacaattcctacgagtgcgacatccccatcggagccggcatctgtgcctcttatcagacccagacaaacagccctggctccgccagctccgtggcctctcagagcatcatcgcctacaccatgtccctgggcgtggagaacagcgtggcctattctaacaatagcatcgccatccccaccaactttacaatctctgtgaccacagagatcctgcctgtgagcatgaccaagacatccgtggactgcacaatgtacatctgtggcgattccaccgagtgctctaacctgctgctgcagtatggcagcttctgtacccagctcaaccgggccctgacaggaatcgcagtggagcaggacaagaacacacaggaggtgtttgcccaggtgaagcagatctacaagaccccacccatcaaggacttcggcggctttaatttctcccagatcctgccagatccctccaagccatctaagcggagcttcatcgaggacctgctgtttaacaaggtgaccctggccgatgccggctttatcaagcagtacggcgattgcctgggcgacatcgcagccagagacctgatctgtgcccagaagttcaatggcctgaccgtgctgcctccactgctgacagatgagatgatcgcccagtatacaagcgccctgctggccggcaccatcacatccggatggaccttcggggccggggccgccctgcagatccccttcgccatgcagatggcctacaggtttaacggcatcggcgtgacccagaatgtgctgtatgagaaccagaagctgatcgccaatcagttcaactccgccatcggcaagatccaggacagcctgtctagcacagcctccgccctgggcaagctgcaggatgtggtgaatcagaacgcccaggccctgaataccctggtgaagcagctcagcagcaacttcggggccatcagcagcgtgctgaacgacatcctgagccggctggacccccctgaggcagaggtgcagatcgacaggctgatcacaggccgcctgcagtctctgcagacctacgtgacacagcagctcatcagggccgccgagatcagagccagcgccaatctggccgccaccaagatgtccgagtgcgtgctgggccagtctaagcgcgtggacttctgtggcaagggctaccacctgatgagctttccacagtccgccccccacggagtggtgttcctgcatgtgacctatgtgcctgcccaggagaagaactttaccacagccccagccatctgccacgatggcaaggcacacttccccagggagggcgtgttcgtgagcaacggcacccactggttcgtgacacagcgcaacttctacgagcctcagatcatcaccacagacaatacattcgtgtctggcaactgtgacgtggtcatcggcatcgtgaacaataccgtgtacgatcccctgcagcctgagctggacagcttcaaggaggagctggataagtacttcaagaatcacacctcccccgacgtggatctgggcgacatctctggcatcaatgccagcgtggtgaacatccagaaggagatcgacaggctgaacgaggtggccaagaatctgaacgagagcctgatcgatctgcaggagctgggcaagtatgagcagtacatcaagttctttttcatcatcggcctgatcatcggcctgttcctggtgctgcgcgtgggcatccacctgtgcatcaagctgaagcacaccaagaagaggcagatctacacagacatcgagatgaaccgcctgggcaagtga

SEQ ID NO: 31 - DNA, 26 bp, Artificial Sequence
  Description of Artificial Sequence Synthetic primer
acaggtacgttaatagttaatagcgt

SEQ ID NO: 32 - DNA, 22 bp, Artificial Sequence
  Description of Artificial Sequence Synthetic primer
atattgcagcagtacgcacaca

SEQ ID NO: 33 - DNA, 26 bp, Artificial Sequence
  Description of Artificial Sequence Synthetic probe
acactagccatccttactgcgcttcg

SEQ ID NO: 34 - DNA, 22 bp, Artificial Sequence
  Description of Artificial Sequence Synthetic primer
ccacggagaaagacctcatctg

SEQ ID NO: 35 - DNA, 24 bp, Artificial Sequence
  Description of Artificial Sequence Synthetic primer
gttgccaaaccttatcagaaatga

SEQ ID NO: 36 - DNA, 18 bp, Artificial Sequence
  Description of Artificial Sequence Synthetic primer
aaatggcgggttctgtgc

SEQ ID NO: 37 - DNA, 19 bp, Artificial Sequence
  Description of Artificial Sequence Synthetic primer
ggagtggctgagccatcgt

SEQ ID NO: 38 - DNA, 20 bp, Artificial Sequence
  Description of Artificial Sequence Synthetic primer
ggccatccagaggagcatag

SEQ ID NO: 39 - DNA, 19 bp, Artificial Sequence
  Description of Artificial Sequence Synthetic primer
actgccgcatcctcttcct

SEQ ID NO: 40 - DNA, 24 bp, Artificial Sequence
  Description of Artificial Sequence Synthetic primer
gggtcacctcatgttggaaataaa

SEQ ID NO: 41 - DNA, 19 bp, Artificial Sequence
  Description of Artificial Sequence Synthetic primer
ttctggcccgtggttctct

SEQ ID NO: 42 - DNA, 25 bp, Artificial Sequence
  Description of Artificial Sequence Synthetic primer
aatatcctctgggtcttgtagatgg

SEQ ID NO: 43 - DNA, 24 bp, Artificial Sequence
  Description of Artificial Sequence Synthetic primer
agctggttgtctttgagagacatg

SEQ ID NO: 44 - DNA, 21 bp, Artificial Sequence
  Description of Artificial Sequence Synthetic primer
tttctccatgctgctgttgaa

SEQ ID NO: 45 - DNA, 20 bp, Artificial Sequence
  Description of Artificial Sequence Synthetic primer
tcgttgccaatggtgatgac

SEQ ID NO: 46 - protein, 4 aa, Severe acute respiratory syndrome coronavirus 2
RRAR

SEQ ID NO: 47 - protein, 4 aa, Artificial Sequence
  Description of Artificial Sequence Synthetic peptide
GSAS

(19) United States
(12) Patent Application Publication
(10) Pub. No.: US 2022/0233682 A1
(43) Pub. Date: Jul. 28, 2022

(54) VACCINE COMPOSITIONS FOR THE TREATMENT OF CORONAVIRUS

(71) Applicant: Variation Biotechnologies Inc., Ottawa (CA)

(72) Inventors: David Evander Anderson, Newton, MA (US); Anne-Catherine Fluckiger, Saint Genis Les Ollières (FR)

(21) Appl. No.: 17/573,806

(22) Filed: Jan. 12, 2022

Related U.S. Application Data
(63) Continuation-in-part of application No. 17/218,148, filed on Mar. 30, 2021, now Pat. No. 11,253,587.
(60) Provisional application No. 63/070,150, filed on Aug. 25, 2020.
(60) Provisional application No. 63/002,237, filed on Mar. 30, 2020.

(51) Int. Cl. A61K 39/215; A61K 39/21; A61K 39/205; A61K 39/39; A61P 31/14

(52) U.S. Cl. CPC A61K 39/215; A61K 39/21; A61K 2039/5258; A61K 39/39; A61P 31/14; A61K 39/205

(57) ABSTRACT

The present disclosure provides compositions and methods useful for preventing and/or treating coronavirus infection. As described herein, the compositions and methods are based on development of immunogenic compositions that include virus-like particles (VLPs) which comprise one or more Moloney Murine leukemia virus (MMLV) core proteins and include a spike protein from the Beta variant of SARS-Cov-2.

### CROSS REFERENCE TO RELATED APPLICATIONS

This application is a continuation-in-part of U.S. application Ser. No. 17/218,148, filed Mar. 30, 2021 which claims the benefit of U.S. Provisional Application No. 63/002,237, filed Mar. 30, 2020, and of U.S. Provisional Application No. 63/070,150, filed Aug. 25, 2020, the contents of each of which are hereby incorporated herein in their entireties.

### SEQUENCE LISTING

The instant application contains a Sequence Listing which has been submitted electronically in ASCII format and is hereby incorporated by reference in its entirety. Said ASCII copy, created on Mar. 31, 2022, is named 2007801-0148_SL.txt and is 205,747 bytes in size.

### FIELD OF THE INVENTION

This invention is in the field of vaccines, in particular virus like particle vaccines for coronavirus.

### BACKGROUND

Coronaviruses are spherical, enveloped viruses, ranging from 160-180 nm in diameter and containing a positive-stranded RNA genome. With their genome of approximately 30,000 bases, they are considered the largest of the known RNA viruses. Like influenza viruses they have the ability to genetically recombine with other members of the coronavirus family. Coronaviruses fall into four major genera. Coronaviruses are believed to be the causative agents of several severe diseases in many animals, for example, infectious bronchitis virus, feline infectious peritonitis virus and transmissible gastroenteritis virus. Coronaviruses also cause a range of illnesses in humans from the common cold to severe respiratory infections. Four human coronaviruses, HCoV-0C43, HCoV-HKU1 (betacoronaviruses), and HCoV-NL63, HCoV-229E (alphacoronaviruses), contribute to 15%-30% of common colds (Fung et al (2019) Annu. Rev. Microbiol. 73:2-529-557). In recent years, beta-coronaviruses have been responsible for three significant outbreaks of disease in humans.

In the early 2000s, a beta coronavirus known as SARS-CoV caused an outbreak of respiratory disease referred to as severe acute respiratory syndrome (SARS). The main symptoms included fever, dry cough, headache, shortness of breath and difficulty of breathing. Many of those infected developed viral pneumonia resulting in infection of the lower respiratory tract. SARS is highly contagious, and is spread by droplets caused by coughing or sneezing or through other methods such as fecal contamination. SARS was fatal in around 9.14% of all cases. The global outbreak of SARS was contained in July 2003 and there have been no reported cases since 2004 (Peeri et al Int. J. Epi, Feb. 10, 2020).

In 2012, another novel coronavirus emerged in Saudi Arabia which is now known as Middle East Respiratory Syndrome coronavirus (MERS-CoV). MERS-CoV is also beta coronavirus. Subsequent cases of MERS-CoV infection were reported and the outbreak spread to 27 countries in the Middle East, Europe, Asia and North America. Infection with MERS-CoV presented as a severe acute respiratory illness with symptoms of fever, cough, and shortness of breath. About 34% of reported cases of MERS-CoV infection resulted in death. Only a small number of reported cases involved subjects with mild respiratory illness.

In late 2019, a respiratory infection appeared in Wuhan, China which was quickly identified as caused by a novel coronavirus strain called SARS-CoV-2. The infection, known as COVID-19 is highly infectious and causes severe pneumonia, particularly in elderly patients. Mortality rates vary significantly by country, with estimates ranging from 13.7% in Italy to 1.9% in Japan. As of March 2021, the fatality rate in the United States was approximately 1.8% (Johns Hopkins Coronavirus Research Centre, Update as of Mar. 30, 2021). COVID-19 quickly spread throughout the world resulting in a significant threat to human health and a massive slowdown in economic activity. As of Feb. 1, 2021, more than 100 million people had contracted COVID-19, and over 2 million had died.

In late 2020, several vaccines against COVID-19 were approved for emergency use. These vaccines target a protein on the surface of SARS-CoV-2 known as the spike protein and utilized novel platforms, sometimes for the first time for human use. These vaccines were shown to be highly effective in clinical trials, but distribution has been slow in many parts of the world due to manufacturing challenges and, in some cases, the requirement for storage at ultra-low temperatures. Furthermore, while several new vaccines have proven to be safe, some have been associated with rare but deadly side effects that have restricted their use in certain countries.

During the second half of 2020, variants of SARS-CoV-2 emerged which cause COVID-19 disease, and those variants which had an impact on transmissibility, severity of disease and/or immunity were designated Variants of Concern (VoCs). Three VoCs rapidly became dominant during this period in the countries where they were first detected, B.1.1.7 first identified in the UK (also known as the Alpha variant), 501Y.V2 or B.1351 first identified in South Africa (also known as the Beta variant), and P.1 first identified in Brazil (also known as the Gamma variant). More recently, new VoCs emerged in India (B.1617 also known as the Delta variant) and in South Africa (B.1.1.529 also known as the Omicron variant). These VoCs have proven to be highly infectious due to increased binding affinity of the viral receptor-binding domain (RBD) to the receptor known as angiotensin-converting enzyme 2 (ACE2). Each of the VoCs are characterized by a number of shared mutations expressed on the spike protein, primarily located in the RBD and N-terminal domain (NTD), that serve to increase transmission and/or enhance escape from neutralizing antibodies acquired by vaccination or prior natural SARS-CoV-2 infection. The rapid spread of the new VoCs, and the possible emergence of new variants has raised significant concerns regarding reinfection and the effectiveness of the recently approved vaccines, all of which were developed against the original strain of SARS-CoV-2.

As a result, there is an urgent need to develop new vaccines which induce strong immunity against SARS-CoV-2 while being safe and easy to store and distribute. Furthermore, there is an urgent need to ensure that vaccines against SARS-CoV-2 provide broad immunity so as to protect patients against mutated forms of the virus including VoCs.

Accordingly, a need exists for a vaccine against human coronaviruses which provides broad immunity against coronavirus antigens.

### SUMMARY

The present disclosure provides methods and compositions useful for prophylaxis of infection cause by human coronaviruses. More particularly, the present disclosure provides methods for production of, and compositions comprising, virus like particles (VLPs) expressing antigens from human coronaviruses which are useful for prevention, treatment, and/or diagnosis of infections caused by coronaviruses.

The present disclosure provides virus-like particles which comprise one or more Moloney Murine leukemia virus (MMLV) core proteins and are surrounded by a lipid bilayer membrane. The VLPs include one or more envelope polypeptides from human coronaviruses (e.g., one or more coronavirus polypeptide epitopes) that play a role in induction of virus-neutralizing antibodies.

In some embodiments, the present disclosure provides VLPs having an envelope that comprises a wild type human coronavirus envelope glycoprotein. In some embodiments, the polypeptide is from SARS-CoV. In some embodiments, the polypeptide is from MERS-CoV. In some embodiments, the polypeptide is from SARS-CoV-2. The polypeptides from SARS-CoV-2 can be from the ancestral strain, first identified in Wuhan China, or from a variant of the ancestral strain. In some embodiments, the VLPs include polypeptides from more than one of SARS-CoV, MERS-CoV and SARS-CoV-2. In some embodiments, the VLPs include polypeptides from all three of SARS-CoV, MERS-CoV and SARS-CoV-2.

In some embodiments, the present disclosure provides VLPs having an envelope that comprises a modified human coronavirus envelope glycoprotein. In an embodiment, the present disclosure encompasses production of VLPs having envelopes that include a coronavirus polypeptide in a premature conformation. In a specific embodiment, the modified envelope glycoprotein lacks a furin cleavage site. In some embodiments, the polypeptide lacking a furin cleavage site is from SARS-CoV. In some embodiments, the polypeptide lacking a furin cleavage site is from MERS-CoV. In some embodiments, the polypeptide lacking a furin cleavage site is from SARS-CoV-2. In some embodiments, the VLPs include polypeptides from more than one of SARS-CoV, MERS-CoV and SARS-CoV-2, wherein the polypeptides lack a furin cleavage site. In some embodiments, the VLPs include polypeptides from all three of SARS-CoV, MERS-CoV and SARS-CoV-2, wherein the polypeptides lack a furin cleavage site.

In another embodiment, the present disclosure encompasses production of VLPs having envelopes that include a coronavirus polypeptide having a modified amino acid sequence. In a specific embodiment, the modified envelope glycoprotein has a lysine and valine residue substituted for proline residues. In some embodiments, the polypeptide having a proline substitution is from SARS-CoV. In some embodiments, the polypeptide having a proline substitution is from MERS-CoV. In some embodiments, the polypeptide having a proline substitution is from SARS-CoV-2. In some embodiments, the VLPs include polypeptides from more than one of SARS-CoV, MERS-CoV and SARS-CoV-2, wherein the polypeptides have a proline substitution. In some embodiments, the VLPs include polypeptides from all three of SARS-CoV, MERS-CoV and SARS-CoV-2, wherein the polypeptides have a proline substitution.

In another embodiment, the present disclosure encompasses production of VLPs having envelopes that include a coronavirus polypeptide having a modified amino acid sequence and a premature conformation. In a specific embodiment, the modified envelope glycoprotein has a lysine and valine residue substituted for proline residues and lack a furin cleavage site. In some embodiments, the polypeptide having a proline substitution and lacking a furin cleavage site is from SARS-CoV. In some embodiments, the polypeptide having a proline substitution and lacking a furin cleavage site is from MERS-CoV. In some embodiments, the polypeptide having a proline substitution and lacking a furin cleavage site is from SARS-CoV-2. In some embodiments, the VLPs include polypeptides from more than one of SARS-CoV, MERS-CoV and SARS-CoV-2, wherein the polypeptides have a proline substitution and lack a furin cleavage site. In some embodiments, the VLPs include polypeptides from all three of SARS-CoV, MERS-CoV and SARS-CoV-2, wherein the polypeptides have a proline substitution and lack a furin cleavage site.

In a further embodiment, the modified envelope glycoprotein has been modified such that the transmembrane domain is replaced with the transmembrane domain of another virus. In a particularly preferred embodiment, the VLP has a modified envelope glycoprotein comprising an isolated coronavirus S protein, the transmembrane domain and cytoplasmic tail of which protein have been replaced with the transmembrane domain and cytoplasmic tail from vesicular stomatitis virus (VSV). In some embodiments, the polypeptide having a transmembrane domain and cytoplasmic tail from VSV is from SARS-CoV. In some embodiments, the polypeptide having a transmembrane domain and cytoplasmic tail from VSV is from MERS-CoV. In some embodiments, the polypeptide having a transmembrane domain and cytoplasmic tail from VSV is from SARS-CoV-2. In some embodiments, the VLPs include polypeptides from more than one of SARS-CoV, MERS-CoV and SARS-CoV-2, wherein the polypeptides have a transmembrane domain and cytoplasmic tail from VSV. In some embodiments, the VLPs include polypeptides from all three of SARS-CoV, MERS-CoV and SARS-CoV-2, wherein the polypeptides have a transmembrane domain and cytoplasmic tail from VSV. In some embodiments, the VLPS include one or more polypeptides from SARS-CoV, MERS-CoV and SARS-CoV-2, one or more of which have been modified as described herein and which have a transmembrane domain and cytoplasmic tail from VSV.

In a preferred embodiment, the present disclosure encompasses production of a VLP having an envelope that includes a SARS-CoV-2 spike polypeptide having a modified amino acid sequence and a premature conformation. The modified envelope glycoprotein has a lysine and valine residue substituted for proline residues and it lacks a furin cleavage site. Furthermore, the modified spike glycoprotein has been further modified such that the transmembrane domain and cytoplasmic tail have been replaced with the transmembrane domain and cytoplasmic tail from vesicular stomatitis virus (VSV).

The present disclosure further provides bivalent and trivalent VLPs comprising one or more modified human coronavirus envelope proteins and one or more wild type human coronavirus proteins.

The present disclosure further provides VLPs having an envelope that includes a SARS-CoV-2 spike polypeptide, wild type or modified, from a variant of the ancestral SARS-CoV-2 strain which comprises a heterologous booster vaccine. In a preferred embodiment, the VLP includes a modified SARS-CoV-2 spike polypeptide from the Beta variant. In a particularly preferred embodiment, the SARS-CoV-2 spike polypeptide from the Beta variant has a lysine and valine residue substituted for proline residues, it lacks a furin cleavage site and has been further modified such that the transmembrane domain and cytoplasmic tail have been replaced with the transmembrane domain and cytoplasmic tail from vesicular stomatitis virus (VSV).

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 is a diagram illustrating the structure of the SARS-CoV-2 envelope.

FIG. 2 the S1/S2 domains from SARS-CoV, SARS CoV-2 and MERS-CoV.

FIG. 3 is a diagram illustrating exemplary alternative COVID-S constructs.

FIG. 3 discloses “RRAR” as SEQ ID NO:46 and “GSAS” as SEQ ID NO:47.

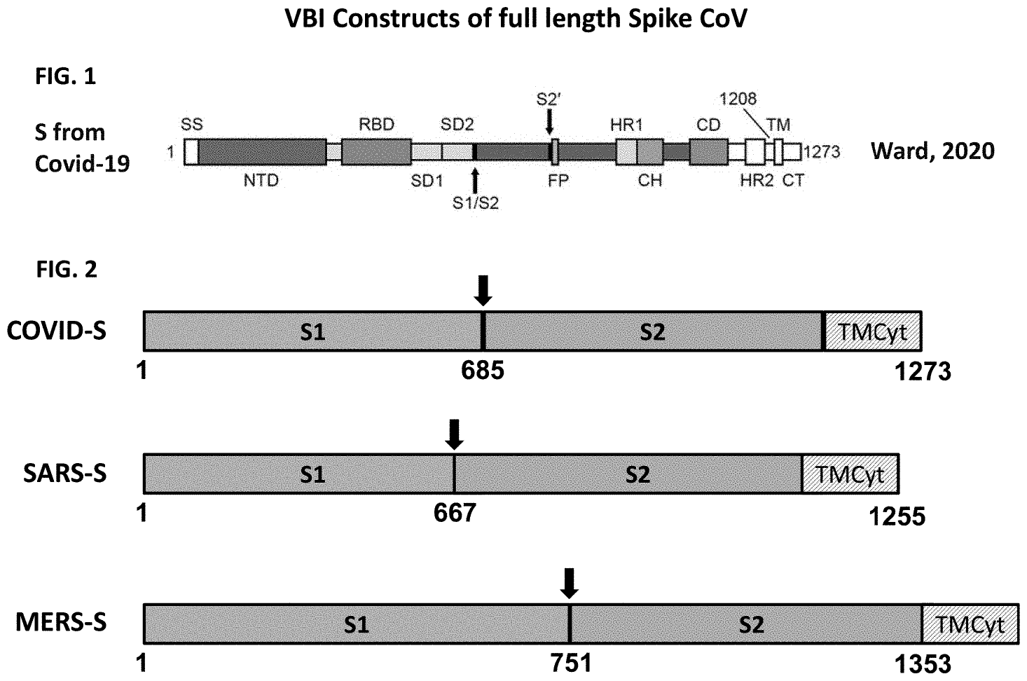

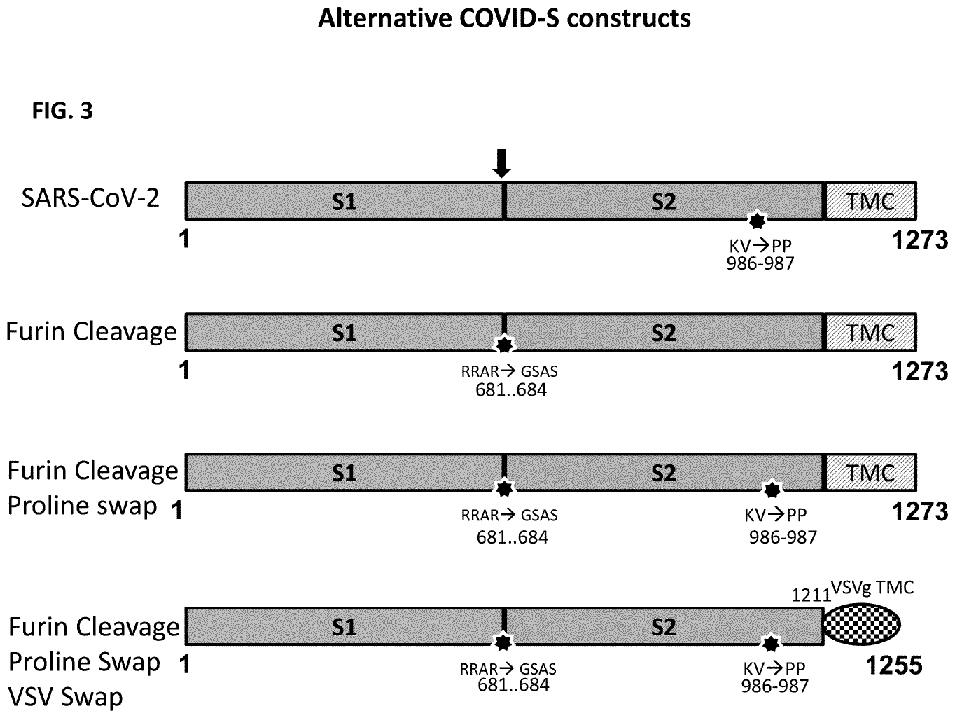

Listing of Sequences

The following is a list of sequences referred to herein:
SEQ ID NO: 1 is an MMLV-Gag Amino Acid Sequence
MGQTVTTPLSLTLGHWKDVERIAHNQSVDVKKRRWVTFCSAEWPTFNVGWPRDGTFNR
DLITQVKIKVFSPGPHGHPDQVPYIVTWEALAFDPPPWVKPFVHPKPPPPLPPSAPSLPLEP
PRSTPPRSSLYPALTPSLGAKPKPQVLSDSGGPLIDLLTEDPPPYRDPRPPPSDRDGNGGEA
TPAGEAPDPSPMASRLRGRREPPVADSTTSQAFPLRAGGNGQLQYWPFSSSDLYNWKNN
NPSFSEDPGKLTALIESVLITHQPTWDDCQQLLGTLLTGEEKQRVLLEARKAVRGDDGRP
TQLPNEVDAAFPLERPDWDYTTQAGRNHLVHYRQLLLAGLQNAGRSPTNLAKVKGITQ
GPNESPSAFLERLKEAYRRYTPYDPEDPGQETNVSMSFIWQSAPDIGRKLERLEDLKNKTL
GDLVREAEKIFNKRETPEEREERIRRETEEKEERRRTEDEQKEKERDRRRHREMSKLLAT
VVSGQKQDRQGGERRRSQLDRDQCAYCKEKGHWAKDCPKKPRGPRGPRPQTSLLTLDD
SEQ ID NO: 2 is MMLV-Gag Nucleotide Sequence
ATGGGCCAGACTGTTACCACTCCCTTAAGTTTGACCTTAGGTCACTGGAAAGATGTCG
AGCGGATCGCTCACAACCAGTCGGTAGATGTCAAGAAGAGACGTTGGGTTACCTTCT
GCTCTGCAGAATGGCCAACCTTTAACGTCGGATGGCCGCGAGACGGCACCTTTAACC
GAGACCTCATCACCCAGGTTAAGATCAAGGTCTTTTCACCTGGCCCGCATGGACACCC
AGACCAGGTCCCCTACATCGTGACCTGGGAAGCCTTGGCTTTTGACCCCCCTCCCTGG
GTCAAGCCCTTTGTACACCCTAAGCCTCCGCCTCCTCTTCCTCCATCCGCCCCGTCTCT
CCCCCTTGAACCTCCTCGTTCGACCCCGCCTCGATCCTCCCTTTATCCAGCCCTCACTC
CTTCTCTAGGCGCCAAACCTAAACCTCAAGTTCTTTCTGACAGTGGGGGGCCGCTCAT
CGACCTACTTACAGAAGACCCCCCGCCTTATAGGGACCCAAGACCACCCCCTTCCGA
CAGGGACGGAAATGGTGGAGAAGCGACCCCTGCGGGAGAGGCACCGGACCCCTCCC
CAATGGCATCTCGCCTACGTGGGAGACGGGAGCCCCCTGTGGCCGACTCCACTACCT
CGCAGGCATTCCCCCTCCGCGCAGGAGGAAACGGACAGCTTCAATACTGGCCGTTCT
CCTCTTCTGACCTTTACAACTGGAAAAATAATAACCCTTCTTTTTCTGAAGATCCAGG
TAAACTGACAGCTCTGATCGAGTCTGTTCTCATCACCCATCAGCCCACCTGGGACGAC
TGTCAGCAGCTGTTGGGGACTCTGCTGACCGGAGAAGAAAAACAACGGGTGCTCTTA
GAGGCTAGAAAGGCGGTGCGGGGCGATGATGGGCGCCCCACTCAACTGCCCAATGA
AGTCGATGCCGCTTTTCCCCTCGAGCGCCCAGACTGGGATTACACCACCCAGGCAGG
TAGGAACCACCTAGTCCACTATCGCCAGTTGCTCCTAGCGGGTCTCCAAAACGCGGG
CAGAAGCCCCACCAATTTGGCCAAGGTAAAAGGAATAACACAAGGGCCCAATGAGT
CTCCCTCGGCCTTCCTAGAGAGACTTAAGGAAGCCTATCGCAGGTACACTCCTTATGA
CCCTGAGGACCCAGGGCAAGAAACTAATGTGTCTATGTCTTTCATTTGGCAGTCTGCC
CCAGACATTGGGAGAAAGTTAGAGAGGTTAGAAGATTTAAAAAACAAGACGCTTGG
AGATTTGGTTAGAGAGGCAGAAAAGATCTTTAATAAACGAGAAACCCCGGAAGAAA
GAGAGGAACGTATCAGGAGAGAAACAGAGGAAAAAGAAGAACGCCGTAGGACAGA
GGATGAGCAGAAAGAGAAAGAAAGAGATCGTAGGAGACATAGAGAGATGAGCAAG
CTATTGGCCACTGTCGTTAGTGGACAGAAACAGGATAGACAGGGAGGAGAACGAAG
GAGGTCCCAACTCGATCGCGACCAGTGTGCCTACTGCAAAGAAAAGGGGCACTGGGC
TAAAGATTGTCCCAAGAAACCACGAGGACCTCGGGGACCAAGACCCCAGACCTCCCT
CCTGACCCTAGATGAC
SEQ ID NO: 3 is a Codon Optimized MMLV-Gag Nucleotide Sequence
ATGGGACAGACCGTCACAACACCCCTGAGCCTGACCCTGGGACATTGGAAAGACGTG
GAGAGGATCGCACATAACCAGAGCGTGGACGTGAAGAAACGGAGATGGGTCACATT
CTGCAGTGCTGAGTGGCCAACTTTTAATGTGGGATGGCCCCGAGACGGCACTTTCAA
CAGGGATCTGATCACCCAGGTGAAGATCAAGGTCTTTAGCCCAGGACCTCACGGACA
TCCAGACCAGGTGCCTTATATCGTCACCTGGGAGGCACTGGCCTTCGATCCCCCTCCA
TGGGTGAAGCCATTTGTCCACCCAAAACCACCTCCACCACTGCCTCCAAGTGCCCCTT
CACTGCCACTGGAACCACCCCGGAGCACACCACCCCGCAGCTCCCTGTATCCTGCTCT
GACTCCATCTCTGGGCGCAAAGCCAAAACCACAGGTGCTGAGCGACTCCGGAGGACC
ACTGATTGACCTGCTGACAGAGGACCCCCCACCATACCGAGATCCTCGGCCTCCACC
AAGCGACCGCGATGGAAATGGAGGAGAGGCTACTCCTGCCGGCGAAGCCCCTGACC
CATCTCCAATGGCTAGTAGGCTGCGCGGCAGGCGCGAGCCTCCAGTGGCAGATAGCA
CCACATCCCAGGCCTTCCCTCTGAGGGCTGGGGGAAATGGGCAGCTCCAGTATTGGC
CATTTTCTAGTTCAGACCTGTACAACTGGAAGAACAATAACCCCTCTTTCAGTGAGGA
CCCCGGCAAACTGACCGCCCTGATCGAATCCGTGCTGATTACCCATCAGCCCACATG
GGACGATTGTCAGCAGCTCCTGGGCACCCTGCTGACCGGAGAGGAAAAGCAGCGCGT
GCTGCTGGAGGCTCGCAAAGCAGTCCGAGGGGACGATGGACGGCCCACACAGCTCCC
TAATGAGGTGGACGCCGCTTTTCCACTGGAAAGACCCGACTGGGATTATACTACCCA
GGCAGGGAGAAACCACCTGGTCCATTACAGGCAGCTCCTGCTGGCAGGCCTGCAGAA
TGCCGGGAGATCCCCCACCAACCTGGCCAAGGTGAAAGGCATCACACAGGGGCCTAA
TGAGTCACCAAGCGCCTTTCTGGAGAGGCTGAAGGAAGCTTACCGACGGTATACCCC
ATACGACCCTGAGGACCCCGGACAGGAAACAAACGTCTCCATGTCTTTCATCTGGCA
GTCTGCCCCAGACATTGGGCGGAAGCTGGAGAGACTGGAAGACCTGAAGAACAAGA
CCCTGGGCGACCTGGTGCGGGAGGCTGAAAAGATCTTCAACAAACGGGAGACCCCCG
AGGAAAGAGAGGAAAGGATTAGAAGGGAAACTGAGGAAAAGGAGGAACGCCGACG
GACCGAGGACGAACAGAAGGAGAAAGAACGAGATCGGCGGCGGCACCGGGAGATG
TCAAAGCTGCTGGCCACCGTGGTCAGCGGACAGAAACAGGACAGACAGGGAGGAGA
GCGACGGAGAAGCCAGCTCGACAGGGATCAGTGCGCATACTGTAAGGAAAAAGGCC
ATTGGGCCAAGGATTGCCCCAAAAAGCCAAGAGGACCAAGAGGACCAAGACCACAG
ACATCACTGCTGACCCTGGACGAC
SEQ ID NO: 4 is a SARS-CoV-2 Spike Glycoprotein, Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPRRARSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKLHYT
SEQ ID NO: 5 is a SARS-CoV-2 Spike Glycoprotein, Nucleotide Sequence
(Wuhan-Hu-1: Genbank Ref: MN908947)
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGTC
TAACATAATAAGAGGCTGGATTTTTGGTACTACTTTAGATTCGAAGACCCAGTCCCTA
CTTATTGTTAATAACGCTACTAATGTTGTTATTAAAGTCTGTGAATTTCAATTTTGTAA
TGATCCATTTTTGGGTGTTTATTACCACAAAAACAACAAAAGTTGGATGGAAAGTGA
GTTCAGAGTTTATTCTAGTGCGAATAATTGCACTTTTGAATATGTCTCTCAGCCTTTTC
TTATGGACCTTGAAGGAAAACAGGGTAATTTCAAAAATCTTAGGGAATTTGTGTTTA
AGAATATTGATGGTTATTTTAAAATATATTCTAAGCACACGCCTATTAATTTAGTGCG
TGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTG
ATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGT
GCACTTGACCCTCTCTCAGAAACAAAGTGTACGTTGAAATCCTTCACTGTAGAAAAA
GGAATCTATCAAACTTCTAACTTTAGAGTCCAACCAACAGAATCTATTGTTAGATTTC
CTAATATTACAAACTTGTGCCCTTTTGGTGAAGTTTTTAACGCCACCAGATTTGCATCT
GTTTATGCTTGGAACAGGAAGAGAATCAGCAACTGTGTTGCTGATTATTCTGTCCTAT
ATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTACCAG
ATGATTTTACAGGCTGCGTTATAGCTTGGAATTCTAACAATCTTGATTCTAAGGTTGG
TGGTAATTATAATTACCTGTATAGATTGTTTAGGAAGTCTAATCTCAAACCTTTTGAG
AGAGATATTTCAACTGAAATCTATCAGGCCGGTAGCACACCTTGTAATGGTGTTGAA
GGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACT
GTTTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCCTTT
CCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAG
TCCCTGTTGCTATTCATGCAGATCAACTTACTCCTACTTGGCGTGTTTATTCTACAGGT
TCTAATGTTTTTCAAACACGTGCAGGCTGTTTAATAGGGGCTGAACATGTCAACAACT
CATATGAGTGTGACATACCCATTGGTGCAGGTATATGCGCTAGTTATCAGACTCAGAC
TAATTCTCCTCGGCGGGCACGTAGTGTAGCTAGTCAATCCATCATTGCCTACACTATG
TCACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCAGT
AGATTGTACAATGTACATTTGTGGTGATTCAACTGAATGCAGCAATCTTTTGTTGCAA
TATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAG
CAAGAGGTCATTTATTGAAGATCTACTTTTCAACAAAGTGACACTTGCAGATGCTGGC
TTCATCAAACAATATGGTGATTGCCTTGGTGATATTGCTGCTAGAGACCTCATTTGTG
CACAAAAGTTTAACGGCCTTACTGTTTTGCCACCTTTGCTCACAGATGAAATGATTGC
TCAATACACTTCTGCACTGTTAGCGGGTACAATCACTTCTGGTTGGACCTTTGGTGCA
GGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAAACT
TCAAGATGTGGTCAACCAAAATGCACAAGCTTTAAACACGCTTGTTAAACAACTTAG
CTCCAATTTTGGTGCAATTTCAAGTGTTTTAAATGATATCCTTTCACGTCTTGACAAAG
TTGAGGCTGAAGTGCAAATTGATAGGTTGATCACAGGCAGACTTCAAAGTTTGCAGA
CATATGTGACTCAACAATTAATTAGAGCTGCAGAAATCAGAGCTTCTGCTAATCTTGC
TGCTACTAAAATGTCAGAGTGTGTACTTGGACAATCAAAAAGAGTTGATTTTTGTGGA
AAGGGCTATCATCTTATGTCCTTCCCTCAGTCAGCACCTCATGGTGTAGTCTTCTTGCA
TGTGACTTATGTCCCTGCACAAGAAAAGAACTTCACAACTGCTCCTGCCATTTGTCAT
GATGGAAAAGCACACTTTCCTCGTGAAGGTGTCTTTGTTTCAAATGGCACACACTGGT
TTGTAACACAAAGGAATTTTTATGAACCACAAATCATTACTACAGACAACACATTTGT
GTCTGGTAACTGTGATGTTGTAATAGGAATTGTCAACAACACAGTTTATGATCCTTTG
CAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTC
AAAAAGAAATTGACCGCCTCAATGAGGTTGCCAAGAATTTAAATGAATCTCTCATCG
ATCTCCAAGAACTTGGAAAGTATGAGCAGTATATAAAATGGCCATGGTACATTTGGC
TAGGTTTTATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTGCAAATTT
GATGAAGACGACTCTGAGCCAGTGCTCAAAGGAGTCAAATTACATTACACATAA
SEQ ID NO: 6 is a SARS-CoV-2 Spike Glycoprotein, Nucleotide Sequence,
Codon Optimized For Expression In Human Cell
ATGTTCGTGTTTCTGGTGCTGCTGCCTCTGGTGAGCTCCCAGTGCGTGAACCTGACCA
CAAGGACCCAGCTCCCCCCTGCCTATACCAATTCCTTCACACGGGGCGTGTACTATCC
CGACAAGGTGTTTAGATCTAGCGTGCTGCACTCCACACAGGATCTGTTTCTGCCTTTC
TTTTCTAACGTGACCTGGTTCCACGCCATCCATGTGAGCGGCACCAATGGCACAAAGC
GGTTCGACAATCCAGTGCTGCCCTTTAACGATGGCGTGTACTTCGCCTCCACCGAGAA
GTCTAACATCATCAGAGGCTGGATCTTTGGCACCACACTGGACAGCAAGACACAGTC
CCTGCTGATCGTGAACAATGCCACCAACGTGGTCATCAAGGTGTGCGAGTTCCAGTTT
TGTAATGATCCATTCCTGGGCGTGTACTATCACAAGAACAATAAGTCTTGGATGGAG
AGCGAGTTTCGCGTGTATTCCTCTGCCAACAATTGCACATTTGAGTACGTGTCCCAGC
CCTTCCTGATGGACCTGGAGGGCAAGCAGGGCAATTTCAAGAACCTGAGGGAGTTCG
TGTTTAAGAATATCGATGGCTACTTCAAGATCTACTCCAAGCACACCCCAATCAACCT
GGTGCGCGACCTGCCACAGGGCTTCTCTGCCCTGGAGCCACTGGTGGATCTGCCCATC
GGCATCAACATCACCCGGTTTCAGACACTGCTGGCCCTGCACAGAAGCTACCTGACA
CCAGGCGACAGCTCCTCTGGATGGACCGCCGGGGCCGCCGCCTACTATGTGGGCTAT
CTGCAGCCCAGGACCTTCCTGCTGAAGTACAACGAGAATGGCACCATCACAGACGCA
GTGGATTGCGCCCTGGACCCCCTGTCTGAGACCAAGTGTACACTGAAGAGCTTTACC
GTGGAGAAGGGCATCTATCAGACAAGCAATTTCAGGGTGCAGCCTACCGAGTCCATC
GTGCGCTTTCCCAATATCACAAACCTGTGCCCTTTTGGCGAGGTGTTCAACGCAACCA
GGTTCGCCAGCGTGTACGCATGGAATAGGAAGCGCATCTCCAACTGCGTGGCCGACT
ATTCTGTGCTGTACAACAGCGCCTCCTTCTCTACCTTTAAGTGCTATGGCGTGAGCCC
CACAAAGCTGAATGACCTGTGCTTTACCAACGTGTACGCCGATTCCTTCGTGATCAGG
GGCGACGAGGTGCGCCAGATCGCACCAGGACAGACAGGCAAGATCGCAGACTACAA
TTATAAGCTGCCTGACGATTTCACCGGCTGCGTGATCGCCTGGAACTCTAACAATCTG
GATAGCAAAGTGGGCGGCAACTACAATTATCTGTACCGGCTGTTTAGAAAGTCTAAT
CTGAAGCCATTCGAGAGGGACATCTCCACAGAGATCTACCAGGCCGGCTCTACCCCC
TGCAATGGCGTGGAGGGCTTTAACTGTTATTTCCCTCTGCAGAGCTACGGCTTCCAGC
CAACAAACGGCGTGGGCTATCAGCCCTACCGCGTGGTGGTGCTGTCTTTTGAGCTGCT
GCACGCACCTGCAACAGTGTGCGGACCAAAGAAGAGCACCAATCTGGTGAAGAACA
AGTGCGTGAACTTCAACTTCAACGGCCTGACCGGCACAGGCGTGCTGACCGAGTCCA
ACAAGAAGTTCCTGCCTTTTCAGCAGTTCGGCAGGGACATCGCAGATACCACAGACG
CCGTGCGCGACCCTCAGACCCTGGAGATCCTGGACATCACACCATGCTCCTTCGGCG
GCGTGTCTGTGATCACACCAGGCACCAATACAAGCAACCAGGTGGCCGTGCTGTATC
AGGACGTGAATTGTACCGAGGTGCCCGTGGCCATCCACGCAGATCAGCTCACCCCTA
CATGGCGGGTGTACTCTACCGGCAGCAACGTGTTCCAGACAAGAGCCGGCTGCCTGA
TCGGAGCCGAGCATGTGAACAATAGCTATGAGTGCGACATCCCTATCGGAGCCGGCA
TCTGTGCCTCCTACCAGACCCAGACAAACTCCCCACGGAGAGCCCGGTCTGTGGCCA
GCCAGTCCATCATCGCCTATACCATGAGCCTGGGGGCCGAGAACAGCGTGGCCTACT
CCAACAATTCTATCGCCATCCCTACCAACTTCACAATCTCCGTGACCACAGAGATCCT
GCCAGTGAGCATGACCAAGACATCCGTGGACTGCACAATGTATATCTGTGGCGATTC
CACCGAGTGCTCTAACCTGCTGCTGCAGTACGGCTCTTTTTGTACCCAGCTCAACAGA
GCCCTGACAGGCATCGCCGTGGAGCAGGACAAGAACACACAGGAGGTGTTCGCCCA
GGTGAAGCAGATCTACAAGACCCCACCCATCAAGGACTTTGGCGGCTTCAACTTCAG
CCAGATCCTGCCCGATCCTAGCAAGCCATCCAAGCGGTCTTTTATCGAGGACCTGCTG
TTCAACAAGGTGACCCTGGCCGATGCCGGCTTCATCAAGCAGTATGGCGATTGCCTG
GGCGACATCGCCGCCAGAGACCTGATCTGTGCCCAGAAGTTTAATGGCCTGACCGTG
CTGCCTCCACTGCTGACAGATGAGATGATCGCCCAGTACACATCTGCCCTGCTGGCCG
GCACCATCACAAGCGGATGGACCTTCGGGGCCGGGGCCGCCCTGCAGATCCCCTTTG
CCATGCAGATGGCCTATCGGTTCAACGGCATCGGCGTGACCCAGAATGTGCTGTACG
AGAACCAGAAGCTGATCGCCAATCAGTTTAACTCCGCCATCGGCAAGATCCAGGACT
CTCTGAGCTCCACAGCCAGCGCCCTGGGCAAGCTGCAGGATGTGGTGAATCAGAACG
CCCAGGCCCTGAATACCCTGGTGAAGCAGCTCAGCAGCAACTTCGGGGCCATCAGCA
GCGTGCTGAACGACATCCTGAGCCGGCTGGACAAGGTGGAGGCAGAGGTGCAGATC
GACCGGCTGATCACAGGCAGACTGCAGTCCCTGCAGACCTACGTGACACAGCAGCTC
ATCAGGGCCGCCGAGATCAGGGCCTCTGCCAATCTGGCCGCCACCAAGATGAGCGAG
TGCGTGCTGGGCCAGTCCAAGAGAGTGGACTTTTGTGGCAAGGGCTATCACCTGATG
AGCTTCCCACAGTCCGCCCCTCACGGAGTGGTGTTTCTGCATGTGACCTACGTGCCAG
CCCAGGAGAAGAACTTCACCACAGCCCCCGCAATCTGCCACGATGGCAAGGCACACT
TTCCCCGGGAGGGCGTGTTCGTGAGCAACGGCACCCACTGGTTTGTGACACAGCGCA
ATTTCTACGAGCCACAGATCATCACCACAGACAATACATTCGTGTCCGGCAACTGTG
ACGTGGTCATCGGCATCGTGAACAATACCGTGTATGATCCTCTGCAGCCAGAGCTGG
ACTCTTTTAAGGAGGAGCTGGATAAGTACTTCAAGAATCACACCAGCCCCGACGTGG
ATCTGGGCGACATCTCTGGCATCAATGCCAGCGTGGTGAACATCCAGAAGGAGATCG
ACAGGCTGAACGAGGTGGCCAAGAATCTGAACGAGTCCCTGATCGATCTGCAGGAGC
TGGGCAAGTATGAGCAGTACATCAAGTGGCCCTGGTATATCTGGCTGGGCTTCATCG
CCGGCCTGATCGCCATCGTGATGGTGACCATCATGCTGTGCTGTATGACAAGCTGCTG
TTCCTGCCTGAAGGGCTGCTGTTCTTGTGGCTCCTGCTGTAAGTTTGATGAGGACGAT
AGCGAGCCTGTGCTGAAGGGCGTGAAGCTGCACTACACCTGA
SEQ ID NO: 7 is a SARS-CoV Spike Glycoprotein, Amino Acid Sequence
(HKU-39849, Genbank Ref: JN854286.1)
MFIFLLFLTLTSGSDLDRCTTFDDVQAPNYTQHTSSMRGVYYPDEIFRSDTLYLTQDLFLP
FYSNVTGFHTINHTFGNPVIPFKDGIYFAATEKSNVVRGWVFGSTMNNKSQSVIIINNSTN
VVIRACNFELCDNPFFAVSKPMGTQTHTMIFDNAFNCTFEYISDAFSLDVSEKSGNFKHLR
EFVFKNKDGFLYVYKGYQPIDVVRDLPSGFNTLKPIFKLPLGINITNFRAILTAFSPAQDIW
GTSAAAYFVGYLKPTTFMLKYDENGTITDAVDCSQNPLAELKCSVKSFEIDKGIYQTSNF
RVVPSGDVVRFPNITNLCPFGEVFNATKFPSVYAWERKKISNCVADYSVLYNSTFFSTFKC
YGVSATKLNDLCFSNVYADSFVVKGDDVRQIAPGQTGVIADYNYKLPDDFMGCVLAWN
TRNIDATSTGNYNYKYRYLRHGKLRPFERDISNVPFSPDGKPCTPPALNCYWPLNDYGFY
TTTGIGYQPYRVVVLSFELLNAPATVCGPKLSTDLIKNQCVNFNFNGLTGTGVLTPSSKRF
QPFQQFGRDVSDFTDSVRDPKTSEILDISPCSFGGVSVITPGTNASSEVAVLYQDVNCTDV
STAIHADQLTPAWRIYSTGNNVFQTQAGCLIGAEHVDTSYECDIPIGAGICASYHTVSLLR
STSQKSIVAYTMSLGADSSIAYSNNTIAIPTNFSISITTEVMPVSMAKTSVDCNMYICGDST
ECANLLLQYGSFCTQLNRALSGIAAEQDRNTREVFAQVKQMYKTPTLKYFGGFNFSQILP
DPLKPTKRSFIEDLLFNKVTLADAGFMKQYGECLGDINARDLICAQKFNGLTVLPPLLTD
DMIAAYTAALVSGTATAGWTFGAGAALQIPFAMQMAYRFNGIGVTQNVLYENQKQIAN
QFNKAISQIQESLTTTSTALGKLQDVVNQNAQALNTLVKQLSSNFGAISSVLNDILSRLDK
VEAEVQIDRLITGRLQSLQTYVTQQLIRAAEIRASANLAATKMSECVLGQSKRVDFCGKG
YHLMSFPQAAPHGVVFLHVTYVPSQERNFTTAPAICHEGKAYFPREGVFVFNGTSWFITQ
RNFFSPQIITTDNTFVSGNCDVVIGIINNTVYDPLQPELDSFKEELDKYFKNHTSPDVDLGD
ISGINASVVNIQKEIDRLNEVAKNLNESLIDLQELGKYEQYIKWPWYVWLGFIAGLIAIVM
VTILLCCMTSCCSCLKGACSCGSCCKFDEDDSEPVLKGVKLHYT
SEQ ID NO: 8 is a SARS-CoV Spike Glycoprotein, Nucleotide Sequence
ATGTTTATTTTCTTATTATTTCTTACTCTCACTAGTGGTAGTGACCTTGACCGGTGCAC
CACTTTTGATGATGTTCAAGCTCCTAATTACACTCAACATACTTCATCTATGAGGGGG
GTTTACTATCCTGATGAAATTTTTAGATCAGACACTCTTTATTTAACTCAGGATTTATT
TCTTCCATTTTATTCTAATGTTACAGGGTTTCATACTATTAATCATACGTTTGGCAACC
CTGTCATACCTTTTAAGGATGGTATTTATTTTGCTGCCACAGAGAAATCAAATGTTGT
CCGTGGTTGGGTTTTTGGTTCTACCATGAACAACAAGTCACAGTCGGTGATTATTATT
AACAATTCTACTAATGTTGTTATACGAGCATGTAACTTTGAATTGTGTGACAACCCTT
TCTTTGCTGTTTCTAAACCCATGGGTACACAGACACATACTATGATATTCGATAATGC
ATTTAATTGCACTTTCGAGTACATATCTGATGCCTTTTCGCTTGATGTTTCAGAAAAGT
CAGGTAATTTTAAACACTTACGAGAGTTTGTGTTTAAAAATAAAGATGGGTTTCTCTA
TGTTTATAAGGGCTATCAACCTATAGATGTAGTTCGTGATCTACCTTCTGGTTTTAAC
ACTTTGAAACCTATTTTTAAGTTGCCTCTTGGTATTAACATTACAAATTTTAGAGCCAT
TCTTACAGCCTTTTCACCTGCTCAAGACATTTGGGGCACGTCAGCTGCAGCCTATTTT
GTTGGCTATTTAAAGCCAACTACATTTATGCTCAAGTATGATGAAAATGGTACAATCA
CAGATGCTGTTGATTGTTCTCAAAATCCACTTGCTGAACTCAAATGCTCTGTTAAGAG
CTTTGAGATTGACAAAGGAATTTACCAGACCTCTAATTTCAGGGTTGTTCCCTCAGGA
GATGTTGTGAGATTCCCTAATATTACAAACTTGTGTCCTTTTGGAGAGGTTTTTAATG
CTACTAAATTCCCTTCTGTCTATGCATGGGAGAGAAAAAAAATTTCTAATTGTGTTGC
TGATTACTCTGTGCTCTACAACTCAACATTTTTTTCAACCTTTAAGTGCTATGGCGTTT
CTGCCACTAAGTTGAATGATCTTTGCTTCTCCAATGTCTATGCAGATTCTTTTGTAGTC
AAGGGAGATGATGTAAGACAAATAGCGCCAGGACAAACTGGTGTTATTGCTGATTAT
AATTATAAATTGCCAGATGATTTCATGGGTTGTGTCCTTGCTTGGAATACTAGGAACA
TTGATGCTACTTCAACTGGTAATTATAATTATAAATATAGGTATCTTAGACATGGCAA
GCTTAGGCCCTTTGAGAGAGACATATCTAATGTGCCTTTCTCCCCTGATGGCAAACCT
TGCACCCCACCTGCTCTTAATTGTTATTGGCCATTAAATGATTATGGTTTTTACACCAC
TACTGGCATTGGCTACCAACCTTACAGAGTTGTAGTACTTTCTTTTGAACTTTTAAATG
CACCGGCCACGGTTTGTGGACCAAAATTATCCACTGACCTTATTAAGAACCAGTGTGT
CAATTTTAATTTTAATGGACTCACTGGTACTGGTGTGTTAACTCCTTCTTCAAAGAGA
TTTCAACCATTTCAACAATTTGGCCGTGATGTTTCTGATTTCACTGATTCCGTTCGAGA
TCCTAAAACATCTGAAATATTAGACATTTCACCTTGCTCTTTTGGGGGTGTAAGTGTA
ATTACACCTGGAACAAATGCTTCATCTGAAGTTGCTGTTCTATATCAAGATGTTAACT
GCACTGATGTTTCTACAGCAATTCATGCAGATCAACTCACACCAGCTTGGCGCATATA
TTCTACTGGAAACAATGTATTCCAGACTCAAGCAGGCTGTCTTATAGGAGCTGAGCAT
GTCGACACTTCTTATGAGTGCGACATTCCTATTGGAGCTGGCATTTGTGCTAGTTACC
ATACAGTTTCTTTATTACGTAGTACTAGCCAAAAATCTATTGTGGCTTATACTATGTCT
TTAGGTGCTGATAGTTCAATTGCTTACTCTAATAACACCATTGCTATACCTACTAACTT
TTCAATTAGCATTACTACAGAAGTAATGCCTGTTTCTATGGCTAAAACCTCCGTAGAT
TGTAATATGTACATCTGCGGAGATTCTACTGAATGTGCTAATTTGCTTCTCCAATATG
GTAGCTTTTGCACACAACTAAATCGTGCACTCTCAGGTATTGCTGCTGAACAGGATCG
CAACACACGTGAAGTGTTCGCTCAAGTCAAACAAATGTACAAAACCCCAACTTTGAA
ATATTTTGGTGGTTTTAATTTTTCACAAATATTACCTGACCCTCTAAAGCCAACTAAG
AGGTCTTTTATTGAGGACTTGCTCTTTAATAAGGTGACACTCGCTGATGCTGGCTTCA
TGAAGCAATATGGCGAATGCCTAGGTGATATTAATGCTAGAGATCTCATTTGTGCGC
AGAAGTTCAATGGACTTACAGTGTTGCCACCTCTGCTCACTGATGATATGATTGCTGC
CTACACTGCTGCTCTAGTTAGTGGTACTGCCACTGCTGGATGGACATTTGGTGCTGGC
GCTGCTCTTCAAATACCTTTTGCTATGCAAATGGCATATAGGTTCAATGGCATTGGAG
TTACCCAAAATGTTCTCTATGAGAACCAAAAACAAATCGCCAACCAATTTAACAAGG
CGATTAGTCAAATTCAAGAATCACTTACAACAACATCAACTGCATTGGGCAAGCTGC
AAGACGTTGTTAACCAGAATGCTCAAGCATTAAACACACTTGTTAAACAACTTAGCT
CTAATTTTGGTGCAATTTCAAGTGTGCTAAATGATATCCTTTCGCGACTTGATAAAGT
CGAGGCGGAGGTACAAATTGACAGGTTAATTACAGGCAGACTTCAAAGCCTTCAAAC
CTATGTAACACAACAACTAATCAGGGCTGCTGAAATCAGGGCTTCTGCTAATCTTGCT
GCTACTAAAATGTCTGAGTGTGTTCTTGGACAATCAAAAAGAGTTGACTTTTGTGGAA
AGGGCTACCACCTTATGTCCTTCCCACAAGCAGCCCCGCATGGTGTTGTCTTCCTACA
TGTCACGTATGTGCCATCCCAGGAGAGGAACTTCACCACAGCGCCAGCAATTTGTCA
TGAAGGCAAAGCATACTTCCCTCGTGAAGGTGTTTTTGTGTTTAATGGCACTTCTTGG
TTTATTACACAGAGGAACTTCTTTTCTCCACAAATAATTACTACAGACAATACATTTG
TCTCAGGAAATTGTGATGTCGTTATTGGCATCATTAACAACACAGTTTATGATCCTCT
GCAACCTGAGCTTGACTCATTCAAAGAAGAGCTGGACAAGTACTTCAAAAATCATAC
ATCACCAGATGTTGATCTTGGCGACATTTCAGGCATTAACGCTTCTGTCGTCAACATT
CAAAAAGAAATTGACCGCCTCAATGAGGTCGCTAAAAATTTAAATGAATCACTCATT
GACCTTCAAGAATTGGGAAAATATGAGCAATATATTAAATGGCCTTGGTATGTTTGG
CTCGGCTTCATTGCTGGACTAATTGCCATCGTCATGGTTACAATCTTGCTTTGTTGCAT
GACTAGTTGTTGCAGTTGCCTCAAGGGTGCATGCTCTTGTGGTTCTTGCTGCAAGTTT
GATGAGGATGACTCTGAGCCAGTTCTCAAGGGTGTCAAATTACATTACACATAA
SEQ ID NO: 9 is a SARS-CoV Spike Glycoprotein, Nucleotide Sequence,
Codon Optimized for Expression In Human Cells
ATGTTCATCTTTCTGCTGTTCCTGACCCTGACAAGCGGCTCCGACCTGGATAGGTGCA
CCACATTTGACGATGTGCAGGCCCCCAACTACACACAGCACACCAGCTCCATGAGGG
GCGTGTACTATCCTGATGAGATCTTCCGCTCTGACACACTGTACCTGACCCAGGACCT
GTTCCTGCCTTTTTATAGCAATGTGACAGGCTTCCACACCATCAATCACACATTTGGC
AACCCCGTGATCCCTTTCAAGGATGGCATCTACTTTGCCGCCACCGAGAAGTCTAACG
TGGTGCGGGGATGGGTGTTCGGCAGCACAATGAACAATAAGTCTCAGAGCGTGATCA
TCATCAACAATAGCACCAACGTGGTCATCAGAGCCTGCAATTTTGAGCTGTGCGACA
ACCCCTTCTTTGCCGTGTCCAAGCCTATGGGCACCCAGACACACACCATGATCTTTGA
TAATGCCTTCAACTGTACCTTTGAGTACATCAGCGATGCCTTTTCCCTGGACGTGTCT
GAGAAGTCCGGCAACTTCAAGCACCTGAGGGAGTTCGTGTTTAAGAATAAGGACGGC
TTCCTGTACGTGTATAAGGGCTATCAGCCCATCGATGTGGTGCGCGACCTGCCTTCCG
GCTTCAACACCCTGAAGCCAATCTTTAAGCTGCCCCTGGGCATCAATATCACCAACTT
CAGGGCCATCCTGACAGCCTTTAGCCCAGCACAGGACATCTGGGGCACCAGCGCCGC
CGCCTACTTCGTGGGCTATCTGAAGCCCACCACCTTCATGCTGAAGTACGATGAGAAC
GGCACAATCACCGACGCCGTGGATTGCAGCCAGAATCCACTGGCCGAGCTGAAGTGT
TCCGTGAAGTCTTTCGAGATCGACAAGGGCATCTATCAGACCTCCAACTTTAGGGTGG
TGCCATCTGGCGATGTGGTGCGCTTCCCAAATATCACCAACCTGTGCCCCTTCGGCGA
GGTGTTTAATGCCACAAAGTTCCCCAGCGTGTACGCCTGGGAGCGCAAGAAGATCAG
CAACTGCGTGGCCGACTACTCCGTGCTGTATAATAGCACCTTCTTCAGCACCTTCAAG
TGCTACGGCGTGAGCGCCACCAAGCTGAATGACCTGTGCTTCTCTAACGTGTATGCCG
ATAGCTTTGTGGTGAAGGGCGACGATGTGAGGCAGATCGCACCTGGACAGACCGGCG
TGATCGCAGACTACAACTATAAGCTGCCAGACGATTTCATGGGCTGCGTGCTGGCCT
GGAATACACGCAACATCGATGCCACATCCACCGGCAACTACAATTATAAGTACCGGT
ATCTGAGACACGGCAAGCTGCGGCCCTTCGAGAGAGACATCTCCAATGTGCCATTTT
CTCCAGATGGCAAGCCATGCACCCCACCTGCCCTGAATTGTTACTGGCCTCTGAACGA
CTACGGCTTCTATACCACAACCGGCATCGGCTACCAGCCTTATAGGGTGGTGGTGCTG
TCCTTTGAGCTGCTGAACGCACCTGCAACCGTGTGCGGACCAAAGCTGTCTACAGATC
TGATCAAGAATCAGTGCGTGAACTTCAACTTCAACGGCCTGACAGGCACCGGCGTGC
TGACCCCTTCTAGCAAGCGGTTCCAGCCATTTCAGCAGTTCGGCAGAGACGTGAGCG
ATTTCACCGACTCCGTGCGCGACCCAAAGACATCCGAGATCCTGGACATCAGCCCCT
GCTCCTTTGGCGGCGTGTCTGTGATCACACCTGGCACCAACGCCTCCTCTGAGGTGGC
CGTGCTGTACCAGGATGTGAATTGTACCGACGTGAGCACAGCAATCCACGCAGACCA
GCTCACCCCAGCATGGCGGATCTATTCCACCGGCAACAACGTGTTCCAGACACAGGC
AGGATGCCTGATCGGAGCCGAGCATGTGGATACAAGCTACGAGTGCGACATCCCCAT
CGGAGCCGGCATCTGTGCCTCTTATCACACCGTGAGCCTGCTGAGATCCACATCTCAG
AAGTCTATCGTGGCCTACACCATGAGCCTGGGGGCCGATAGCTCCATCGCCTATTCCA
ACAATACCATCGCCATCCCAACAAACTTCAGCATCTCCATCACAACCGAAGTGATGC
CCGTGTCCATGGCCAAGACCTCTGTGGACTGCAACATGTACATCTGTGGCGATAGCA
CAGAGTGCGCCAATCTGCTGCTGCAGTATGGCTCCTTTTGTACCCAGCTCAACCGGGC
CCTGTCTGGAATCGCCGCCGAGCAGGACAGGAATACACGCGAGGTGTTCGCCCAGGT
GAAGCAGATGTACAAGACACCTACCCTGAAGTATTTTGGCGGCTTCAACTTTTCTCAG
ATCCTGCCTGATCCACTGAAGCCAACCAAGCGGAGCTTCATCGAGGACCTGCTGTTTA
ATAAGGTGACACTGGCCGATGCCGGCTTCATGAAGCAGTACGGCGAGTGCCTGGGCG
ACATCAACGCCAGAGACCTGATCTGTGCCCAGAAGTTTAATGGCCTGACCGTGCTGC
CACCCCTGCTGACAGACGATATGATCGCAGCATATACCGCCGCCCTGGTGTCCGGCA
CAGCCACCGCCGGCTGGACCTTCGGGGCCGGGGCCGCCCTGCAGATCCCTTTCGCCA
TGCAGATGGCCTACCGGTTTAACGGCATCGGCGTGACCCAGAATGTGCTGTATGAGA
ACCAGAAGCAGATCGCCAATCAGTTTAACAAGGCCATCAGCCAGATCCAGGAGTCCC
TGACAACCACATCTACCGCCCTGGGCAAGCTGCAGGACGTGGTGAATCAGAACGCCC
AGGCCCTGAATACACTGGTGAAGCAGCTCAGCAGCAACTTCGGGGCCATCAGCAGCG
TGCTGAACGACATCCTGAGCCGGCTGGACAAGGTGGAGGCAGAGGTGCAGATCGAT
AGGCTGATCACCGGCAGACTGCAGTCTCTGCAGACATACGTGACCCAGCAGCTCATC
AGGGCCGCCGAGATCAGAGCCAGCGCCAACCTGGCCGCCACAAAGATGTCCGAGTG
CGTGCTGGGCCAGTCTAAGAGGGTGGACTTCTGTGGCAAGGGCTACCACCTGATGTC
CTTTCCACAGGCCGCCCCTCACGGAGTGGTGTTCCTGCATGTGACCTATGTGCCTTCT
CAGGAGCGCAACTTTACCACAGCCCCAGCAATCTGCCACGAGGGCAAGGCATACTTC
CCCCGGGAGGGCGTGTTCGTGTTTAACGGCACCTCCTGGTTTATCACACAGAGAAATT
TCTTTTCCCCTCAGATCATCACCACAGACAATACCTTCGTGAGCGGCAACTGTGACGT
GGTCATCGGCATCATCAACAATACAGTGTACGATCCTCTGCAGCCAGAGCTGGACAG
CTTCAAGGAGGAGCTGGATAAGTACTTCAAGAACCACACCTCCCCCGACGTGGATCT
GGGCGACATCAGCGGCATCAATGCCTCCGTGGTGAACATCCAGAAGGAGATCGACAG
ACTGAATGAGGTGGCCAAGAATCTGAACGAGTCCCTGATCGATCTGCAGGAGCTGGG
CAAGTACGAGCAGTATATCAAGTGGCCATGGTACGTGTGGCTGGGCTTCATCGCCGG
CCTGATCGCCATCGTGATGGTGACCATCCTGCTGTGCTGTATGACATCTTGCTGTAGC
TGCCTGAAGGGAGCCTGCTCCTGTGGCTCTTGCTGTAAGTTTGACGAGGACGATAGC
GAGCCCGTGCTGAAGGGCGTGAAGCTGCACTATACCTGA
SEQ ID NO: 10 is a MERS-CoV Spike Glycoprotein, Amino Acid Sequence
MIHSVFLLMFLLTPTESYVDVGPDSVKSACIEVDIQQTFFDKTWPRPIDVSKADGITYPQGR
TYSNITITYQGLFPYQGDHGDMYVYSAGHATGTTPQKLFVANYSQDVKQFANGFVVRIG
AAANSTGTVIISPSTSATIRKIYPAFMLGSSVGNFSDGKMGRFFNHTLVLLPDGCGTLLRA
FYCILEPRSGNHCPAGNSYTSFATYHTPATDCSDGNYNRNASLNSFKEYFNLRNCTFMYT
YNITEDEILEWFGITQTAQGVHLFSSRYVDLYGGNMFQFATLPVYDTIKYYSIIPHSIRSIQS
DRKAWAAFYVYKLQPLTFLLDFSVDGYIRRAIDCGFNDLSQLHCSYESFDVESGVYSVSS
FEAKPSGSVVEQAEGVECDFSPLLSGTPPQVYNFKRLVFTNCNYNLTKLLSLFSVNDFTCS
QISPAAIASNCYSSLILDYFSYPLSMKSDLSVSSAGPISQFNYKQSFSNPTCLILATVPHNLT
TITKPLKYSYINKCSRLLSDDRTEVPQLVNANQYSPCVSIVPSTVWEDGDYYRKQLSPLEG
GGWLVASGSTVAMTEQLQMGFGITVQYGTDTNSVCPKLEFANDTKIASQLGNCVEYSLY
GVSGRGVFQNCTAVGVRQQRFVYDAYQNLVGYYSDDGNYYCLRACVSVPVSVIYDKET
KTHATLFGSVACEHISSTMSQYSRSTRSMLKRRDSTYGPLQTPVGCVLGLVNSSLFVEDC
KLPLGQSLCALPDTPSTLTPRSVRSVPGEMRLASIAFNHPIQVDQLNSSYFKLSIPTNFSFG
VTQEYIQTTIQKVTVDCKQYVCNGFQKCEQLLREYGQFCSKINQALHGANLRQDDSVRN
LFASVKSSQSSPIIPGFGGDFNLTLLEPVSISTGSRSARSAIEDLLFDKVTIADPGYMQGYDD
CMQQGPASARDLICAQYVAGYKVLPPLMDVNMEAAYTSSLLGSIAGVGWTAGLSSFAAI
PFAQSIFYRLNGVGITQQVLSENQKLIANKFNQALGAMQTGFTTTNEAFQKVQDAVNNN
AQALSKLASELSNTFGAISASIGDIIQRLDVLEQDAQIDRLINGRLTTLNAFVAQQLVRSES
AALSAQLAKDKVNECVKAQSKRSGFCGQGTHIVSFVVNAPNGLYFMHVGYYPSNHIEV
VSAYGLCDAANPTNCIAPVNGYFIKTNNTRIVDEWSYTGSSFYAPEPITSLNTKYVAPQVT
YQNISTNLPPPLLGNSTGIDFQDELDEFFKNVSTSIPNFGSLTQINTTLLDLTYEMLSLQQV
VKALNESYIDLKELGNYTYYNKWPWYIWLGFIAGLVALALCVFFILCCTGCGTNCMGKL
KCNRCCDRYEEYDLEPHKVHVH
SEQ ID NO: 11 is a MERS-CoV Spike Glycoprotein, Nucleotide Sequence
(EMC/2012, Genbank Ref: JX869059.2)
ATGATACACTCAGTGTTTCTACTGATGTTCTTGTTAACACCTACAGAAAGTTACGTTG
ATGTAGGGCCAGATTCTGTTAAGTCTGCTTGTATTGAGGTTGATATACAACAGACTTT
CTTTGATAAAACTTGGCCTAGGCCAATTGATGTTTCTAAGGCTGACGGTATTATATAC
CCTCAAGGCCGTACATATTCTAACATAACTATCACTTATCAAGGTCTTTTTCCCTATCA
GGGAGACCATGGTGATATGTATGTTTACTCTGCAGGACATGCTACAGGCACAACTCC
ACAAAAGTTGTTTGTAGCTAACTATTCTCAGGACGTCAAACAGTTTGCTAATGGGTTT
GTCGTCCGTATAGGAGCAGCTGCCAATTCCACTGGCACTGTTATTATTAGCCCATCTA
CCAGCGCTACTATACGAAAAATTTACCCTGCTTTTATGCTGGGTTCTTCAGTTGGTAA
TTTCTCAGATGGTAAAATGGGCCGCTTCTTCAATCATACTCTAGTTCTTTTGCCCGATG
GATGTGGCACTTTACTTAGAGCTTTTTATTGTATTCTAGAGCCTCGCTCTGGAAATCAT
TGTCCTGCTGGCAATTCCTATACTTCTTTTGCCACTTATCACACTCCTGCAACAGATTG
TTCTGATGGCAATTACAATCGTAATGCCAGTCTGAACTCTTTTAAGGAGTATTTTAAT
TTACGTAACTGCACCTTTATGTACACTTATAACATTACCGAAGATGAGATTTTAGAGT
GGTTTGGCATTACACAAACTGCTCAAGGTGTTCACCTCTTCTCATCTCGGTATGTTGA
TTTGTACGGCGGCAATATGTTTCAATTTGCCACCTTGCCTGTTTATGATACTATTAAGT
ATTATTCTATCATTCCTCACAGTATTCGTTCTATCCAAAGTGATAGAAAAGCTTGGGC
TGCCTTCTACGTATATAAACTTCAACCGTTAACTTTCCTGTTGGATTTTTCTGTTGATG
GTTATATACGCAGAGCTATAGACTGTGGTTTTAATGATTTGTCACAACTCCACTGCTC
ATATGAATCCTTCGATGTTGAATCTGGAGTTTATTCAGTTTCGTCTTTCGAAGCAAAA
CCTTCTGGCTCAGTTGTGGAACAGGCTGAAGGTGTTGAATGTGATTTTTCACCTCTTC
TGTCTGGCACACCTCCTCAGGTTTATAATTTCAAGCGTTTGGTTTTTACCAATTGCAAT
TATAATCTTACCAAATTGCTTTCACTTTTTTCTGTGAATGATTTTACTTGTAGTCAAAT
ATCTCCAGCAGCAATTGCTAGCAACTGTTATTCTTCACTGATTTTGGATTACTTTTCAT
ACCCACTTAGTATGAAATCCGATCTCAGTGTTAGTTCTGCTGGTCCAATATCCCAGTT
TAATTATAAACAGTCCTTTTCTAATCCCACATGTTTGATTTTAGCGACTGTTCCTCATA
ACCTTACTACTATTACTAAGCCTCTTAAGTACAGCTATATTAACAAGTGCTCTCGTCTT
CTTTCTGATGATCGTACTGAAGTACCTCAGTTAGTGAACGCTAATCAATACTCACCCT
GTGTATCCATTGTCCCATCCACTGTGTGGGAAGACGGTGATTATTATAGGAAACAACT
ATCTCCACTTGAAGGTGGTGGCTGGCTTGTTGCTAGTGGCTCAACTGTTGCCATGACT
GAGCAATTACAGATGGGCTTTGGTATTACAGTTCAATATGGTACAGACACCAATAGT
GTTTGCCCCAAGCTTGAATTTGCTAATGACACAAAAATTGCCTCTCAATTAGGCAATT
GCGTGGAATATTCCCTCTATGGTGTTTCGGGCCGTGGTGTTTTTCAGAATTGCACAGC
TGTAGGTGTTCGACAGCAGCGCTTTGTTTATGATGCGTACCAGAATTTAGTTGGCTAT
TATTCTGATGATGGCAACTACTACTGTTTGCGTGCTTGTGTTAGTGTTCCTGTTTCTGT
CATCTATGATAAAGAAACTAAAACCCACGCTACTCTATTTGGTAGTGTTGCATGTGAA
CACATTTCTTCTACCATGTCTCAATACTCCCGTTCTACGCGATCAATGCTTAAACGGC
GAGATTCTACATATGGCCCCCTTCAGACACCTGTTGGTTGTGTCCTAGGACTTGTTAA
TTCCTCTTTGTTCGTAGAGGACTGCAAGTTGCCTCTTGGTCAATCTCTCTGTGCTCTTC
CTGACACACCTAGTACTCTCACACCTCGCAGTGTGCGCTCTGTTCCAGGTGAAATGCG
CTTGGCATCCATTGCTTTTAATCATCCTATTCAGGTTGATCAACTTAATAGTAGTTATT
TTAAATTAAGTATACCCACTAATTTTTCCTTTGGTGTGACTCAGGAGTACATTCAGAC
AACCATTCAGAAAGTTACTGTTGATTGTAAACAGTACGTTTGCAATGGTTTCCAGAAG
TGTGAGCAATTACTGCGCGAGTATGGCCAGTTTTGTTCCAAAATAAACCAGGCTCTCC
ATGGTGCCAATTTACGCCAGGATGATTCTGTACGTAATTTGTTTGCGAGCGTGAAAAG
CTCTCAATCATCTCCTATCATACCAGGTTTTGGAGGTGACTTTAATTTGACACTTCTAG
AACCTGTTTCTATATCTACTGGCAGTCGTAGTGCACGTAGTGCTATTGAGGATTTGCT
ATTTGACAAAGTCACTATAGCTGATCCTGGTTATATGCAAGGTTACGATGATTGCATG
CAGCAAGGTCCAGCATCAGCTCGTGATCTTATTTGTGCTCAATATGTGGCTGGTTACA
AAGTATTACCTCCTCTTATGGATGTTAATATGGAAGCCGCGTATACTTCATCTTTGCTT
GGCAGCATAGCAGGTGTTGGCTGGACTGCTGGCTTATCCTCCTTTGCTGCTATTCCAT
TTGCACAGAGTATCTTTTATAGGTTAAACGGTGTTGGCATTACTCAACAGGTTCTTTC
AGAGAACCAAAAGCTTATTGCCAATAAGTTTAATCAGGCTCTGGGAGCTATGCAAAC
AGGCTTCACTACAACTAATGAAGCTTTTCAGAAGGTTCAGGATGCTGTGAACAACAA
TGCACAGGCTCTATCCAAATTAGCTAGCGAGCTATCTAATACTTTTGGTGCTATTTCC
GCCTCTATTGGAGACATCATACAACGTCTTGATGTTCTCGAACAGGACGCCCAAATA
GACAGACTTATTAATGGCCGTTTGACAACACTAAATGCTTTTGTTGCACAGCAGCTTG
TTCGTTCCGAATCAGCTGCTCTTTCCGCTCAATTGGCTAAAGATAAAGTCAATGAGTG
TGTCAAGGCACAATCCAAGCGTTCTGGATTTTGCGGTCAAGGCACACATATAGTGTCC
TTTGTTGTAAATGCCCCTAATGGCCTTTACTTCATGCATGTTGGTTATTACCCTAGCAA
CCACATTGAGGTTGTTTCTGCTTATGGTCTTTGCGATGCAGCTAACCCTACTAATTGTA
TAGCCCCTGTTAATGGCTACTTTATTAAAACTAATAACACTAGGATTGTTGATGAGTG
GTCATATACTGGCTCGTCCTTCTATGCACCTGAGCCCATTACCTCCCTTAATACTAAGT
ATGTTGCACCACAGGTGACATACCAAAACATTTCTACTAACCTCCCTCCTCCTCTTCT
CGGCAATTCCACCGGGATTGACTTCCAAGATGAGTTGGATGAGTTTTTCAAAAATGTT
AGCACCAGTATACCTAATTTTGGTTCCCTAACACAGATTAATACTACATTACTCGATC
TTACCTACGAGATGTTGTCTCTTCAACAAGTTGTTAAAGCCCTTAATGAGTCTTACAT
AGACCTTAAAGAGCTTGGCAATTATACTTATTACAACAAATGGCCGTGGTACATTTGG
CTTGGTTTCATTGCTGGGCTTGTTGCCTTAGCTCTATGCGTCTTCTTCATACTGTGCTG
CACTGGTTGTGGCACAAACTGTATGGGAAAACTTAAGTGTAATCGTTGTTGTGATAG
ATACGAGGAATACGACCTCGAGCCGCATAAGGTTCATGTTCACTAA
SEQ ID NO: 12 is a MERS-CoV Spike Glycoprotein, Nucleotide Sequence,
Codon Optimized For Expression In Human Cells
ATGATCCACAGCGTGTTCCTGCTGATGTTTCTGCTGACACCTACCGAGTCCTACGTGG
ATGTGGGCCCAGACTCTGTGAAGAGCGCCTGCATCGAGGTGGACATCCAGCAGACAT
TCTTTGACAAGACCTGGCCCAGACCCATCGACGTGAGCAAGGCAGACGGAATCATCT
ACCCACAGGGACGCACATATAGCAACATCACAATCACCTACCAGGGCCTGTTCCCTT
ATCAGGGCGACCACGGCGATATGTACGTGTATAGCGCCGGCCACGCAACCGGCACCA
CACCACAGAAGCTGTTTGTGGCCAATTATTCCCAGGACGTGAAGCAGTTCGCCAACG
GATTTGTGGTGCGGATCGGGGCCGCCGCCAACAGCACAGGCACCGTGATCATCTCTC
CCAGCACATCCGCCACCATCAGAAAGATCTACCCTGCCTTTATGCTGGGCAGCTCCGT
GGGCAACTTCTCCGATGGCAAGATGGGCAGGTTCTTTAATCACACACTGGTGCTGCTG
CCAGACGGATGCGGCACCCTGCTGAGGGCCTTCTACTGTATCCTGGAGCCCCGCTCTG
GAAATCACTGCCCTGCCGGCAACTCCTACACCTCTTTTGCCACATATCACACCCCTGC
CACAGACTGTTCCGATGGCAATTATAACCGGAATGCCAGCCTGAACTCCTTCAAGGA
GTACTTTAATCTGAGAAACTGCACCTTCATGTACACATATAATATCACCGAGGATGAG
ATCCTGGAGTGGTTCGGCATCACACAGACCGCCCAGGGCGTGCACCTGTTTTCTAGCA
GATACGTGGATCTGTATGGCGGCAACATGTTCCAGTTTGCCACACTGCCAGTGTATGA
CACCATCAAGTACTATAGCATCATCCCCCACTCTATCCGGAGCATCCAGTCCGACAGA
AAGGCCTGGGCCGCCTTCTACGTGTATAAGCTGCAGCCCCTGACCTTCCTGCTGGATT
TTTCCGTGGACGGCTACATCCGGAGAGCCATCGATTGCGGCTTTAACGACCTGTCTCA
GCTCCACTGTTCTTATGAGAGCTTCGATGTGGAGTCTGGCGTGTACAGCGTGTCCTCT
TTTGAGGCCAAGCCATCTGGCAGCGTGGTGGAGCAGGCAGAGGGAGTGGAGTGCGA
CTTCTCCCCACTGCTGTCTGGCACACCACCTCAGGTGTATAATTTCAAGAGGCTGGTG
TTTACAAACTGTAATTACAACCTGACCAAGCTGCTGTCCCTGTTCTCTGTGAACGACT
TTACCTGCAGCCAGATCTCCCCTGCCGCCATCGCCTCCAATTGTTATAGCTCCCTGAT
CCTGGATTACTTCTCTTATCCCCTGTCTATGAAGAGCGACCTGTCCGTGTCTAGCGCC
GGCCCTATCAGCCAGTTTAATTACAAGCAGTCCTTCTCTAACCCCACATGCCTGATCC
TGGCCACCGTGCCTCACAACCTGACCACAATCACAAAGCCACTGAAGTACTCCTATA
TCAATAAGTGCAGCAGGCTGCTGTCCGACGATCGCACCGAGGTGCCTCAGCTCGTGA
ACGCCAACCAGTACTCTCCATGCGTGAGCATCGTGCCATCCACCGTGTGGGAGGACG
GCGATTACTATAGAAAGCAGCTCAGCCCACTGGAGGGAGGAGGATGGCTGGTGGCC
AGCGGCTCCACAGTGGCCATGACCGAGCAGCTCCAGATGGGCTTCGGCATCACAGTG
CAGTACGGCACAGATACCAATAGCGTGTGCCCCAAGCTGGAGTTTGCCAACGACACC
AAGATCGCCTCCCAGCTCGGCAATTGCGTGGAGTACTCCCTGTATGGCGTGTCTGGCA
GAGGCGTGTTCCAGAACTGTACAGCCGTGGGCGTGCGGCAGCAGCGGTTCGTGTACG
ATGCCTATCAGAACCTGGTGGGCTACTATAGCGACGATGGCAATTACTATTGCCTGA
GGGCATGCGTGAGCGTGCCCGTGAGCGTGATCTACGACAAGGAGACAAAGACCCAC
GCCACCCTGTTCGGCTCCGTGGCCTGCGAGCACATCTCCTCTACAATGTCTCAGTATT
CTAGGAGCACCCGCTCTATGCTGAAGAGGCGCGACAGCACATACGGACCACTGCAGA
CCCCTGTGGGATGCGTGCTGGGCCTGGTGAACAGCAGCCTGTTTGTGGAGGATTGCA
AGCTGCCACTGGGCCAGTCTCTGTGCGCACTGCCAGACACCCCCAGCACACTGACCC
CACGGTCTGTGAGAAGCGTGCCCGGAGAGATGAGACTGGCCAGCATCGCCTTCAATC
ACCCTATCCAGGTGGATCAGCTCAACAGCAGCTACTTTAAGCTGAGCATCCCAACAA
ACTTCTCCTTTGGCGTGACCCAGGAGTATATCCAGACCACAATCCAGAAGGTGACCG
TGGACTGCAAGCAGTACGTGTGCAATGGCTTCCAGAAGTGCGAGCAGCTCCTGAGGG
AGTATGGCCAGTTTTGTTCCAAGATCAATCAGGCCCTGCACGGAGCCAACCTGAGGC
AGGACGATTCCGTGAGAAACCTGTTCGCCTCTGTGAAGTCCTCTCAGAGCTCCCCTAT
CATCCCAGGCTTCGGCGGCGACTTCAACCTGACCCTGCTGGAGCCCGTGTCCATCTCT
ACCGGCAGCAGGTCCGCCCGCAGCGCCATCGAGGATCTGCTGTTTGACAAGGTGACC
ATCGCCGACCCAGGCTACATGCAGGGCTATGACGATTGCATGCAGCAGGGACCAGCC
TCCGCCCGCGATCTGATCTGTGCCCAGTACGTGGCCGGCTATAAGGTGCTGCCACCCC
TGATGGACGTGAACATGGAGGCCGCCTATACATCTAGCCTGCTGGGCAGCATCGCAG
GAGTGGGATGGACCGCCGGCCTGTCCTCTTTCGCCGCAATCCCTTTTGCCCAGTCTAT
CTTCTACCGGCTGAACGGCGTGGGCATCACACAGCAGGTGCTGAGCGAGAATCAGAA
GCTGATCGCCAATAAGTTCAACCAGGCCCTGGGGGCCATGCAGACCGGCTTTACCAC
AACCAACGAGGCCTTCCAGAAGGTGCAGGATGCCGTGAACAATAACGCACAGGCCCT
GTCCAAGCTGGCCTCCGAGCTGTCTAATACCTTCGGGGCCATCAGCGCCAGCATCGG
CGACATCATCCAGCGCCTGGACGTGCTGGAGCAGGATGCCCAGATCGACAGGCTGAT
CAATGGCCGCCTGACAACCCTGAACGCCTTTGTGGCACAGCAGCTCGTGCGGAGCGA
GTCTGCCGCCCTGAGCGCCCAGCTCGCCAAGGACAAGGTGAACGAGTGCGTGAAGGC
CCAGAGCAAGCGGTCCGGCTTTTGTGGCCAGGGCACCCACATCGTGTCCTTCGTGGTG
AATGCCCCTAACGGCCTGTACTTTATGCATGTGGGCTACTATCCAAGCAACCACATCG
AGGTGGTGTCCGCCTATGGCCTGTGCGATGCCGCCAATCCTACAAACTGTATCGCCCC
AGTGAATGGCTACTTCATCAAGACCAATAACACACGGATCGTGGACGAGTGGTCCTA
CACCGGCAGCTCCTTTTATGCCCCCGAGCCTATCACATCTCTGAACACCAAGTACGTG
GCCCCACAGGTGACATATCAGAATATCAGCACCAACCTGCCTCCACCCCTGCTGGGC
AATTCCACCGGCATCGACTTCCAGGATGAGCTGGACGAGTTCTTTAAGAATGTGAGC
ACATCCATCCCCAACTTTGGCAGCCTGACCCAGATCAACACAACCCTGCTGGATCTGA
CATACGAGATGCTGTCTCTGCAGCAGGTGGTGAAGGCCCTGAATGAGAGCTACATCG
ACCTGAAGGAGCTGGGCAATTATACCTACTATAACAAGTGGCCTTGGTACATCTGGC
TGGGCTTCATCGCAGGCCTGGTGGCCCTGGCCCTGTGCGTGTTCTTTATCCTGTGCTGT
ACAGGCTGCGGCACCAATTGTATGGGCAAGCTGAAGTGTAACCGGTGCTGTGATAGA
TACGAGGAGTATGACCTGGAGCCACACAAGGTGCATGTGCACTGA
SEQ ID NO: 13 is a SARS-CoV-2 “Proline Modified” Spike Glycoprotein,
Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPRRARSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDPPEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKLHYT
SEQ ID NO: 14 is a SARS-CoV-2 “Proline Modified” Spike Glycoprotein,
Nucleotide Sequence
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGTC
TAACATAATAAGAGGCTGGATTTTTGGTACTACTTTAGATTCGAAGACCCAGTCCCTA
CTTATTGTTAATAACGCTACTAATGTTGTTATTAAAGTCTGTGAATTTCAATTTTGTAA
TGATCCATTTTTGGGTGTTTATTACCACAAAAACAACAAAAGTTGGATGGAAAGTGA
GTTCAGAGTTTATTCTAGTGCGAATAATTGCACTTTTGAATATGTCTCTCAGCCTTTTC
TTATGGACCTTGAAGGAAAACAGGGTAATTTCAAAAATCTTAGGGAATTTGTGTTTA
AGAATATTGATGGTTATTTTAAAATATATTCTAAGCACACGCCTATTAATTTAGTGCG
TGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTG
ATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGT
GCACTTGACCCTCTCTCAGAAACAAAGTGTACGTTGAAATCCTTCACTGTAGAAAAA
GGAATCTATCAAACTTCTAACTTTAGAGTCCAACCAACAGAATCTATTGTTAGATTTC
CTAATATTACAAACTTGTGCCCTTTTGGTGAAGTTTTTAACGCCACCAGATTTGCATCT
GTTTATGCTTGGAACAGGAAGAGAATCAGCAACTGTGTTGCTGATTATTCTGTCCTAT
ATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTACCAG
ATGATTTTACAGGCTGCGTTATAGCTTGGAATTCTAACAATCTTGATTCTAAGGTTGG
TGGTAATTATAATTACCTGTATAGATTGTTTAGGAAGTCTAATCTCAAACCTTTTGAG
AGAGATATTTCAACTGAAATCTATCAGGCCGGTAGCACACCTTGTAATGGTGTTGAA
GGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACT
GTTTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCCTTT
CCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAG
TCCCTGTTGCTATTCATGCAGATCAACTTACTCCTACTTGGCGTGTTTATTCTACAGGT
TCTAATGTTTTTCAAACACGTGCAGGCTGTTTAATAGGGGCTGAACATGTCAACAACT
CATATGAGTGTGACATACCCATTGGTGCAGGTATATGCGCTAGTTATCAGACTCAGAC
TAATTCTCCTCGGCGGGCACGTAGTGTAGCTAGTCAATCCATCATTGCCTACACTATG
TCACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCAGT
AGATTGTACAATGTACATTTGTGGTGATTCAACTGAATGCAGCAATCTTTTGTTGCAA
TATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAG
CAAGAGGTCATTTATTGAAGATCTACTTTTCAACAAAGTGACACTTGCAGATGCTGGC
TTCATCAAACAATATGGTGATTGCCTTGGTGATATTGCTGCTAGAGACCTCATTTGTG
CACAAAAGTTTAACGGCCTTACTGTTTTGCCACCTTTGCTCACAGATGAAATGATTGC
TCAATACACTTCTGCACTGTTAGCGGGTACAATCACTTCTGGTTGGACCTTTGGTGCA
GGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAAACT
TCAAGATGTGGTCAACCAAAATGCACAAGCTTTAAACACGCTTGTTAAACAACTTAG
CTCCAATTTTGGTGCAATTTCAAGTGTTTTAAATGATATCCTTTCACGTCTTGACCCTC
CTGAGGCTGAAGTGCAAATTGATAGGTTGATCACAGGCAGACTTCAAAGTTTGCAGA
CATATGTGACTCAACAATTAATTAGAGCTGCAGAAATCAGAGCTTCTGCTAATCTTGC
TGCTACTAAAATGTCAGAGTGTGTACTTGGACAATCAAAAAGAGTTGATTTTTGTGGA
AAGGGCTATCATCTTATGTCCTTCCCTCAGTCAGCACCTCATGGTGTAGTCTTCTTGCA
TGTGACTTATGTCCCTGCACAAGAAAAGAACTTCACAACTGCTCCTGCCATTTGTCAT
GATGGAAAAGCACACTTTCCTCGTGAAGGTGTCTTTGTTTCAAATGGCACACACTGGT
TTGTAACACAAAGGAATTTTTATGAACCACAAATCATTACTACAGACAACACATTTGT
GTCTGGTAACTGTGATGTTGTAATAGGAATTGTCAACAACACAGTTTATGATCCTTTG
CAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTC
AAAAAGAAATTGACCGCCTCAATGAGGTTGCCAAGAATTTAAATGAATCTCTCATCG
ATCTCCAAGAACTTGGAAAGTATGAGCAGTATATAAAATGGCCATGGTACATTTGGC
TAGGTTTTATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTGCAAATTT
GATGAAGACGACTCTGAGCCAGTGCTCAAAGGAGTCAAATTACATTACACATAA
SEQ ID NO: 15 is a SARS-CoV-2 “Proline Modified” Spike Glycoprotein,
Nucleotide Sequence, Codon Optimized for Expression In Human Cells
ATGTTCGTGTTTCTGGTGCTGCTGCCTCTGGTGAGCTCCCAGTGCGTGAACCTGACCA
CAAGGACCCAGCTCCCCCCTGCCTATACCAATTCCTTCACACGGGGCGTGTACTATCC
AGACAAGGTGTTTAGATCTAGCGTGCTGCACTCCACACAGGATCTGTTTCTGCCCTTC
TTTTCTAACGTGACCTGGTTCCACGCCATCCATGTGAGCGGCACCAATGGCACAAAGC
GGTTCGACAATCCTGTGCTGCCCTTCAACGATGGCGTGTACTTCGCCTCCACCGAGAA
GTCTAACATCATCAGAGGCTGGATCTTTGGCACCACACTGGACAGCAAGACACAGTC
CCTGCTGATCGTGAACAATGCCACCAACGTGGTCATCAAGGTGTGCGAGTTCCAGTTT
TGTAATGATCCTTTCCTGGGCGTGTACTATCACAAGAACAATAAGTCTTGGATGGAGA
GCGAGTTTCGCGTGTATTCCTCTGCCAACAATTGCACATTTGAGTACGTGTCCCAGCC
ATTCCTGATGGACCTGGAGGGCAAGCAGGGCAATTTCAAGAACCTGAGGGAGTTCGT
GTTTAAGAATATCGATGGCTACTTCAAGATCTACTCCAAGCACACCCCTATCAACCTG
GTGCGCGACCTGCCACAGGGCTTCTCTGCCCTGGAGCCTCTGGTGGATCTGCCAATCG
GCATCAACATCACCCGGTTTCAGACACTGCTGGCCCTGCACAGAAGCTACCTGACAC
CTGGCGACAGCTCCTCTGGATGGACCGCCGGGGCCGCCGCCTACTATGTGGGCTATCT
GCAGCCAAGGACCTTCCTGCTGAAGTACAACGAGAATGGCACCATCACAGACGCAGT
GGATTGCGCCCTGGACCCCCTGTCTGAGACCAAGTGTACACTGAAGAGCTTTACCGT
GGAGAAGGGCATCTATCAGACAAGCAATTTCAGGGTGCAGCCCACCGAGTCCATCGT
GCGCTTTCCAAATATCACAAACCTGTGCCCCTTTGGCGAGGTGTTCAACGCAACCAGG
TTCGCCAGCGTGTACGCATGGAATAGGAAGCGCATCTCCAACTGCGTGGCCGACTAT
TCTGTGCTGTACAACAGCGCCTCCTTCTCTACCTTTAAGTGCTATGGCGTGAGCCCCA
CAAAGCTGAATGACCTGTGCTTTACCAACGTGTACGCCGATTCCTTCGTGATCAGGGG
CGACGAGGTGCGCCAGATCGCACCAGGACAGACAGGCAAGATCGCCGACTACAATT
ATAAGCTGCCCGACGATTTCACCGGCTGCGTGATCGCCTGGAACTCTAACAATCTGG
ATAGCAAAGTGGGCGGCAACTACAATTATCTGTACCGGCTGTTTAGAAAGTCTAATC
TGAAGCCTTTCGAGAGGGACATCTCCACAGAGATCTACCAGGCCGGCTCTACCCCAT
GCAATGGCGTGGAGGGCTTTAACTGTTATTTCCCCCTGCAGAGCTACGGCTTCCAGCC
TACAAACGGCGTGGGCTATCAGCCATACCGCGTGGTGGTGCTGTCTTTTGAGCTGCTG
CACGCACCAGCAACAGTGTGCGGACCTAAGAAGAGCACCAATCTGGTGAAGAACAA
GTGCGTGAACTTCAACTTCAACGGCCTGACCGGCACAGGCGTGCTGACCGAGTCCAA
CAAGAAGTTCCTGCCCTTTCAGCAGTTCGGCAGGGACATCGCAGATACCACAGACGC
CGTGCGCGACCCCCAGACCCTGGAGATCCTGGACATCACACCTTGCTCCTTCGGCGGC
GTGTCTGTGATCACACCTGGCACCAATACAAGCAACCAGGTGGCCGTGCTGTATCAG
GACGTGAATTGTACCGAGGTGCCAGTGGCCATCCACGCCGATCAGCTCACCCCCACA
TGGCGGGTGTACTCTACCGGCAGCAACGTGTTCCAGACAAGAGCCGGCTGCCTGATC
GGAGCCGAGCATGTGAACAATAGCTATGAGTGCGACATCCCCATCGGAGCCGGCATC
TGTGCCTCCTACCAGACCCAGACAAACTCCCCTCGGAGAGCCCGGTCTGTGGCCAGC
CAGTCCATCATCGCCTATACCATGAGCCTGGGGGCCGAGAACAGCGTGGCCTACTCC
AACAATTCTATCGCCATCCCCACCAACTTCACAATCTCCGTGACCACAGAGATCCTGC
CTGTGAGCATGACCAAGACATCCGTGGACTGCACAATGTATATCTGTGGCGATTCCA
CCGAGTGCTCTAACCTGCTGCTGCAGTACGGCTCTTTTTGTACCCAGCTCAACAGAGC
CCTGACAGGCATCGCCGTGGAGCAGGACAAGAACACACAGGAGGTGTTCGCCCAGG
TGAAGCAGATCTACAAGACCCCACCCATCAAGGACTTTGGCGGCTTCAACTTCAGCC
AGATCCTGCCAGATCCCAGCAAGCCTTCCAAGCGGTCTTTTATCGAGGACCTGCTGTT
CAACAAGGTGACCCTGGCCGATGCCGGCTTCATCAAGCAGTATGGCGATTGCCTGGG
CGACATCGCCGCCAGAGACCTGATCTGTGCCCAGAAGTTTAATGGCCTGACCGTGCT
GCCTCCACTGCTGACAGATGAGATGATCGCCCAGTACACATCTGCCCTGCTGGCCGG
CACCATCACAAGCGGATGGACCTTCGGGGCCGGGGCCGCCCTGCAGATCCCATTTGC
CATGCAGATGGCCTATCGGTTCAACGGCATCGGCGTGACCCAGAATGTGCTGTACGA
GAACCAGAAGCTGATCGCCAATCAGTTTAACTCCGCCATCGGCAAGATCCAGGACTC
TCTGAGCTCCACAGCCAGCGCCCTGGGCAAGCTGCAGGATGTGGTGAATCAGAACGC
CCAGGCCCTGAATACCCTGGTGAAGCAGCTCAGCAGCAACTTCGGGGCCATCAGCAG
CGTGCTGAACGACATCCTGAGCCGGCTGGACCCCCCTGAGGCAGAGGTGCAGATCGA
CCGGCTGATCACAGGCAGACTGCAGTCCCTGCAGACCTACGTGACACAGCAGCTCAT
CAGGGCCGCCGAGATCAGGGCCTCTGCCAATCTGGCCGCCACCAAGATGAGCGAGTG
CGTGCTGGGCCAGTCCAAGAGAGTGGACTTTTGTGGCAAGGGCTATCACCTGATGAG
CTTCCCACAGTCCGCCCCCCACGGAGTGGTGTTTCTGCATGTGACCTACGTGCCTGCC
CAGGAGAAGAACTTCACCACAGCCCCAGCCATCTGCCACGATGGCAAGGCCCACTTT
CCCAGGGAGGGCGTGTTCGTGAGCAACGGCACCCACTGGTTTGTGACACAGCGCAAT
TTCTACGAGCCTCAGATCATCACCACAGACAATACATTCGTGTCCGGCAACTGTGACG
TGGTCATCGGCATCGTGAACAATACCGTGTATGATCCCCTGCAGCCTGAGCTGGACTC
TTTTAAGGAGGAGCTGGATAAGTACTTCAAGAATCACACCAGCCCCGACGTGGATCT
GGGCGACATCTCTGGCATCAATGCCAGCGTGGTGAACATCCAGAAGGAGATCGACAG
GCTGAACGAGGTGGCCAAGAATCTGAACGAGTCCCTGATCGATCTGCAGGAGCTGGG
CAAGTATGAGCAGTACATCAAGTGGCCATGGTATATCTGGCTGGGCTTCATCGCCGG
CCTGATCGCCATCGTGATGGTGACCATCATGCTGTGCTGTATGACAAGCTGCTGTTCC
TGCCTGAAGGGCTGCTGTTCTTGTGGCTCCTGCTGTAAGTTTGATGAGGACGATAGCG
AGCCCGTGCTGAAGGGCGTGAAGCTGCACTACACCTGA
SEQ ID NO: 16 is a SARS-CoV-2 “Furin Cleavage Modified” Spike
Glycoprotein, Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPGSASSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAICHDGKAHFPR
EGVEVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKLHYT
SEQ ID NO: 17 is a SARS-CoV-2 “Furin Cleavage Modified” Spike
Glycoprotein, Nucleotide Sequence
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGTC
TAACATAATAAGAGGCTGGATTTTTGGTACTACTTTAGATTCGAAGACCCAGTCCCTA
CTTATTGTTAATAACGCTACTAATGTTGTTATTAAAGTCTGTGAATTTCAATTTTGTAA
TGATCCATTTTTGGGTGTTTATTACCACAAAAACAACAAAAGTTGGATGGAAAGTGA
GTTCAGAGTTTATTCTAGTGCGAATAATTGCACTTTTGAATATGTCTCTCAGCCTTTTC
TTATGGACCTTGAAGGAAAACAGGGTAATTTCAAAAATCTTAGGGAATTTGTGTTTA
AGAATATTGATGGTTATTTTAAAATATATTCTAAGCACACGCCTATTAATTTAGTGCG
TGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTG
ATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGT
GCACTTGACCCTCTCTCAGAAACAAAGTGTACGTTGAAATCCTTCACTGTAGAAAAA
GGAATCTATCAAACTTCTAACTTTAGAGTCCAACCAACAGAATCTATTGTTAGATTTC
CTAATATTACAAACTTGTGCCCTTTTGGTGAAGTTTTTAACGCCACCAGATTTGCATCT
GTTTATGCTTGGAACAGGAAGAGAATCAGCAACTGTGTTGCTGATTATTCTGTCCTAT
ATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTACCAG
ATGATTTTACAGGCTGCGTTATAGCTTGGAATTCTAACAATCTTGATTCTAAGGTTGG
TGGTAATTATAATTACCTGTATAGATTGTTTAGGAAGTCTAATCTCAAACCTTTTGAG
AGAGATATTTCAACTGAAATCTATCAGGCCGGTAGCACACCTTGTAATGGTGTTGAA
GGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACT
GTTTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCCTTT
CCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAG
TCCCTGTTGCTATTCATGCAGATCAACTTACTCCTACTTGGCGTGTTTATTCTACAGGT
TCTAATGTTTTTCAAACACGTGCAGGCTGTTTAATAGGGGCTGAACATGTCAACAACT
CATATGAGTGTGACATACCCATTGGTGCAGGTATATGCGCTAGTTATCAGACTCAGAC
TAATTCTCCTGGTAGTGCAAGTAGTGTAGCTAGTCAATCCATCATTGCCTACACTATG
TCACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCAGT
AGATTGTACAATGTACATTTGTGGTGATTCAACTGAATGCAGCAATCTTTTGTTGCAA
TATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAG
CAAGAGGTCATTTATTGAAGATCTACTTTTCAACAAAGTGACACTTGCAGATGCTGGC
TTCATCAAACAATATGGTGATTGCCTTGGTGATATTGCTGCTAGAGACCTCATTTGTG
CACAAAAGTTTAACGGCCTTACTGTTTTGCCACCTTTGCTCACAGATGAAATGATTGC
TCAATACACTTCTGCACTGTTAGCGGGTACAATCACTTCTGGTTGGACCTTTGGTGCA
GGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAAACT
TCAAGATGTGGTCAACCAAAATGCACAAGCTTTAAACACGCTTGTTAAACAACTTAG
CTCCAATTTTGGTGCAATTTCAAGTGTTTTAAATGATATCCTTTCACGTCTTGACAAAG
TTGAGGCTGAAGTGCAAATTGATAGGTTGATCACAGGCAGACTTCAAAGTTTGCAGA
CATATGTGACTCAACAATTAATTAGAGCTGCAGAAATCAGAGCTTCTGCTAATCTTGC
TGCTACTAAAATGTCAGAGTGTGTACTTGGACAATCAAAAAGAGTTGATTTTTGTGGA
AAGGGCTATCATCTTATGTCCTTCCCTCAGTCAGCACCTCATGGTGTAGTCTTCTTGCA
TGTGACTTATGTCCCTGCACAAGAAAAGAACTTCACAACTGCTCCTGCCATTTGTCAT
GATGGAAAAGCACACTTTCCTCGTGAAGGTGTCTTTGTTTCAAATGGCACACACTGGT
TTGTAACACAAAGGAATTTTTATGAACCACAAATCATTACTACAGACAACACATTTGT
GTCTGGTAACTGTGATGTTGTAATAGGAATTGTCAACAACACAGTTTATGATCCTTTG
CAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTC
AAAAAGAAATTGACCGCCTCAATGAGGTTGCCAAGAATTTAAATGAATCTCTCATCG
ATCTCCAAGAACTTGGAAAGTATGAGCAGTATATAAAATGGCCATGGTACATTTGGC
TAGGTTTTATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTGCAAATTT
GATGAAGACGACTCTGAGCCAGTGCTCAAAGGAGTCAAATTACATTACACATAA
SEQ ID NO: 18 is a SARS-CoV-2 “Furin Cleavage Modified” Spike
Glycoprotein, Nucleotide Sequence, Codon Optimized For Expression In
Human Cells
ATGTTCGTGTTTCTGGTGCTGCTGCCTCTGGTGAGCTCCCAGTGCGTGAACCTGACCA
CAAGGACCCAGCTCCCCCCTGCCTATACCAATTCCTTCACAAGGGGCGTGTACTATCC
CGACAAGGTGTTTCGCTCTAGCGTGCTGCACAGCACACAGGATCTGTTTCTGCCTTTC
TTTTCCAACGTGACCTGGTTCCACGCCATCCATGTGAGCGGCACCAATGGCACAAAG
AGGTTCGACAATCCAGTGCTGCCCTTTAACGATGGCGTGTACTTCGCCTCTACCGAGA
AGAGCAACATCATCCGCGGCTGGATCTTTGGCACCACACTGGACTCCAAGACACAGT
CTCTGCTGATCGTGAACAATGCCACCAACGTGGTCATCAAGGTGTGCGAGTTCCAGTT
TTGTAATGATCCATTCCTGGGCGTGTACTATCACAAGAACAATAAGAGCTGGATGGA
GTCCGAGTTTCGCGTGTATTCCTCTGCCAACAATTGCACATTTGAGTACGTGTCCCAG
CCCTTCCTGATGGACCTGGAGGGCAAGCAGGGCAATTTCAAGAACCTGCGGGAGTTC
GTGTTTAAGAATATCGATGGCTACTTCAAGATCTACAGCAAGCACACCCCAATCAAC
CTGGTGAGAGACCTGCCACAGGGCTTCTCCGCCCTGGAGCCACTGGTGGATCTGCCC
ATCGGCATCAACATCACCAGGTTTCAGACACTGCTGGCCCTGCACCGCAGCTACCTG
ACACCAGGCGACAGCTCCTCTGGATGGACCGCCGGGGCCGCCGCCTACTATGTGGGC
TATCTGCAGCCCCGGACCTTCCTGCTGAAGTACAACGAGAATGGCACCATCACAGAC
GCAGTGGATTGCGCCCTGGACCCCCTGTCCGAGACCAAGTGTACACTGAAGTCTTTTA
CCGTGGAGAAGGGCATCTATCAGACATCTAATTTCCGGGTGCAGCCTACCGAGAGCA
TCGTGAGATTTCCCAATATCACAAACCTGTGCCCTTTTGGCGAGGTGTTCAACGCCAC
CAGATTCGCCAGCGTGTACGCCTGGAATCGGAAGAGAATCAGCAACTGCGTGGCCGA
CTATTCCGTGCTGTACAACTCTGCCAGCTTCTCCACCTTTAAGTGCTATGGCGTGTCTC
CCACAAAGCTGAATGACCTGTGCTTTACCAACGTGTACGCCGATAGCTTCGTGATCAG
GGGCGACGAGGTGAGACAGATCGCACCAGGACAGACAGGCAAGATCGCAGACTACA
ATTATAAGCTGCCTGACGATTTCACCGGCTGCGTGATCGCCTGGAACAGCAACAATCT
GGATTCCAAAGTGGGCGGCAACTACAATTATCTGTACAGGCTGTTTCGCAAGTCCAA
TCTGAAGCCATTCGAGCGGGACATCAGCACAGAGATCTACCAGGCAGGCTCCACCCC
ATGCAATGGAGTGGAGGGCTTTAACTGTTATTTCCCTCTGCAGTCTTACGGCTTCCAG
CCAACAAACGGCGTGGGCTATCAGCCCTACAGAGTGGTGGTGCTGTCCTTTGAGCTG
CTGCACGCACCTGCAACAGTGTGCGGACCAAAGAAGTCTACCAATCTGGTGAAGAAC
AAGTGCGTGAACTTCAACTTCAACGGCCTGACCGGCACAGGCGTGCTGACCGAGTCC
AACAAGAAGTTCCTGCCTTTTCAGCAGTTCGGCAGAGACATCGCCGATACCACAGAC
GCCGTGAGAGACCCTCAGACCCTGGAGATCCTGGACATCACACCATGCTCTTTCGGC
GGCGTGAGCGTGATCACACCAGGCACCAATACAAGCAACCAGGTGGCCGTGCTGTAT
CAGGACGTGAATTGTACCGAGGTGCCCGTGGCCATCCACGCAGATCAGCTCACCCCT
ACATGGAGGGTGTACTCCACCGGCTCTAACGTGTTCCAGACACGCGCCGGATGCCTG
ATCGGAGCCGAGCATGTGAACAATTCTTATGAGTGCGACATCCCTATCGGAGCCGGC
ATCTGTGCCAGCTACCAGACCCAGACAAACAGCCCAGGCTCCGCCAGCTCCGTGGCC
TCTCAGAGCATCATCGCCTATACCATGAGCCTGGGGGCCGAGAATAGCGTGGCCTAC
TCTAACAATAGCATCGCCATCCCTACCAACTTCACAATCTCCGTGACCACAGAGATCC
TGCCAGTGTCCATGACCAAGACATCTGTGGACTGCACAATGTATATCTGTGGCGATTC
TACCGAGTGCAGCAACCTGCTGCTGCAGTACGGCAGCTTTTGTACCCAGCTCAACCG
GGCCCTGACAGGAATCGCAGTGGAGCAGGACAAGAACACACAGGAGGTGTTCGCCC
AGGTGAAGCAGATCTACAAGACCCCACCCATCAAGGACTTTGGCGGCTTCAACTTCA
GCCAGATCCTGCCCGATCCTTCCAAGCCATCTAAGAGGAGCTTTATCGAGGACCTGCT
GTTCAACAAGGTGACCCTGGCCGATGCCGGCTTCATCAAGCAGTATGGCGATTGCCT
GGGCGACATCGCAGCCCGCGACCTGATCTGTGCCCAGAAGTTTAATGGCCTGACCGT
GCTGCCTCCACTGCTGACAGATGAGATGATCGCACAGTACACATCCGCCCTGCTGGC
CGGCACCATCACATCTGGATGGACCTTCGGGGCCGGGGCCGCCCTGCAGATCCCCTTT
GCCATGCAGATGGCCTATAGATTCAACGGCATCGGCGTGACCCAGAATGTGCTGTAC
GAGAACCAGAAGCTGATCGCCAATCAGTTTAACTCCGCCATCGGCAAGATCCAGGAC
TCCCTGTCTAGCACAGCCTCTGCCCTGGGCAAGCTGCAGGATGTGGTGAATCAGAAC
GCCCAGGCCCTGAATACCCTGGTGAAGCAGCTCAGCAGCAACTTCGGGGCCATCAGC
AGCGTGCTGAACGACATCCTGAGCCGGCTGGACAAGGTGGAGGCAGAGGTGCAGAT
CGACAGGCTGATCACAGGCCGCCTGCAGAGCCTGCAGACCTACGTGACACAGCAGCT
CATCAGGGCCGCCGAGATCAGAGCCTCCGCCAATCTGGCCGCCACCAAGATGTCTGA
GTGCGTGCTGGGCCAGAGCAAGCGCGTGGACTTTTGTGGCAAGGGCTATCACCTGAT
GTCCTTCCCACAGTCTGCCCCTCACGGAGTGGTGTTTCTGCATGTGACCTACGTGCCA
GCCCAGGAGAAGAACTTCACCACAGCCCCCGCAATCTGCCACGATGGCAAGGCACAC
TTTCCTCGGGAGGGCGTGTTCGTGTCTAACGGCACCCACTGGTTTGTGACACAGAGAA
ATTTCTACGAGCCACAGATCATCACCACAGACAATACATTCGTGAGCGGCAACTGTG
ACGTGGTCATCGGCATCGTGAACAATACCGTGTATGATCCTCTGCAGCCAGAGCTGG
ACTCCTTTAAGGAGGAGCTGGATAAGTACTTCAAGAATCACACCTCTCCCGACGTGG
ATCTGGGCGACATCAGCGGCATCAATGCCTCCGTGGTGAACATCCAGAAGGAGATCG
ACAGGCTGAACGAGGTGGCCAAGAATCTGAACGAGTCCCTGATCGATCTGCAGGAGC
TGGGCAAGTATGAGCAGTACATCAAGTGGCCCTGGTATATCTGGCTGGGCTTCATCG
CCGGCCTGATCGCCATCGTGATGGTGACCATCATGCTGTGCTGTATGACATCCTGCTG
TTCTTGCCTGAAGGGCTGCTGTAGCTGTGGCTCCTGCTGTAAGTTTGATGAGGACGAT
AGCGAGCCTGTGCTGAAGGGCGTGAAGCTGCACTACACCTGA
SEQ ID NO: 19 is a SARS-CoV-2 “Proline & Furin Cleavage Modified” Spike
Glycoprotein, Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPGSASSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDPPEAEVQIIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKWP
WYIWLGFIAGLIAIVMVTIMLCCMTSCCSCLKGCCSCGSCCKFDEDDSEPVLKGVKLHYT
SEQ ID NO: 20 is a SARS-CoV-2 “Proline & Furin Cleavage Modified” Spike
Glycoprotein, Nucleotide Sequence
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGTC
TAACATAATAAGAGGCTGGATTTTTGGTACTACTTTAGATTCGAAGACCCAGTCCCTA
CTTATTGTTAATAACGCTACTAATGTTGTTATTAAAGTCTGTGAATTTCAATTTTGTAA
TGATCCATTTTTGGGTGTTTATTACCACAAAAACAACAAAAGTTGGATGGAAAGTGA
GTTCAGAGTTTATTCTAGTGCGAATAATTGCACTTTTGAATATGTCTCTCAGCCTTTTC
TTATGGACCTTGAAGGAAAACAGGGTAATTTCAAAAATCTTAGGGAATTTGTGTTTA
AGAATATTGATGGTTATTTTAAAATATATTCTAAGCACACGCCTATTAATTTAGTGCG
TGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTG
ATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGT
GCACTTGACCCTCTCTCAGAAACAAAGTGTACGTTGAAATCCTTCACTGTAGAAAAA
GGAATCTATCAAACTTCTAACTTTAGAGTCCAACCAACAGAATCTATTGTTAGATTTC
CTAATATTACAAACTTGTGCCCTTTTGGTGAAGTTTTTAACGCCACCAGATTTGCATCT
GTTTATGCTTGGAACAGGAAGAGAATCAGCAACTGTGTTGCTGATTATTCTGTCCTAT
ATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTACCAG
ATGATTTTACAGGCTGCGTTATAGCTTGGAATTCTAACAATCTTGATTCTAAGGTTGG
TGGTAATTATAATTACCTGTATAGATTGTTTAGGAAGTCTAATCTCAAACCTTTTGAG
AGAGATATTTCAACTGAAATCTATCAGGCCGGTAGCACACCTTGTAATGGTGTTGAA
GGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACT
GTTTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCCTTT
CCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAG
TCCCTGTTGCTATTCATGCAGATCAACTTACTCCTACTTGGCGTGTTTATTCTACAGGT
TCTAATGTTTTTCAAACACGTGCAGGCTGTTTAATAGGGGCTGAACATGTCAACAACT
CATATGAGTGTGACATACCCATTGGTGCAGGTATATGCGCTAGTTATCAGACTCAGAC
TAATTCTCCTGGTAGTGCAAGTAGTGTAGCTAGTCAATCCATCATTGCCTACACTATG
TCACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCAGT
AGATTGTACAATGTACATTTGTGGTGATTCAACTGAATGCAGCAATCTTTTGTTGCAA
TATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAG
CAAGAGGTCATTTATTGAAGATCTACTTTTCAACAAAGTGACACTTGCAGATGCTGGC
TTCATCAAACAATATGGTGATTGCCTTGGTGATATTGCTGCTAGAGACCTCATTTGTG
CACAAAAGTTTAACGGCCTTACTGTTTTGCCACCTTTGCTCACAGATGAAATGATTGC
TCAATACACTTCTGCACTGTTAGCGGGTACAATCACTTCTGGTTGGACCTTTGGTGCA
GGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAAACT
TCAAGATGTGGTCAACCAAAATGCACAAGCTTTAAACACGCTTGTTAAACAACTTAG
CTCCAATTTTGGTGCAATTTCAAGTGTTTTAAATGATATCCTTTCACGTCTTGACCCTC
CTGAGGCTGAAGTGCAAATTGATAGGTTGATCACAGGCAGACTTCAAAGTTTGCAGA
CATATGTGACTCAACAATTAATTAGAGCTGCAGAAATCAGAGCTTCTGCTAATCTTGC
TGCTACTAAAATGTCAGAGTGTGTACTTGGACAATCAAAAAGAGTTGATTTTTGTGGA
AAGGGCTATCATCTTATGTCCTTCCCTCAGTCAGCACCTCATGGTGTAGTCTTCTTGCA
TGTGACTTATGTCCCTGCACAAGAAAAGAACTTCACAACTGCTCCTGCCATTTGTCAT
GATGGAAAAGCACACTTTCCTCGTGAAGGTGTCTTTGTTTCAAATGGCACACACTGGT
TTGTAACACAAAGGAATTTTTATGAACCACAAATCATTACTACAGACAACACATTTGT
GTCTGGTAACTGTGATGTTGTAATAGGAATTGTCAACAACACAGTTTATGATCCTTTG
CAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTC
AAAAAGAAATTGACCGCCTCAATGAGGTTGCCAAGAATTTAAATGAATCTCTCATCG
ATCTCCAAGAACTTGGAAAGTATGAGCAGTATATAAAATGGCCATGGTACATTTGGC
TAGGTTTTATAGCTGGCTTGATTGCCATAGTAATGGTGACAATTATGCTTTGCTGTAT
GACCAGTTGCTGTAGTTGTCTCAAGGGCTGTTGTTCTTGTGGATCCTGCTGCAAATTT
GATGAAGACGACTCTGAGCCAGTGCTCAAAGGAGTCAAATTACATTACACATAA
SEQ ID NO: 21 is a SARS-Cov-2 “Proline & Furin Cleavage Modified” Spike
Glycoprotein, Nucleotide Sequence, Codon Optimized for Expression in
Human Cells
ATGTTCGTGTTTCTGGTGCTGCTGCCTCTGGTGAGCTCCCAGTGCGTGAACCTGACCA
CAAGGACCCAGCTCCCCCCTGCCTATACCAATTCCTTCACAAGGGGCGTGTACTATCC
AGACAAGGTGTTTCGCTCTAGCGTGCTGCACAGCACACAGGATCTGTTTCTGCCCTTC
TTTTCCAACGTGACCTGGTTCCACGCCATCCATGTGAGCGGCACCAATGGCACAAAG
AGGTTCGACAATCCTGTGCTGCCCTTCAACGATGGCGTGTACTTCGCCTCTACCGAGA
AGAGCAACATCATCCGCGGCTGGATCTTTGGCACCACACTGGACTCCAAGACACAGT
CTCTGCTGATCGTGAACAATGCCACCAACGTGGTCATCAAGGTGTGCGAGTTCCAGTT
TTGTAATGATCCTTTCCTGGGCGTGTACTATCACAAGAACAATAAGAGCTGGATGGA
GTCCGAGTTTCGCGTGTATTCCTCTGCCAACAATTGCACATTTGAGTACGTGTCCCAG
CCATTCCTGATGGACCTGGAGGGCAAGCAGGGCAATTTCAAGAACCTGCGGGAGTTC
GTGTTTAAGAATATCGATGGCTACTTCAAGATCTACAGCAAGCACACCCCTATCAACC
TGGTGAGAGACCTGCCACAGGGCTTCTCCGCCCTGGAGCCTCTGGTGGATCTGCCAAT
CGGCATCAACATCACCAGGTTTCAGACACTGCTGGCCCTGCACCGCAGCTACCTGAC
ACCTGGCGACAGCTCCTCTGGATGGACCGCCGGGGCCGCCGCCTACTATGTGGGCTA
TCTGCAGCCACGGACCTTCCTGCTGAAGTACAACGAGAATGGCACCATCACAGACGC
AGTGGATTGCGCCCTGGACCCCCTGTCCGAGACCAAGTGTACACTGAAGTCTTTTACC
GTGGAGAAGGGCATCTATCAGACATCTAATTTCCGGGTGCAGCCCACCGAGAGCATC
GTGAGATTTCCAAATATCACAAACCTGTGCCCCTTTGGCGAGGTGTTCAACGCCACCA
GATTCGCCAGCGTGTACGCCTGGAATCGGAAGAGAATCAGCAACTGCGTGGCCGACT
ATTCCGTGCTGTACAACTCTGCCAGCTTCTCCACCTTTAAGTGCTATGGCGTGTCTCCC
ACAAAGCTGAATGACCTGTGCTTTACCAACGTGTACGCCGATAGCTTCGTGATCAGG
GGCGACGAGGTGAGACAGATCGCACCAGGCCAGACAGGCAAGATCGCCGACTACAA
TTATAAGCTGCCCGACGATTTCACCGGCTGCGTGATCGCCTGGAACAGCAACAATCT
GGATTCCAAAGTGGGCGGCAACTACAATTATCTGTACAGGCTGTTTCGCAAGTCCAA
TCTGAAGCCTTTCGAGCGGGACATCAGCACAGAGATCTACCAGGCCGGCTCCACCCC
ATGCAATGGCGTGGAGGGCTTTAACTGTTATTTCCCCCTGCAGTCTTACGGCTTCCAG
CCTACAAACGGCGTGGGCTATCAGCCATACAGAGTGGTGGTGCTGTCCTTTGAGCTG
CTGCACGCACCAGCAACAGTGTGCGGACCTAAGAAGTCTACCAATCTGGTGAAGAAC
AAGTGCGTGAACTTCAACTTCAACGGCCTGACCGGCACAGGCGTGCTGACCGAGTCC
AACAAGAAGTTCCTGCCCTTTCAGCAGTTCGGCAGAGACATCGCCGATACCACAGAC
GCCGTGAGAGACCCCCAGACCCTGGAGATCCTGGACATCACACCTTGCTCTTTCGGC
GGCGTGAGCGTGATCACACCTGGCACCAATACAAGCAACCAGGTGGCCGTGCTGTAT
CAGGACGTGAATTGTACCGAGGTGCCAGTGGCCATCCACGCCGATCAGCTCACCCCC
ACATGGAGGGTGTACTCCACCGGCTCTAACGTGTTCCAGACACGCGCCGGATGCCTG
ATCGGAGCCGAGCATGTGAACAATTCTTATGAGTGCGACATCCCCATCGGAGCCGGC
ATCTGTGCCAGCTACCAGACCCAGACAAACAGCCCTGGCTCCGCCAGCTCCGTGGCC
TCTCAGAGCATCATCGCCTATACCATGAGCCTGGGGGCCGAGAATAGCGTGGCCTAC
TCTAACAATAGCATCGCCATCCCCACCAACTTCACAATCTCCGTGACCACAGAGATCC
TGCCCGTGAGCATGACCAAGACATCTGTGGACTGCACAATGTATATCTGTGGCGATTC
TACCGAGTGCAGCAACCTGCTGCTGCAGTACGGCAGCTTTTGTACCCAGCTCAACCG
GGCCCTGACAGGAATCGCAGTGGAGCAGGACAAGAACACACAGGAGGTGTTCGCCC
AGGTGAAGCAGATCTACAAGACCCCACCCATCAAGGACTTTGGCGGCTTCAACTTCA
GCCAGATCCTGCCAGATCCCTCCAAGCCTTCTAAGAGGAGCTTTATCGAGGACCTGCT
GTTCAACAAGGTGACCCTGGCCGATGCCGGCTTCATCAAGCAGTATGGCGATTGCCT
GGGCGACATCGCAGCCCGCGACCTGATCTGTGCCCAGAAGTTTAATGGCCTGACCGT
GCTGCCTCCACTGCTGACAGATGAGATGATCGCACAGTACACATCCGCCCTGCTGGC
CGGCACCATCACATCTGGATGGACCTTCGGGGCCGGGGCCGCCCTGCAGATCCCATT
TGCCATGCAGATGGCCTATAGATTCAACGGCATCGGCGTGACCCAGAATGTGCTGTA
CGAGAACCAGAAGCTGATCGCCAATCAGTTTAACTCCGCCATCGGCAAGATCCAGGA
CTCCCTGTCTAGCACAGCCTCTGCCCTGGGCAAGCTGCAGGATGTGGTGAATCAGAA
CGCCCAGGCCCTGAATACCCTGGTGAAGCAGCTCAGCAGCAACTTCGGGGCCATCAG
CAGCGTGCTGAACGACATCCTGAGCCGGCTGGACCCCCCTGAGGCAGAGGTGCAGAT
CGACAGGCTGATCACAGGCCGCCTGCAGAGCCTGCAGACCTACGTGACACAGCAGCT
CATCAGGGCCGCCGAGATCAGAGCCTCCGCCAATCTGGCCGCCACCAAGATGTCTGA
GTGCGTGCTGGGCCAGAGCAAGCGCGTGGACTTTTGTGGCAAGGGCTATCACCTGAT
GTCCTTCCCACAGTCTGCCCCCCACGGAGTGGTGTTTCTGCATGTGACCTACGTGCCT
GCCCAGGAGAAGAACTTCACCACAGCCCCAGCCATCTGCCACGATGGCAAGGCACAC
TTTCCCCGGGAGGGCGTGTTCGTGTCTAACGGCACCCACTGGTTTGTGACACAGAGA
AATTTCTACGAGCCTCAGATCATCACCACAGACAATACATTCGTGAGCGGCAACTGT
GACGTGGTCATCGGCATCGTGAACAATACCGTGTATGATCCCCTGCAGCCTGAGCTG
GACTCCTTTAAGGAGGAGCTGGATAAGTACTTCAAGAATCACACCTCTCCCGACGTG
GATCTGGGCGACATCAGCGGCATCAATGCCTCCGTGGTGAACATCCAGAAGGAGATC
GACAGGCTGAACGAGGTGGCCAAGAATCTGAACGAGTCCCTGATCGATCTGCAGGAG
CTGGGCAAGTATGAGCAGTACATCAAGTGGCCATGGTATATCTGGCTGGGCTTCATC
GCCGGCCTGATCGCCATCGTGATGGTGACCATCATGCTGTGCTGTATGACATCCTGCT
GTTCTTGCCTGAAGGGCTGCTGTAGCTGTGGCTCCTGCTGTAAGTTTGATGAGGACGA
TAGCGAGCCCGTGCTGAAGGGCGTGAAGCTGCACTACACCTGA
SEQ ID NO: 22 is a SARS-CoV-2 “Proline & Furin Cleavage Modified &
VSV-G TMCyt Swap” Spike Glycoprotein, Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPGSASSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDPPEAEVQIDRLITGRLQSLQTYVTQQLIRAAEIRASANLAATKMSECV
LGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQPELDSFKEELD
KYFKNHTSPDVDLGDISGINASVVNIQKEIDRLNEVAKNLNESLIDLQELGKYEQYIKFFFI
IGLIIGLFLVERVGIHECIKLKEITKKRQIYTDIEMNREGK
SEQ ID NO: 23 is a SARS CoV-2 “Proline & Furin Cleavage Modified &
VSV-G TMCyt SWAP” Spike Glycoprotein, Nucleotide Sequence
ATGTTTGTTTTTCTTGTTTTATTGCCACTAGTCTCTAGTCAGTGTGTTAATCTTACAAC
CAGAACTCAATTACCCCCTGCATACACTAATTCTTTCACACGTGGTGTTTATTACCCT
GACAAAGTTTTCAGATCCTCAGTTTTACATTCAACTCAGGACTTGTTCTTACCTTTCTT
TTCCAATGTTACTTGGTTCCATGCTATACATGTCTCTGGGACCAATGGTACTAAGAGG
TTTGATAACCCTGTCCTACCATTTAATGATGGTGTTTATTTTGCTTCCACTGAGAAGTC
TAACATAATAAGAGGCTGGATTTTTGGTACTACTTTAGATTCGAAGACCCAGTCCCTA
CTTATTGTTAATAACGCTACTAATGTTGTTATTAAAGTCTGTGAATTTCAATTTTGTAA
TGATCCATTTTTGGGTGTTTATTACCACAAAAACAACAAAAGTTGGATGGAAAGTGA
GTTCAGAGTTTATTCTAGTGCGAATAATTGCACTTTTGAATATGTCTCTCAGCCTTTTC
TTATGGACCTTGAAGGAAAACAGGGTAATTTCAAAAATCTTAGGGAATTTGTGTTTA
AGAATATTGATGGTTATTTTAAAATATATTCTAAGCACACGCCTATTAATTTAGTGCG
TGATCTCCCTCAGGGTTTTTCGGCTTTAGAACCATTGGTAGATTTGCCAATAGGTATT
AACATCACTAGGTTTCAAACTTTACTTGCTTTACATAGAAGTTATTTGACTCCTGGTG
ATTCTTCTTCAGGTTGGACAGCTGGTGCTGCAGCTTATTATGTGGGTTATCTTCAACCT
AGGACTTTTCTATTAAAATATAATGAAAATGGAACCATTACAGATGCTGTAGACTGT
GCACTTGACCCTCTCTCAGAAACAAAGTGTACGTTGAAATCCTTCACTGTAGAAAAA
GGAATCTATCAAACTTCTAACTTTAGAGTCCAACCAACAGAATCTATTGTTAGATTTC
CTAATATTACAAACTTGTGCCCTTTTGGTGAAGTTTTTAACGCCACCAGATTTGCATCT
GTTTATGCTTGGAACAGGAAGAGAATCAGCAACTGTGTTGCTGATTATTCTGTCCTAT
ATAATTCCGCATCATTTTCCACTTTTAAGTGTTATGGAGTGTCTCCTACTAAATTAAAT
GATCTCTGCTTTACTAATGTCTATGCAGATTCATTTGTAATTAGAGGTGATGAAGTCA
GACAAATCGCTCCAGGGCAAACTGGAAAGATTGCTGATTATAATTATAAATTACCAG
ATGATTTTACAGGCTGCGTTATAGCTTGGAATTCTAACAATCTTGATTCTAAGGTTGG
TGGTAATTATAATTACCTGTATAGATTGTTTAGGAAGTCTAATCTCAAACCTTTTGAG
AGAGATATTTCAACTGAAATCTATCAGGCCGGTAGCACACCTTGTAATGGTGTTGAA
GGTTTTAATTGTTACTTTCCTTTACAATCATATGGTTTCCAACCCACTAATGGTGTTGG
TTACCAACCATACAGAGTAGTAGTACTTTCTTTTGAACTTCTACATGCACCAGCAACT
GTTTGTGGACCTAAAAAGTCTACTAATTTGGTTAAAAACAAATGTGTCAATTTCAACT
TCAATGGTTTAACAGGCACAGGTGTTCTTACTGAGTCTAACAAAAAGTTTCTGCCTTT
CCAACAATTTGGCAGAGACATTGCTGACACTACTGATGCTGTCCGTGATCCACAGAC
ACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAG
TCCCTGTTGCTATTCATGCAGATCAACTTACTCCTACTTGGCGTGTTTATTCTACAGGT
TCTAATGTTTTTCAAACACGTGCAGGCTGTTTAATAGGGGCTGAACATGTCAACAACT
CATATGAGTGTGACATACCCATTGGTGCAGGTATATGCGCTAGTTATCAGACTCAGAC
TAATTCTCCTGGTAGTGCAAGTAGTGTAGCTAGTCAATCCATCATTGCCTACACTATG
TCACTTGGTGCAGAAAATTCAGTTGCTTACTCTAATAACTCTATTGCCATACCCACAA
ATTTTACTATTAGTGTTACCACAGAAATTCTACCAGTGTCTATGACCAAGACATCAGT
AGATTGTACAATGTACATTTGTGGTGATTCAACTGAATGCAGCAATCTTTTGTTGCAA
TATGGCAGTTTTTGTACACAATTAAACCGTGCTTTAACTGGAATAGCTGTTGAACAAG
ACAAAAACACCCAAGAAGTTTTTGCACAAGTCAAACAAATTTACAAAACACCACCAA
TTAAAGATTTTGGTGGTTTTAATTTTTCACAAATATTACCAGATCCATCAAAACCAAG
CAAGAGGTCATTTATTGAAGATCTACTTTTCAACAAAGTGACACTTGCAGATGCTGGC
TTCATCAAACAATATGGTGATTGCCTTGGTGATATTGCTGCTAGAGACCTCATTTGTG
CACAAAAGTTTAACGGCCTTACTGTTTTGCCACCTTTGCTCACAGATGAAATGATTGC
TCAATACACTTCTGCACTGTTAGCGGGTACAATCACTTCTGGTTGGACCTTTGGTGCA
GGTGCTGCATTACAAATACCATTTGCTATGCAAATGGCTTATAGGTTTAATGGTATTG
GAGTTACACAGAATGTTCTCTATGAGAACCAAAAATTGATTGCCAACCAATTTAATA
GTGCTATTGGCAAAATTCAAGACTCACTTTCTTCCACAGCAAGTGCACTTGGAAAACT
TCAAGATGTGGTCAACCAAAATGCACAAGCTTTAAACACGCTTGTTAAACAACTTAG
CTCCAATTTTGGTGCAATTTCAAGTGTTTTAAATGATATCCTTTCACGTCTTGACCCTC
CTGAGGCTGAAGTGCAAATTGATAGGTTGATCACAGGCAGACTTCAAAGTTTGCAGA
CATATGTGACTCAACAATTAATTAGAGCTGCAGAAATCAGAGCTTCTGCTAATCTTGC
TGCTACTAAAATGTCAGAGTGTGTACTTGGACAATCAAAAAGAGTTGATTTTTGTGGA
AAGGGCTATCATCTTATGTCCTTCCCTCAGTCAGCACCTCATGGTGTAGTCTTCTTGCA
TGTGACTTATGTCCCTGCACAAGAAAAGAACTTCACAACTGCTCCTGCCATTTGTCAT
GATGGAAAAGCACACTTTCCTCGTGAAGGTGTCTTTGTTTCAAATGGCACACACTGGT
TTGTAACACAAAGGAATTTTTATGAACCACAAATCATTACTACAGACAACACATTTGT
GTCTGGTAACTGTGATGTTGTAATAGGAATTGTCAACAACACAGTTTATGATCCTTTG
CAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTC
AAAAAGAAATTGACCGCCTCAATGAGGTTGCCAAGAATTTAAATGAATCTCTCATCG
ATCTCCAAGAACTTGGAAAGTATGAGCAGTATATAAAATTTTTCTTTATCATAGGGTT
AATCATTGGACTATTCTTGGTTCTCCGAGTTGGTATCCATCTTTGCATTAAATTAAAGC
ACACCAAGAAAAGACAGATTTATACAGACATAGAGATGAACCGACTTGGAAAGTAA
SEQ ID NO: 24 is a SARS CoV-2 “Proline & Furin Cleavage Modified &
VSV-G TMCyt SWAP” Spike Glycoprotein, Nucleotide Sequence, Codon
Optimized For Expression In Human Cells
ATGTTCGTGTTCCTGGTGCTGCTGCCTCTGGTGAGCTCCCAGTGCGTGAACCTGACCA
CAAGGACCCAGCTCCCCCCTGCCTATACCAATTCCTTTACAAGGGGCGTGTACTATCC
AGACAAGGTGTTCCGCTCTAGCGTGCTGCACTCTACACAGGATCTGTTCCTGCCCTTC
TTTAGCAACGTGACCTGGTTTCACGCCATCCATGTGAGCGGCACCAATGGCACAAAG
CGGTTTGACAATCCTGTGCTGCCATTCAACGATGGCGTGTACTTTGCCTCCACCGAGA
AGTCTAACATCATCAGAGGCTGGATCTTCGGCACCACACTGGACAGCAAGACACAGT
CCCTGCTGATCGTGAACAATGCCACCAACGTGGTCATCAAGGTGTGCGAGTTTCAGTT
CTGTAATGATCCTTTTCTGGGCGTGTACTATCACAAGAACAATAAGTCTTGGATGGAG
AGCGAGTTCCGCGTGTATTCCTCTGCCAACAATTGTACATTCGAGTACGTGTCCCAGC
CATTTCTGATGGACCTGGAGGGCAAGCAGGGCAACTTCAAGAACCTGCGGGAGTTCG
TGTTCAAGAATATCGATGGCTATTTCAAGATCTACTCTAAGCACACCCCTATCAACCT
GGTGCGCGACCTGCCACAGGGCTTTAGCGCCCTGGAGCCTCTGGTGGATCTGCCAAT
CGGCATCAACATCACCAGGTTCCAGACACTGCTGGCCCTGCACCGCAGCTACCTGAC
ACCTGGCGACAGCTCCTCTGGATGGACCGCCGGGGCCGCCGCCTACTATGTGGGCTA
TCTGCAGCCACGGACCTTTCTGCTGAAGTACAACGAGAATGGCACCATCACAGACGC
AGTGGATTGCGCCCTGGACCCCCTGAGCGAGACCAAGTGTACACTGAAGTCCTTCAC
CGTGGAGAAGGGCATCTATCAGACATCCAATTTTCGGGTGCAGCCCACCGAGTCTAT
CGTGAGATTCCCAAATATCACAAACCTGTGCCCCTTCGGCGAGGTGTTTAACGCCACC
AGATTCGCCAGCGTGTACGCCTGGAATCGGAAGAGAATCTCTAACTGCGTGGCCGAC
TATAGCGTGCTGTACAACTCTGCCAGCTTTTCCACCTTCAAGTGCTATGGCGTGTCCC
CCACAAAGCTGAATGACCTGTGCTTCACCAACGTGTACGCCGATTCTTTTGTGATCAG
GGGCGACGAGGTGAGACAGATCGCACCAGGCCAGACAGGCAAGATCGCCGACTACA
ATTATAAGCTGCCCGACGATTTCACCGGCTGCGTGATCGCCTGGAACTCTAACAATCT
GGATAGCAAAGTGGGCGGCAACTACAATTATCTGTACAGGCTGTTCCGCAAGAGCAA
TCTGAAGCCTTTTGAGCGGGACATCTCTACAGAGATCTACCAGGCCGGCAGCACCCC
ATGCAATGGCGTGGAGGGCTTCAACTGTTATTTTCCCCTGCAGTCCTACGGCTTTCAG
CCTACCAACGGCGTGGGCTATCAGCCATACAGAGTGGTGGTGCTGAGCTTCGAGCTG
CTGCACGCACCAGCAACAGTGTGCGGACCTAAGAAGTCCACCAATCTGGTGAAGAAC
AAGTGCGTGAACTTCAACTTCAACGGCCTGACCGGCACAGGCGTGCTGACCGAGTCC
AATAAGAAGTTTCTGCCCTTCCAGCAGTTTGGCCGGGACATCGCCGATACCACAGAC
GCCGTGAGAGACCCCCAGACCCTGGAGATCCTGGACATCACACCTTGCTCCTTCGGC
GGCGTGTCTGTGATCACACCTGGCACCAATACAAGCAACCAGGTGGCCGTGCTGTAT
CAGGACGTGAATTGTACCGAGGTGCCAGTGGCCATCCACGCCGATCAGCTCACCCCC
ACATGGCGGGTGTACTCCACCGGCTCTAACGTGTTCCAGACAAGAGCCGGCTGCCTG
ATCGGAGCCGAGCATGTGAACAATTCCTATGAGTGCGACATCCCCATCGGAGCCGGC
ATCTGTGCCTCTTACCAGACCCAGACAAACAGCCCTGGCTCCGCCAGCTCCGTGGCCT
CTCAGAGCATCATCGCCTATACCATGAGCCTGGGGGCCGAGAACAGCGTGGCCTACT
CTAACAATAGCATCGCCATCCCCACCAACTTTACAATCTCTGTGACCACAGAGATCCT
GCCTGTGAGCATGACCAAGACATCCGTGGACTGCACAATGTATATCTGTGGCGATTC
CACCGAGTGCTCTAACCTGCTGCTGCAGTACGGCAGCTTCTGTACCCAGCTCAACCGG
GCCCTGACAGGAATCGCAGTGGAGCAGGACAAGAACACACAGGAGGTGTTTGCCCA
GGTGAAGCAGATCTACAAGACCCCACCCATCAAGGACTTCGGCGGCTTTAATTTCTCC
CAGATCCTGCCAGATCCCTCCAAGCCATCTAAGCGGAGCTTCATCGAGGACCTGCTGT
TTAACAAGGTGACCCTGGCCGATGCCGGCTTTATCAAGCAGTATGGCGATTGCCTGG
GCGACATCGCCGCCAGAGACCTGATCTGTGCCCAGAAGTTCAATGGCCTGACCGTGC
TGCCTCCACTGCTGACAGATGAGATGATCGCACAGTACACAAGCGCCCTGCTGGCCG
GCACCATCACATCCGGATGGACCTTCGGGGCCGGGGCCGCCCTGCAGATCCCCTTCG
CCATGCAGATGGCCTATAGGTTTAACGGCATCGGCGTGACCCAGAATGTGCTGTACG
AGAACCAGAAGCTGATCGCCAATCAGTTCAACTCCGCCATCGGCAAGATCCAGGACA
GCCTGTCTAGCACAGCCTCCGCCCTGGGCAAGCTGCAGGATGTGGTGAATCAGAACG
CCCAGGCCCTGAATACCCTGGTGAAGCAGCTCAGCAGCAACTTCGGGGCCATCAGCA
GCGTGCTGAACGACATCCTGAGCCGGCTGGACCCCCCTGAGGCAGAGGTGCAGATCG
ACAGGCTGATCACAGGCCGCCTGCAGTCTCTGCAGACCTATGTGACACAGCAGCTCA
TCAGGGCCGCCGAGATCAGAGCCAGCGCCAATCTGGCCGCCACCAAGATGTCCGAGT
GCGTGCTGGGCCAGTCTAAGCGCGTGGACTTCTGTGGCAAGGGCTATCACCTGATGA
GCTTTCCACAGTCCGCCCCCCACGGAGTGGTGTTCCTGCATGTGACCTACGTGCCTGC
CCAGGAGAAGAACTTTACCACAGCCCCAGCCATCTGCCACGATGGCAAGGCACACTT
CCCCAGGGAGGGCGTGTTCGTGAGCAACGGCACCCACTGGTTCGTGACACAGCGCAA
CTTCTACGAGCCTCAGATCATCACCACAGACAATACATTCGTGTCTGGCAACTGTGAC
GTGGTCATCGGCATCGTGAACAATACCGTGTATGATCCCCTGCAGCCTGAGCTGGAC
AGCTTCAAGGAGGAGCTGGATAAGTACTTTAAGAATCACACCTCCCCCGACGTGGAT
CTGGGCGACATCTCTGGCATCAATGCCAGCGTGGTGAACATCCAGAAGGAGATCGAC
AGGCTGAACGAGGTGGCCAAGAATCTGAACGAGAGCCTGATCGATCTGCAGGAGCT
GGGCAAGTATGAGCAGTACATCAAGTTCTTTTTCATCATCGGCCTGATCATCGGCCTG
TTCCTGGTGCTGCGCGTGGGCATCCACCTGTGCATCAAGCTGAAGCACACCAAGAAG
AGGCAGATCTACACAGACATCGAGATGAACCGCCTGGGCAAGTGA
SEQ ID NO: 25 is a SARS-CoV-2 Spike Glycoprotein, with VSV-G TMCyt
SWAP Amino Acid Sequence
MFVFLVLLPLVSSQCVNLTTRTQLPPAYTNSFTRGVYYPDKVFRSSVLHSTQDLFLPFFSN
VTWFHAIHVSGTNGTKRFDNPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVAD
YSVLYNSASFSTFKCYGVSPTKLNDLCFTNVYADSFVIRGDEVRQIAPGQTGKIADYNYK
LPDDFTGCVIAWNSNNLDSKVGGNYNYLYRLFRKSNLKPFERDISTEIYQAGSTPCNGVE
GFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTS
NQVAVLYQDVNCTEVPVAIHADQLTPTWRVYSTGSNVFQTRAGCLIGAEHVNNSYECDI
PIGAGICASYQTQTNSPRRARSVASQSIIAYTMSLGAENSVAYSNNSIAIPTNFTISVTTEILP
VSMTKTSVDCTMYICGDSTECSNLLLQYGSFCTQLNRALTGIAVEQDKNTQEVFAQVKQI
YKTPPIKDFGGFNFSQILPDPSKPSKRSFIEDLLFNKVTLADAGFIKQYGDCLGDIAARDLIC
AQKFNGLTVLPPLLTDEMIAQYTSALLAGTITSGWTFGAGAALQIPFAMQMAYRFNGIGV
TQNVLYENQKLIANQFNSAIGKIQDSLSSTASALGKLQDVVNQNAQALNTLVKQLSSNFG
AISSVLNDILSRLDKVEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIKFF
FIIGLIIGLFLVLRVGIHLCIKLKHTKKRQIYTDIEMNRLGK
SEQ ID NO: 26 is a SARS-CoV-2 Spike Glycoprotein with VSV-G TMCyt
SWAP, Nucleotide Sequence
CTCGAGGTTTAAACGAATTCCGCCACCATGTTTGTTTTTCTTGTTTTATTGCCACTAGT
CTCTAGTCAGTGTGTTAATCTTACAACCAGAACTCAATTACCCCCTGCATACACTAAT
TCTTTCACACGTGGTGTTTATTACCCTGACAAAGTTTTCAGATCCTCAGTTTTACATTC
AACTCAGGACTTGTTCTTACCTTTCTTTTCCAATGTTACTTGGTTCCATGCTATACATG
TCTCTGGGACCAATGGTACTAAGAGGTTTGATAACCCTGTCCTACCATTTAATGATGG
TGTTTATTTTGCTTCCACTGAGAAGTCTAACATAATAAGAGGCTGGATTTTTGGTACT
ACTTTAGATTCGAAGACCCAGTCCCTACTTATTGTTAATAACGCTACTAATGTTGTTA
TTAAAGTCTGTGAATTTCAATTTTGTAATGATCCATTTTTGGGTGTTTATTACCACAAA
AACAACAAAAGTTGGATGGAAAGTGAGTTCAGAGTTTATTCTAGTGCGAATAATTGC
ACTTTTGAATATGTCTCTCAGCCTTTTCTTATGGACCTTGAAGGAAAACAGGGTAATT
TCAAAAATCTTAGGGAATTTGTGTTTAAGAATATTGATGGTTATTTTAAAATATATTC
TAAGCACACGCCTATTAATTTAGTGCGTGATCTCCCTCAGGGTTTTTCGGCTTTAGAA
CCATTGGTAGATTTGCCAATAGGTATTAACATCACTAGGTTTCAAACTTTACTTGCTTT
ACATAGAAGTTATTTGACTCCTGGTGATTCTTCTTCAGGTTGGACAGCTGGTGCTGCA
GCTTATTATGTGGGTTATCTTCAACCTAGGACTTTTCTATTAAAATATAATGAAAATG
GAACCATTACAGATGCTGTAGACTGTGCACTTGACCCTCTCTCAGAAACAAAGTGTA
CGTTGAAATCCTTCACTGTAGAAAAAGGAATCTATCAAACTTCTAACTTTAGAGTCCA
ACCAACAGAATCTATTGTTAGATTTCCTAATATTACAAACTTGTGCCCTTTTGGTGAA
GTTTTTAACGCCACCAGATTTGCATCTGTTTATGCTTGGAACAGGAAGAGAATCAGCA
ACTGTGTTGCTGATTATTCTGTCCTATATAATTCCGCATCATTTTCCACTTTTAAGTGT
TATGGAGTGTCTCCTACTAAATTAAATGATCTCTGCTTTACTAATGTCTATGCAGATTC
ATTTGTAATTAGAGGTGATGAAGTCAGACAAATCGCTCCAGGGCAAACTGGAAAGAT
TGCTGATTATAATTATAAATTACCAGATGATTTTACAGGCTGCGTTATAGCTTGGAAT
TCTAACAATCTTGATTCTAAGGTTGGTGGTAATTATAATTACCTGTATAGATTGTTTA
GGAAGTCTAATCTCAAACCTTTTGAGAGAGATATTTCAACTGAAATCTATCAGGCCG
GTAGCACACCTTGTAATGGTGTTGAAGGTTTTAATTGTTACTTTCCTTTACAATCATAT
GGTTTCCAACCCACTAATGGTGTTGGTTACCAACCATACAGAGTAGTAGTACTTTCTT
TTGAACTTCTACATGCACCAGCAACTGTTTGTGGACCTAAAAAGTCTACTAATTTGGT
TAAAAACAAATGTGTCAATTTCAACTTCAATGGTTTAACAGGCACAGGTGTTCTTACT
GAGTCTAACAAAAAGTTTCTGCCTTTCCAACAATTTGGCAGAGACATTGCTGACACTA
CTGATGCTGTCCGTGATCCACAGACACTTGAGATTCTTGACATTACACCATGTTCTTTT
GGTGGTGTCAGTGTTATAACACCAGGAACAAATACTTCTAACCAGGTTGCTGTTCTTT
ATCAGGATGTTAACTGCACAGAAGTCCCTGTTGCTATTCATGCAGATCAACTTACTCC
TACTTGGCGTGTTTATTCTACAGGTTCTAATGTTTTTCAAACACGTGCAGGCTGTTTAA
TAGGGGCTGAACATGTCAACAACTCATATGAGTGTGACATACCCATTGGTGCAGGTA
TATGCGCTAGTTATCAGACTCAGACTAATTCTCCTCGGCGGGCACGTAGTGTAGCTAG
TCAATCCATCATTGCCTACACTATGTCACTTGGTGCAGAAAATTCAGTTGCTTACTCT
AATAACTCTATTGCCATACCCACAAATTTTACTATTAGTGTTACCACAGAAATTCTAC
CAGTGTCTATGACCAAGACATCAGTAGATTGTACAATGTACATTTGTGGTGATTCAAC
TGAATGCAGCAATCTTTTGTTGCAATATGGCAGTTTTTGTACACAATTAAACCGTGCT
TTAACTGGAATAGCTGTTGAACAAGACAAAAACACCCAAGAAGTTTTTGCACAAGTC
AAACAAATTTACAAAACACCACCAATTAAAGATTTTGGTGGTTTTAATTTTTCACAAA
TATTACCAGATCCATCAAAACCAAGCAAGAGGTCATTTATTGAAGATCTACTTTTCAA
CAAAGTGACACTTGCAGATGCTGGCTTCATCAAACAATATGGTGATTGCCTTGGTGAT
ATTGCTGCTAGAGACCTCATTTGTGCACAAAAGTTTAACGGCCTTACTGTTTTGCCAC
CTTTGCTCACAGATGAAATGATTGCTCAATACACTTCTGCACTGTTAGCGGGTACAAT
CACTTCTGGTTGGACCTTTGGTGCAGGTGCTGCATTACAAATACCATTTGCTATGCAA
ATGGCTTATAGGTTTAATGGTATTGGAGTTACACAGAATGTTCTCTATGAGAACCAAA
AATTGATTGCCAACCAATTTAATAGTGCTATTGGCAAAATTCAAGACTCACTTTCTTC
CACAGCAAGTGCACTTGGAAAACTTCAAGATGTGGTCAACCAAAATGCACAAGCTTT
AAACACGCTTGTTAAACAACTTAGCTCCAATTTTGGTGCAATTTCAAGTGTTTTAAAT
GATATCCTTTCACGTCTTGACAAAGTTGAGGCTGAAGTGCAAATTGATAGGTTGATCA
CAGGCAGACTTCAAAGTTTGCAGACATATGTGACTCAACAATTAATTAGAGCTGCAG
AAATCAGAGCTTCTGCTAATCTTGCTGCTACTAAAATGTCAGAGTGTGTACTTGGACA
ATCAAAAAGAGTTGATTTTTGTGGAAAGGGCTATCATCTTATGTCCTTCCCTCAGTCA
GCACCTCATGGTGTAGTCTTCTTGCATGTGACTTATGTCCCTGCACAAGAAAAGAACT
TCACAACTGCTCCTGCCATTTGTCATGATGGAAAAGCACACTTTCCTCGTGAAGGTGT
CTTTGTTTCAAATGGCACACACTGGTTTGTAACACAAAGGAATTTTTATGAACCACAA
ATCATTACTACAGACAACACATTTGTGTCTGGTAACTGTGATGTTGTAATAGGAATTG
TCAACAACACAGTTTATGATCCTTTGCAACCTGAATTAGACTCATTCAAGGAGGAGTT
AGATAAATATTTTAAGAATCATACATCACCAGATGTTGATTTAGGTGACATCTCTGGC
ATTAATGCTTCAGTTGTAAACATTCAAAAAGAAATTGACCGCCTCAATGAGGTTGCC
AAGAATTTAAATGAATCTCTCATCGATCTCCAAGAACTTGGAAAGTATGAGCAGTAT
ATAAAATGGCCATTTTTCTTTATCATAGGGTTAATCATTGGACTATTCTTGGTTCTCCG
AGTTGGTATCCATCTTTGCATTAAATTAAAGCACACCAAGAAAAGACAGATTTATAC
AGACATAGAGATGAACCGACTTGGAAAGTAAGAATTCCACGTGGGATCC
SEQ ID NO: 27 is a SARS CoV-2 Spike Glycoprotein with VSV-G TMCyt
SWAP, Nucleotide Sequence, Codon Optimized For Expression In Human Cells
CTCGAGGTTTAAACGAATTCCGCCACCATGTTCGTGTTCCTGGTGCTGCTGCCCCTGG
TGTCCAGCCAGTGCGTGAATCTGACCACCCGGACCCAACTGCCTCCCGCCTACACAA
ACTCTTTCACCAGAGGGGTTTATTACCCCGATAAGGTGTTCAGAAGCTCAGTGCTTCA
TTCTACCCAGGACCTGTTTCTGCCTTTTTTCAGCAACGTCACATGGTTCCACGCCATCC
ACGTCAGCGGAACCAACGGCACGAAGCGGTTCGACAATCCTGTGCTGCCTTTTAACG
ACGGCGTCTACTTTGCCAGCACGGAAAAGAGCAACATTATCCGGGGATGGATCTTCG
GCACCACCCTGGACTCTAAAACCCAGAGCCTGTTGATCGTGAACAACGCAACCAATG
TGGTGATCAAGGTCTGCGAGTTCCAATTTTGCAACGATCCTTTCCTGGGCGTGTACTA
CCACAAGAACAACAAGTCTTGGATGGAATCTGAGTTCCGCGTCTACAGCAGCGCAAA
CAACTGCACATTTGAGTACGTGTCTCAGCCTTTTCTGATGGACCTGGAAGGAAAGCA
GGGAAATTTCAAGAACCTGCGGGAGTTCGTGTTCAAGAACATCGACGGCTACTTCAA
GATCTACAGCAAGCACACCCCCATCAACCTCGTGAGAGACCTGCCCCAGGGCTTCAG
CGCCCTGGAACCCCTGGTGGACCTTCCCATAGGAATCAACATCACACGGTTCCAGAC
ACTGCTGGCCCTGCATAGAAGCTACCTGACCCCTGGAGATTCTAGCAGCGGCTGGAC
CGCCGGCGCTGCCGCTTACTACGTCGGATACCTGCAGCCTAGAACCTTCCTGTTGAAG
TACAACGAGAACGGCACCATCACAGATGCCGTGGACTGCGCCCTGGACCCCCTGAGC
GAGACAAAGTGCACCCTGAAGAGCTTCACCGTGGAGAAGGGCATCTACCAGACAAG
CAACTTCAGAGTGCAGCCTACCGAGTCAATCGTGAGATTCCCAAACATCACCAACCT
TTGTCCTTTCGGCGAGGTATTTAACGCCACCCGGTTCGCCAGCGTGTACGCCTGGAAT
AGGAAGCGGATCAGCAACTGCGTGGCCGATTACAGCGTGCTCTATAACAGCGCCAGT
TTTAGCACTTTCAAGTGCTACGGAGTCTCTCCTACAAAGCTGAACGACCTGTGCTTCA
CCAACGTGTATGCCGACAGCTTCGTCATCCGGGGCGACGAGGTGCGACAGATCGCTC
CTGGCCAGACCGGCAAGATAGCCGACTACAACTACAAGCTGCCTGACGACTTCACAG
GCTGCGTGATCGCTTGGAACAGCAACAATCTGGATAGCAAAGTGGGCGGCAACTATA
ACTACCTGTACAGACTGTTCCGGAAGTCCAATCTCAAGCCGTTTGAGAGAGACATCA
GCACCGAAATCTACCAGGCTGGATCTACACCCTGCAACGGCGTCGAAGGCTTCAATT
GTTACTTCCCTCTGCAATCTTACGGCTTCCAGCCCACCAACGGCGTGGGCTACCAGCC
CTACAGAGTGGTTGTGCTGAGCTTCGAGCTGCTGCACGCCCCAGCTACAGTGTGCGG
CCCTAAGAAATCTACAAACCTGGTCAAGAACAAGTGTGTGAACTTCAACTTCAATGG
CCTGACGGGCACCGGCGTGCTGACAGAGAGCAACAAGAAGTTCCTGCCTTTCCAGCA
ATTTGGCAGAGACATCGCCGACACCACCGACGCCGTGCGCGACCCTCAGACCCTGGA
AATTCTGGACATCACCCCATGTTCTTTCGGCGGCGTGTCCGTCATTACGCCAGGCACC
AATACCAGCAACCAGGTGGCCGTGCTTTATCAGGATGTGAATTGTACCGAAGTTCCT
GTTGCAATCCACGCCGACCAACTGACCCCCACATGGAGAGTGTACTCTACCGGCAGC
AACGTGTTCCAAACGAGAGCCGGATGCCTGATTGGAGCTGAGCATGTGAACAACAGC
TACGAGTGCGATATTCCAATCGGAGCCGGCATCTGCGCCTCCTACCAAACACAAACC
AACTCCCCTCGTAGAGCGAGAAGCGTGGCCTCTCAGAGCATCATCGCCTACACCATG
AGCCTGGGTGCCGAAAACTCCGTGGCTTACTCCAACAACAGCATCGCCATCCCTACA
AATTTCACCATCAGCGTGACAACCGAGATCCTGCCTGTGTCCATGACCAAGACCAGC
GTGGACTGCACGATGTACATCTGCGGAGATAGCACCGAGTGCAGCAATCTGCTACTG
CAGTATGGCAGCTTCTGCACCCAACTGAACAGAGCACTGACCGGCATTGCTGTGGAA
CAGGACAAGAATACCCAGGAGGTGTTCGCCCAAGTGAAGCAGATTTACAAGACACCC
CCTATCAAGGACTTCGGAGGCTTCAACTTCAGCCAGATCCTGCCTGACCCTAGCAAGC
CAAGCAAAAGATCCTTTATCGAAGATCTGCTGTTTAACAAGGTGACACTGGCCGATG
CCGGCTTTATCAAGCAGTACGGCGACTGCCTGGGAGACATCGCCGCCAGAGACCTGA
TCTGTGCTCAGAAATTTAACGGGCTGACCGTGCTGCCACCTCTGCTGACAGATGAGAT
GATCGCTCAGTACACCAGCGCCCTGCTGGCCGGCACAATTACCTCCGGCTGGACCTTC
GGAGCCGGAGCCGCCCTGCAGATCCCCTTCGCCATGCAGATGGCCTACCGGTTCAAT
GGCATCGGCGTCACCCAAAACGTGCTCTATGAGAACCAGAAGCTGATCGCAAACCAG
TTCAACTCCGCCATCGGTAAGATCCAGGACAGTCTGAGCAGCACGGCGTCTGCCCTG
GGCAAGCTCCAGGACGTGGTGAACCAGAACGCCCAGGCCCTTAACACCCTGGTGAAA
CAACTGAGCAGCAACTTCGGTGCCATTTCCAGCGTTCTCAATGACATCCTGAGCAGAC
TGGATAAGGTGGAAGCCGAGGTGCAGATCGACCGGCTGATCACCGGACGGCTGCAG
AGCCTGCAGACGTACGTGACCCAGCAATTAATCAGAGCTGCCGAGATCAGAGCCAGC
GCCAATCTGGCTGCCACCAAAATGAGCGAATGTGTGCTGGGCCAGTCAAAGAGAGTG
GATTTTTGTGGCAAAGGCTACCACCTGATGTCCTTCCCTCAGTCTGCCCCTCACGGCG
TGGTGTTCCTCCATGTGACCTATGTGCCTGCTCAGGAGAAGAACTTTACCACAGCCCC
TGCTATCTGCCACGACGGAAAGGCCCACTTCCCCAGAGAGGGCGTGTTTGTGTCCAA
TGGCACACACTGGTTCGTGACCCAAAGAAACTTCTACGAGCCCCAGATCATCACCAC
AGACAACACCTTCGTGAGCGGCAACTGCGACGTGGTGATCGGCATCGTGAACAACAC
AGTGTACGACCCCCTGCAACCTGAGCTGGACAGCTTCAAAGAGGAACTGGACAAATA
CTTCAAGAATCACACCAGCCCTGATGTGGATCTGGGCGACATCAGCGGCATCAACGC
CAGCGTCGTGAACATCCAGAAGGAAATCGACAGACTGAACGAAGTGGCCAAGAACC
TGAACGAGAGCCTCATCGATCTGCAGGAGCTGGGCAAGTACGAGCAGTACATCAAAT
GGCCTTTCTTCTTCATCATCGGCCTGATTATCGGCCTGTTCCTCGTGCTGAGAGTGGGC
ATCCACCTGTGCATCAAGCTTAAGCACACAAAAAAGCGGCAGATTTACACCGACATC
GAGATGAACCGGCTGGGCAAATGAGAATTCCACGTGGGATCC
SEQ ID NO: 28 is a SARS-CoV-2 Beta Variant Spike Glycoprotein, with
VSV-G TMCyt SWAP Amino Acid Sequence
MFVFLVLLPLVSSQCVNFTTRTQLPPAYTNSFTRGVYYPDKVFRSSVLHSTQDLFLPFFSN
VTWFHAIHVSGTNGTKRFANPVLPFNDGVYFASTEKSNIIRGWIFGTTLDSKTQSLLIVNN
ATNVVIKVCEFQFCNDPFLGVYYHKNNKSWMESEFRVYSSANNCTFEYVSQPFLMDLEG
KQGNFKNLREFVFKNIDGYFKIYSKHTPINLVRGLPQGFSALEPLVDLPIGINITRFQTLHRS
YLTPGDSSSGWTAGAAAYYVGYLQPRTFLLKYNENGTITDAVDCALDPLSETKCTLKSFT
VEKGIYQTSNFRVQPTESIVRFPNITNLCPFGEVFNATRFASVYAWNRKRISNCVADYSVL
YNSASFSTFKCYGVSPTKLNDLCFTNVYADSFVIRGDEVRQIAPGQTGNIADYNYKLPDD
FTGCVIAWNSNNLDSKVGGNYNYLYRLFRKSNLKPFERDISTEIYQAGSTPCNGVKGFNC
YFPLQSYGFQPTYGVGYQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVA
VLYQGVNCTEVPVAIHADQLTPTWRVYSTGSNVFQTRAGCLIGAEHVNNSYECDIPIGAG
ICASYQTQTNSPGSASSVASQSIIAYTMSLGVENSVAYSNNSIAIPTNFTISVTTEILPVSMT
KTSVDCTMYICGDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCLGDIAARDLICAQK
FNGLTVLPPLLTDEMIAQYTSALLAGTITSGWTFGAGAALQIPFAMQMAYRFNGIGVTQN
VLYENQKLIANQFNSAIGKIQDSLSSTASALGKLQDVVNQNAQALNTLVKQLSSNFGAISS
VLNDILSRLDPPEAEVQIDRLITGRLQSLQTYVTQQLIRAAEIRASANLAATKMSECVLGQ
SKRVDFCGKGYHLMSFPQSAPHGVVFLHVTYVPAQEKNFTTAPAICHDGKAHFPREGVF
VSNGTHWFVTQRNFYEPQIITTDNTFVSGNCDVVIGIVNNTVYDPLQPELDSFKEELDKYF
KNHTSPDVDLGDISGINASVVNIQKEIDRLNEVAKNLNESLIDLQELGKYEQYIKFFFIIGLI
IGLFLVLRVGIHLCIKLKHTKKRQIYTDIEMNRLGK
SEQ ID NO: 29 is a SARS-CoV-2 Beta Variant Spike Glycoprotein with VSV-
G TMCyt SWAP, Nucleotide Sequence
ATGTTTGTTTTTCTTGTTTTATTGCCACTAGTCTCTAGTCAGTGTGTTAATTTTACAACC
AGAACTCAATTACCCCCTGCATACACTAATTCTTTCACACGTGGTGTTTATTACCCTG
ACAAAGTTTTCAGATCCTCAGTTTTACATTCAACTCAGGACTTGTTCTTACCTTTCTTT
TCCAATGTTACTTGGTTCCATGCTATACATGTCTCTGGGACCAATGGTACTAAGAGGT
TTGCTAACCCTGTCCTACCATTTAATGATGGTGTTTATTTTGCTTCCACTGAGAAGTCT
AACATAATAAGAGGCTGGATTTTTGGTACTACTTTAGATTCGAAGACCCAGTCCCTAC
TTATTGTTAATAACGCTACTAATGTTGTTATTAAAGTCTGTGAATTTCAATTTTGTAAT
GATCCATTTTTGGGTGTTTATTACCACAAAAACAACAAAAGTTGGATGGAAAGTGAG
TTCAGAGTTTATTCTAGTGCGAATAATTGCACTTTTGAATATGTCTCTCAGCCTTTTCT
TATGGACCTTGAAGGAAAACAGGGTAATTTCAAAAATCTTAGGGAATTTGTGTTTAA
GAATATTGATGGTTATTTTAAAATATATTCTAAGCACACGCCTATTAATTTAGTGCGT
GGTCTCCCTCAGGGTTTTTCGGCTTTAGAACCATTGGTAGATTTGCCAATAGGTATTA
ACATCACTAGGTTTCAAACTTTACATAGAAGTTATTTGACTCCTGGTGATTCTTCTTCA
GGTTGGACAGCTGGTGCTGCAGCTTATTATGTGGGTTATCTTCAACCTAGGACTTTTC
TATTAAAATATAATGAAAATGGAACCATTACAGATGCTGTAGACTGTGCACTTGACC
CTCTCTCAGAAACAAAGTGTACGTTGAAATCCTTCACTGTAGAAAAAGGAATCTATC
AAACTTCTAACTTTAGAGTCCAACCAACAGAATCTATTGTTAGATTTCCTAATATTAC
AAACTTGTGCCCTTTTGGTGAAGTTTTTAACGCCACCAGATTTGCATCTGTTTATGCTT
GGAACAGGAAGAGAATCAGCAACTGTGTTGCTGATTATTCTGTCCTATATAATTCCGC
ATCATTTTCCACTTTTAAGTGTTATGGAGTGTCTCCTACTAAATTAAATGATCTCTGCT
TTACTAATGTCTATGCAGATTCATTTGTAATTAGAGGTGATGAAGTCAGACAAATCGC
TCCAGGGCAAACTGGAAATATTGCTGATTATAATTATAAATTACCAGATGATTTTACA
GGCTGCGTTATAGCTTGGAATTCTAACAATCTTGATTCTAAGGTTGGTGGTAATTATA
ATTACCTGTATAGATTGTTTAGGAAGTCTAATCTCAAACCTTTTGAGAGAGATATTTC
AACTGAAATCTATCAGGCCGGTAGCACACCTTGTAATGGTGTTAAAGGTTTTAATTGT
TACTTTCCTTTACAATCATATGGTTTCCAACCCACTTATGGTGTTGGTTACCAACCATA
CAGAGTAGTAGTACTTTCTTTTGAACTTCTACATGCACCAGCAACTGTTTGTGGACCT
AAAAAGTCTACTAATTTGGTTAAAAACAAATGTGTCAATTTCAACTTCAATGGTTTAA
CAGGCACAGGTGTTCTTACTGAGTCTAACAAAAAGTTTCTGCCTTTCCAACAATTTGG
CAGAGACATTGCTGACACTACTGATGCTGTCCGTGATCCACAGACACTTGAGATTCTT
GACATTACACCATGTTCTTTTGGTGGTGTCAGTGTTATAACACCAGGAACAAATACTT
CTAACCAGGTTGCTGTTCTTTATCAGGGTGTTAACTGCACAGAAGTCCCTGTTGCTAT
TCATGCAGATCAACTTACTCCTACTTGGCGTGTTTATTCTACAGGTTCTAATGTTTTTC
AAACACGTGCAGGCTGTTTAATAGGGGCTGAACATGTCAACAACTCATATGAGTGTG
ACATACCCATTGGTGCAGGTATATGCGCTAGTTATCAGACTCAGACTAATTCTCCTGG
TAGTGCAAGTAGTGTAGCTAGTCAATCCATCATTGCCTACACTATGTCACTTGGTGTA
GAAAATTCAGTTGCTTACTCTAATAACTCTATTGCCATACCCACAAATTTTACTATTA
GTGTTACCACAGAAATTCTACCAGTGTCTATGACCAAGACATCAGTAGATTGTACAAT
GTACATTTGTGGTGATTCAACTGAATGCAGCAATCTTTTGTTGCAATATGGCAGTTTT
TGTACACAATTAAACCGTGCTTTAACTGGAATAGCTGTTGAACAAGACAAAAACACC
CAAGAAGTTTTTGCACAAGTCAAACAAATTTACAAAACACCACCAATTAAAGATTTT
GGTGGTTTTAATTTTTCACAAATATTACCAGATCCATCAAAACCAAGCAAGAGGTCAT
TTATTGAAGATCTACTTTTCAACAAAGTGACACTTGCAGATGCTGGCTTCATCAAACA
ATATGGTGATTGCCTTGGTGATATTGCTGCTAGAGACCTCATTTGTGCACAAAAGTTT
AACGGCCTTACTGTTTTGCCACCTTTGCTCACAGATGAAATGATTGCTCAATACACTT
CTGCACTGTTAGCGGGTACAATCACTTCTGGTTGGACCTTTGGTGCAGGTGCTGCATT
ACAAATACCATTTGCTATGCAAATGGCTTATAGGTTTAATGGTATTGGAGTTACACAG
AATGTTCTCTATGAGAACCAAAAATTGATTGCCAACCAATTTAATAGTGCTATTGGCA
AAATTCAAGACTCACTTTCTTCCACAGCAAGTGCACTTGGAAAACTTCAAGATGTGGT
CAACCAAAATGCACAAGCTTTAAACACGCTTGTTAAACAACTTAGCTCCAATTTTGGT
GCAATTTCAAGTGTTTTAAATGATATCCTTTCACGTCTTGACCCTCCTGAGGCTGAAG
TGCAAATTGATAGGTTGATCACAGGCAGACTTCAAAGTTTGCAGACATATGTGACTC
AACAATTAATTAGAGCTGCAGAAATCAGAGCTTCTGCTAATCTTGCTGCTACTAAAAT
GTCAGAGTGTGTACTTGGACAATCAAAAAGAGTTGATTTTTGTGGAAAGGGCTATCA
TCTTATGTCCTTCCCTCAGTCAGCACCTCATGGTGTAGTCTTCTTGCATGTGACTTATG
TCCCTGCACAAGAAAAGAACTTCACAACTGCTCCTGCCATTTGTCATGATGGAAAAG
CACACTTTCCTCGTGAAGGTGTCTTTGTTTCAAATGGCACACACTGGTTTGTAACACA
AAGGAATTTTTATGAACCACAAATCATTACTACAGACAACACATTTGTGTCTGGTAAC
TGTGATGTTGTAATAGGAATTGTCAACAACACAGTTTATGATCCTTTGCAACCTGAAT
TAGACTCATTCAAGGAGGAGTTAGATAAATATTTTAAGAATCATACATCACCAGATG
TTGATTTAGGTGACATCTCTGGCATTAATGCTTCAGTTGTAAACATTCAAAAAGAAAT
TGACCGCCTCAATGAGGTTGCCAAGAATTTAAATGAATCTCTCATCGATCTCCAAGAA
CTTGGAAAGTATGAGCAGTATATAAAATTTTTCTTTATCATAGGGTTAATCATTGGAC
TATTCTTGGTTCTCCGAGTTGGTATCCATCTTTGCATTAAATTAAAGCACACCAAGAA
AAGACAGATTTATACAGACATAGAGATGAACCGACTTGGAAAGTAA
SEQ ID NO: 30 is a SARS CoV-2 Beta Variant Spike Glycoprotein with VSV-G
TMCyt SWAP, Nucleotide Sequence, Codon Optimized For Expression In Human
Cells
ATGTTCGTGTTCCTGGTGCTGCTGCCTCTGGTGAGCTCCCAGTGCGTGAACTTCACCA
CAAGGACCCAGCTCCCCCCTGCCTACACCAATTCCTTTACAAGGGGCGTGTACTATCC
AGACAAGGTGTTCCGCTCTAGCGTGCTGCACTCTACACAGGATCTGTTCCTGCCCTTC
TTTAGCAACGTGACCTGGTTTCACGCCATCCATGTGAGCGGCACCAATGGCACAAAG
CGGTTTGCCAATCCTGTGCTGCCATTCAACGACGGCGTGTATTTTGCCTCCACCGAGA
AGTCTAACATCATCAGAGGCTGGATCTTCGGCACCACACTGGATAGCAAGACACAGT
CCCTGCTGATCGTGAACAATGCCACCAACGTGGTCATCAAGGTGTGCGAGTTTCAGTT
CTGTAATGACCCTTTTCTGGGCGTGTACTATCACAAGAACAATAAGTCTTGGATGGAG
AGCGAGTTCCGCGTGTACTCCTCTGCCAACAATTGTACATTCGAGTACGTGAGCCAGC
CATTTCTGATGGACCTGGAGGGCAAGCAGGGCAACTTCAAGAACCTGCGGGAGTTCG
TGTTCAAGAATATCGATGGCTATTTCAAGATCTACTCTAAGCACACCCCTATCAACCT
GGTGCGCGGACTGCCACAGGGCTTTAGCGCCCTGGAGCCTCTGGTGGACCTGCCAAT
CGGCATCAACATCACCAGGTTCCAGACACTGCACCGCAGCTACCTGACACCTGGCGA
TAGCTCCTCTGGATGGACCGCCGGGGCCGCCGCCTACTATGTGGGCTACCTGCAGCC
ACGGACCTTTCTGCTGAAGTATAACGAGAATGGCACCATCACAGACGCAGTGGATTG
CGCCCTGGACCCCCTGAGCGAGACCAAGTGTACACTGAAGTCCTTCACCGTGGAGAA
GGGCATCTACCAGACATCCAATTTTCGGGTGCAGCCCACCGAGTCTATCGTGAGATTC
CCAAATATCACAAACCTGTGCCCCTTCGGCGAGGTGTTTAACGCCACCAGATTCGCCA
GCGTGTATGCCTGGAATCGGAAGAGAATCTCTAACTGCGTGGCCGACTATAGCGTGC
TGTACAACTCTGCCAGCTTTTCCACCTTCAAGTGCTATGGCGTGTCCCCCACAAAGCT
GAATGACCTGTGCTTCACCAACGTGTATGCCGATTCTTTTGTGATCAGGGGCGACGAG
GTGAGACAGATCGCACCAGGCCAGACAGGCAATATCGCCGACTACAACTATAAGCTG
CCCGACGATTTCACCGGCTGCGTGATCGCCTGGAACAGCAACAATCTGGATAGCAAA
GTGGGCGGCAACTACAATTATCTGTACAGGCTGTTCCGCAAGAGCAACCTGAAGCCT
TTTGAGCGGGACATCTCTACAGAGATCTACCAGGCCGGCAGCACCCCATGCAATGGC
GTGAAGGGCTTCAACTGTTATTTTCCCCTGCAGTCCTACGGCTTTCAGCCTACCTATG
GCGTGGGCTATCAGCCATACAGAGTGGTGGTGCTGAGCTTCGAGCTGCTGCACGCAC
CAGCAACAGTGTGCGGACCTAAGAAGTCCACCAATCTGGTGAAGAACAAGTGCGTGA
ACTTCAACTTCAACGGCCTGACCGGCACAGGCGTGCTGACCGAGTCCAATAAGAAGT
TTCTGCCCTTCCAGCAGTTTGGCCGGGACATCGCCGATACCACAGACGCCGTGAGAG
ACCCCCAGACCCTGGAGATCCTGGACATCACACCTTGCTCCTTTGGCGGCGTGTCTGT
GATCACACCTGGCACCAATACAAGCAACCAGGTGGCCGTGCTGTACCAGGGCGTGAA
TTGTACCGAGGTGCCAGTGGCCATCCACGCAGACCAGCTCACCCCCACATGGCGGGT
GTATTCCACCGGCTCTAACGTGTTCCAGACAAGAGCCGGCTGCCTGATCGGAGCCGA
GCATGTGAACAATTCCTACGAGTGCGACATCCCCATCGGAGCCGGCATCTGTGCCTCT
TATCAGACCCAGACAAACAGCCCTGGCTCCGCCAGCTCCGTGGCCTCTCAGAGCATC
ATCGCCTACACCATGTCCCTGGGCGTGGAGAACAGCGTGGCCTATTCTAACAATAGC
ATCGCCATCCCCACCAACTTTACAATCTCTGTGACCACAGAGATCCTGCCTGTGAGCA
TGACCAAGACATCCGTGGACTGCACAATGTACATCTGTGGCGATTCCACCGAGTGCT
CTAACCTGCTGCTGCAGTATGGCAGCTTCTGTACCCAGCTCAACCGGGCCCTGACAGG
AATCGCAGTGGAGCAGGACAAGAACACACAGGAGGTGTTTGCCCAGGTGAAGCAGA
TCTACAAGACCCCACCCATCAAGGACTTCGGCGGCTTTAATTTCTCCCAGATCCTGCC
AGATCCCTCCAAGCCATCTAAGCGGAGCTTCATCGAGGACCTGCTGTTTAACAAGGT
GACCCTGGCCGATGCCGGCTTTATCAAGCAGTACGGCGATTGCCTGGGCGACATCGC
AGCCAGAGACCTGATCTGTGCCCAGAAGTTCAATGGCCTGACCGTGCTGCCTCCACT
GCTGACAGATGAGATGATCGCCCAGTATACAAGCGCCCTGCTGGCCGGCACCATCAC
ATCCGGATGGACCTTCGGGGCCGGGGCCGCCCTGCAGATCCCCTTCGCCATGCAGAT
GGCCTACAGGTTTAACGGCATCGGCGTGACCCAGAATGTGCTGTATGAGAACCAGAA
GCTGATCGCCAATCAGTTCAACTCCGCCATCGGCAAGATCCAGGACAGCCTGTCTAG
CACAGCCTCCGCCCTGGGCAAGCTGCAGGATGTGGTGAATCAGAACGCCCAGGCCCT
GAATACCCTGGTGAAGCAGCTCAGCAGCAACTTCGGGGCCATCAGCAGCGTGCTGAA
CGACATCCTGAGCCGGCTGGACCCCCCTGAGGCAGAGGTGCAGATCGACAGGCTGAT
CACAGGCCGCCTGCAGTCTCTGCAGACCTACGTGACACAGCAGCTCATCAGGGCCGC
CGAGATCAGAGCCAGCGCCAATCTGGCCGCCACCAAGATGTCCGAGTGCGTGCTGGG
CCAGTCTAAGCGCGTGGACTTCTGTGGCAAGGGCTACCACCTGATGAGCTTTCCACA
GTCCGCCCCCCACGGAGTGGTGTTCCTGCATGTGACCTATGTGCCTGCCCAGGAGAA
GAACTTTACCACAGCCCCAGCCATCTGCCACGATGGCAAGGCACACTTCCCCAGGGA
GGGCGTGTTCGTGAGCAACGGCACCCACTGGTTCGTGACACAGCGCAACTTCTACGA
GCCTCAGATCATCACCACAGACAATACATTCGTGTCTGGCAACTGTGACGTGGTCATC
GGCATCGTGAACAATACCGTGTACGATCCCCTGCAGCCTGAGCTGGACAGCTTCAAG
GAGGAGCTGGATAAGTACTTCAAGAATCACACCTCCCCCGACGTGGATCTGGGCGAC
ATCTCTGGCATCAATGCCAGCGTGGTGAACATCCAGAAGGAGATCGACAGGCTGAAC
GAGGTGGCCAAGAATCTGAACGAGAGCCTGATCGATCTGCAGGAGCTGGGCAAGTAT
GAGCAGTACATCAAGTTCTTTTTCATCATCGGCCTGATCATCGGCCTGTTCCTGGTGC
TGCGCGTGGGCATCCACCTGTGCATCAAGCTGAAGCACACCAAGAAGAGGCAGATCT
ACACAGACATCGAGATGAACCGCCTGGGCAAGTGA

### DETAILED DESCRIPTION OF THE EMBODIMENTS

Coronaviruses, such as SARS-CoV, MERS-CoV and SARS-Cov-2, are enveloped viruses having an RNA genome of about 30,000 bases. They fall within the beta genus of coronaviruses. They contain a nucleocapsid surrounded by a lipid bilayer derived from the host cell. An envelope-anchored spike protein (called “S”) mediates the entry of the coronavirus into host cells by binding a host receptor and then fusing viral and host membranes. A defined RBD is the receptor for angiotensin converting enzyme 2 (ACE2). (Wan et al., J. Vir. (2020) 94: 1). Coronavirus S proteins contain three copies of an 51 subunit and three copies of an S2 subunit. Coronavirus S proteins are cleaved into 51 and S2 subunits by furin during protein biosynthesis. The two subunits trimerize and fold into a metastable prefusion conformation. The 51 subunit is responsible for receptor binding while the S2 subunit mediates membrane fusion.

SARS-CoV and SARS-CoV-2 spike protein share about 76% sequence homology, suggesting that these two viruses share the same receptor, ACE2. There is lower sequence similarity between SARS-CoV-2 and MERS-CoV.

Studies on the genomes of SARS-CoV-2 isolated from patients over the span of four months from December 2019 to March 2020 showed that the overall similarity of the human strains declined over the four month period indicating mutation of the virus had occurred within the human population to 0.988468, corresponding to an average of 348.33 nucleotide differences. Such changes imply evolutional changes of this virus, which might result in attenuation or more virulent strains (Li et al 2020. Xidan University). Subsequently, the viral variant which was predominant prior to March 2020, D614, was overtaken by another variant which has a single amino acid change to the spike protein, G614, even in areas where D614 was well established (Korber et al, (2020) Cell, 4:812-827). Subsequently, in late 2020, an unexpected rise in reported COVID-19 cases was attributed to the emergence of the new variants, Alpha (B.1.1.7) in the UK and Beta (501Y.V2) in South Africa (Fontanet et al, (2021) the Lancet, 397: 952-954). Both variants have a mutation (N501Y) in the RBD of the spike protein that is reported to contribute to increased transmission, with estimates ranging between 40% and 70% for increased transmission. The Beta variant has two additional mutations (E484K and K417N) in the spike protein that confer a potential immune escape to antibodies. A further variant of concern, Gamma, emerged in Brazil with another set of mutations (N501Y, E484K, and K417T). The Delta variant includes RBD mutation L452R which contributes to escape from neutralization by antibodies induced by previously acquired immunity and mutation P681R in the furin cleavage site which is related to an increase the rate of S1-S2 cleavage, resulting in better transmissibility. Emergence of escape mutants is a major concern because most of the licensed vaccines against COVID-19 are based on the sequence of spike protein from the ancestral Wuhan strain of SARS-CoV-2.

An important concern is whether the currently available COVID-19 vaccines will be able to protect against infection or disease from the SARS-CoV-2 variants. Preliminary research suggests sera from individuals immunized with the mRNA COVID-19 vaccines neutralized a pseudovirus analogous to the U.K. variant but were less effective against a pseudovirus analogous to the South Africa variant (Yang et al (2021) Nature, doi.org/10.1038/s41586-021-03324-6). Moreover, preliminary results of studies using viral vector vaccines demonstrated good efficacy against the UK variant but poor efficacy against the South Africa variant (Madhi et al (2021) N.E.J.M. DOI: 10.1056/NEJMoa2102214). Therefore, it appears that a vaccine which is capable of inducing production of broadly reactive antibodies would be required to provide protection from infection by variant strains of coronavirus which include multiple mutations.

The inventors herein have made vaccines against beta coronavirus which comprises a VLP. VLPs are multiprotein structures which are generally composed of one or more viral proteins. VLP's mimic the conformation of viruses but lack genetic material, and therefore are not infectious. They can form (or “self-assemble”) upon expression of a viral structural protein under appropriate circumstances. VLP vaccines overcome some of the disadvantages of more traditional vaccines prepared using attenuated viruses because they can be produced without the need to have any live virus present during the production process. A wide variety of VLPs have been prepared. For example, VLPs including single or multiple capsid proteins either with or without envelope proteins and/or surface glycoproteins have been prepared. In some cases, VLPs are non-enveloped and assemble by expression of just one major capsid protein. In other cases, VLPs are enveloped and can comprise multiple antigenic proteins found in the corresponding native virus. Self-assembly of enveloped VLPs is more complex than non-enveloped VLPs because of the complex reactions required for fusion with the host cell membrane (Garrone et al., 2011 Science Trans. Med. 3: 1-8) and “budding” of the VLP to form a fully enveloped separate particle. Accordingly, self-assembly of enveloped VLPs may not be successful and the formation and stability of enveloped VLP particles is difficult to predict. Formation of intact VLPs can be confirmed by imaging of the particles using electron microscopy.

VLPs typically resemble their corresponding native virus and can be multivalent particulate structures. The present disclosure encompasses the recognition that presentation of surface glycoproteins in the context of a VLP is advantageous for induction of neutralizing antibodies against such polypeptide as compared to other forms of antigen presentation, e.g., soluble antigens not associated with a VLP. Neutralizing antibodies most often recognize tertiary or quaternary structures; this often requires presenting antigenic proteins, like envelope glycoproteins, in their native viral conformation. VLP's present epitopes in a highly structured, repetitive array that enables efficient crosslinking of B cell receptors, leading to activation and expansion of high affinity B cells and subsequent antibody production (Bachmann, 1993). Indeed, VLP expression of a B cell antigen improved neutralizing titers by over 10-fold relative to immunization with the same amount of recombinant protein (Kirchmeier, 2014). Accordingly, use of VLPs as a vaccine modality may expand higher affinity B cell repertoires relative to recombinant protein or DNA/mRNA-based approaches, the latter approach being used in two widely used COVID-19 vaccines.

The VLPs of the invention comprise retroviral vectors. Retroviruses are enveloped RNA viruses that belong to the family Retroviridae. After infection of a host cell by a retrovirus, RNA is transcribed into DNA via the enzyme reverse transcriptase. DNA is then incorporated into the host cell's genome by an integrase enzyme and thereafter replicates as part of the host cell's DNA. The Retroviridae family includes the following genera Alpharetrovirus, Betaretrovirus, Gammearetrovirus, Deltaretrovirus, Epsilonretrovirus, Lentivirus and Spumavirus. The hosts for this family of retroviruses generally are vertebrates. Retroviruses produce an infectious virion containing a spherical nucleocapsid (the viral genome in complex with viral structural proteins) surrounded by a lipid bilayer derived from the host cell membrane.

Retroviral vectors can be used to generate VLPs that lack a retrovirus-derived genome and are therefore non-replicating. This is accomplished by replacement of most of the coding regions of the retrovirus with genes or nucleotide sequences to be transferred; so that the vector is incapable of making proteins required for additional rounds of replication. Depending on the properties of the glycoproteins present on the surface of the particles, VLPs have limited ability to bind to and enter the host cell but cannot propagate. Therefore, VLPs can be administered safely as an immunogenic composition (e.g., a vaccine).

The present invention utilizes VLPs comprising one or more retroviral structural proteins. In some embodiments, a structural protein for use in accordance with the present invention is Alpharetrovirus (e.g., Avian Leukosis Virus), Betaretrovirus (Mouse Mammary Tumor Virus), Gammearetrovirus (Murine Leukemia Virus), Deltaretrovirus (Bovine Leukemia Virus), Epsilonretrovirus (Walley Dermal Sarcoma Virus), Lentivirus (Human Immunodeficiency Virus 1) or Spumavirus (Chimpanzee Foamy Virus) structural protein. In certain embodiments, a structural polyprotein is a Murine Leukemia Virus (MLV) structural protein. In an embodiment of the invention the structural protein in a Moloney Murine Leukemia Virus (MMLV). Genomes of these retroviruses are readily available in databases.

In some embodiments, the retroviral structural protein for use in accordance with the present invention is a Gag polypeptide. The Gag proteins of retroviruses have an overall structural similarity and, within each group of retroviruses, are conserved at the amino acid level. Retroviral Gag proteins primarily function in viral assembly. Expression of Gag of some viruses (e.g., murine leukemia viruses, such as MMLV) in some host cells, can result in self-assembly of the expression product into VLPs. The Gag gene expression product in the form of a polyprotein gives rise to the core structural proteins of the VLP. Functionally, the Gag polyprotein is divided into three domains: the membrane binding domain, which targets the Gag polyprotein to the cellular membrane; the interaction domain which promotes Gag polymerization; and the late domain which facilitates release of nascent virions from the host cell. In general, the form of the Gag protein that mediates viral particle assembly is the polyprotein. Retroviruses assemble an immature capsid composed of the Gag polyprotein but devoid of other viral elements like viral protease with Gag as the structural protein of the immature virus particle.

A suitable Gag polypeptide for use in the invention is substantially homologous to a known retroviral Gag polypeptide. The MMLV-Gag gene encodes a 65 kDa polyprotein precursor which is proteolytically cleaved into 4 structural proteins (Matrix (MA); p12; Capsid (CA); and Nucleocapsid (NC)), by MLV protease, in the mature virion. In the absence of MLV protease, the polyprotein remains uncleaved and the resulting particle remains in an immature form. The morphology of the immature particle is different from that of the mature particle. In some embodiments of the invention, the Gag sequence does not include a gene encoding MLV protease. The gene encoding the MMLV nucleic acid is SEQ ID NO: 2. An exemplary codon optimized sequence of MMLV nucleic acid is provided as SEQ ID NO: 3.

Therefore, in some embodiments, a Gag polypeptide suitable for the present invention is substantially homologous to an MMLV-Gag polypeptide (SEQ ID NO:1). In some embodiments, a Gag polypeptide suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:1. In some embodiments, a Gag polypeptide suitable for the present invention is substantially identical to, or identical to SEQ ID NO: 1.

In some embodiments, a suitable MMLV-Gag polypeptide is encoded by a nucleic acid sequence having at least 80%, 85%, 90%, 91%, 92%, 93%, 94%, 95%, 96%, 97%, 98%, 99% sequence identity to SEQ ID NO:2. In some embodiments, a suitable MMLV-Gag polypeptide is encoded by a nucleic acid sequence having SEQ ID NO: 2 or a codon degenerate version thereof.

As is well known to those of skill in the art, it is possible to improve the expression of a nucleic acid sequence in a host organism by replacing the nucleic acids coding for a particular amino acid (i.e. a codon) with another codon which is better expressed in the host organism. One reason that this effect arises is due to the fact that different organisms show preferences for different codons. The process of altering a nucleic acid sequence to achieve better expression based on codon preference is called codon optimization. Various methods are known in the art to analyze codon use bias in various organisms and many computer algorithms have been developed to implement these analyses in the design of codon optimized gene sequences. Therefore, in some embodiments, a suitable MMLV-Gag polypeptide is encoded by a codon optimized version of a nucleic acid sequence encoding MMLV-Gag and having at least 80%, 85%, 90%, 91%, 92%, 93%, 94%, 95%, 96%, 97%, 98%, 99% sequence identity to SEQ ID NO:3. In some embodiments, a suitable MMLV-Gag polypeptide is encoded by a nucleic acid sequence which is substantially identical to, or identical to, SEQ ID NO: 3.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Examples of such programs are described in Altschul, et al., 1990, J. Mol. Biol., 215(3): 403-410; Altschul, et al., 1996, Methods in Enzymology 266:460-480; Altschul, et al., 1997 Nucleic Acids Res. 25:3389-3402; Baxevanis, et al., 1998, Bioinformatics: A Practical Guide to the Analysis of Genes and Proteins, Wiley; and Misener, et al., (eds.), 1999, Bioinformatics Methods and Protocols (Methods in Molecular Biology, Vol. 132), Humana Press. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Alternatively, the Gag polypeptide used in the invention may be a modified retroviral Gag polypeptide containing one or more amino acid substitutions, deletions, and/or insertions as compared to a wild-type or naturally-occurring Gag polypeptide while retaining substantial self-assembly activity. Typically, in nature, a Gag protein includes a large C-terminal extension which may contain retroviral protease, reverse transcriptase, and integrase enzymatic activity. Assembly of VLPs, however, generally does not require the presence of such components. In some cases, a retroviral Gag protein alone (e.g., lacking a C-terminal extension, lacking one or more of genomic RNA, reverse transcriptase, viral protease, or envelope protein) can self-assemble to form VLPs both in vitro and in vivo (Sharma S et al., 1997, Proc. Natl. Acad. Sci. USA 94: 10803-8).

The inventors of the present application have made VLPs which express beta coronavirus envelope glycoproteins on the surface which can cause an immune response in a subject. A humoral immune response is an immune response mediated by antibody molecules. Certain antibodies, called neutralizing antibodies, defend cells from infection by a virus and associated biological effects by recognizing and binding to a particular protein or antigen expressed by the virus. The envelope protein of coronaviruses are important targets for production of neutralizing antibodies. It is well known to those in the art that retroviral Gag-based enveloped VLPs can be used to express a variety of envelope glycoproteins for the purpose of eliciting neutralizing antibody responses. More specifically, evidence exists for expression of Class I viral fusion proteins such as HIV-1 gp120, metapneumovirus and Influenza HA, as well as Class III fusion proteins such as VSV G protein and CMV gB protein (Mammano et al., 1997, J. Virol. 71:3341-3345; Levy et al., 2013, Vaccine 31:2778-2785; Lemaitre et al., 2011, Clin. Microbiol. Infect. 1:732-737; Garrone et al, 2011; Kirchmeier et al., 2014, CVI 21: 174-180). However, there is little known about expression of coronavirus spike proteins, particularly with MLV-derived Gag. In U.S. Pat. No. 8,920,812, Example 1 describes a failure to express RSV F glycoprotein, a class II viral fusion protein, on the surface of a VLP produced using MLV Gag. The inventor hypothesized that the presence of the RSV F glycoprotein interfered with budding of the Gag viral particle through the cell membrane (see column 41, line 50). It was therefore not predictable that a retroviral Gag-based enveloped virus-like particle could be used to successfully express the coronavirus spike protein. Nevertheless, the present inventors have made several different embodiments of a beta coronavirus vaccine comprising one or more spike polypeptide antigens (e.g., from SARS CoV-2, SARS CoV and MERS-CoV) on the surface of a VLP. In some embodiments, the spike polypeptide antigens comprise modified polypeptides. In some embodiments, the spike polypeptide antigens have more than one genetic modification.

In some embodiments, a VLP of the invention includes a fusion protein of a spike polypeptide from a beta coronavirus (e.g., all or part of an extracellular portion of an beta coronavirus spike polypeptide) and a transmembrane and/or cytoplasmic domain that is not found in nature in the beta coronavirus protein (e.g., from another virus). In some embodiments, a fusion protein includes a spike polypeptide from a beta coronavirus (e.g., all or part of an extracellular portion of the spike polypeptide) and a transmembrane domain and/or cytoplasmic domain found in nature in the glycoprotein G from VSV which is referred to as VSV-G. The nucleotide and amino acid sequences of the VSV-G protein are known in the art.

The transmembrane domain of VSV-G can function to target the viral glycoprotein to the cell membrane (Compton T et al., 1989, Proc Natl Acad Sci USA 86:4112-4116). Swapping the transmembrane and cytoplasmic domains of VSV-G for the transmembrane and cytoplasmic domains of another protein has been used to increase the yield of the protein of interest in the VLP preparation and increase immunogenicity to neutralizing antibody response (Garrone et al., 2011). This modification was successful to increase yield and activity of a VLP expressing HCV-E1 protein (Garrone et al, 2011) and CMV-gB protein (Kirchmeier et al, 2014). However, this modification has also been associated with a significant loss of immunogenicity when used with certain viral antigens. In addition, expression of some glycoproteins has decreased after replacement of the transmembrane/cytoplasmic domain of the antigenic glycoprotein with the transmembrane/cytoplasmic domain from VSV. For example, loss of glycoprotein was reported in SARS virus (Broer et al., 2006, J. Vir. 80, 1302-1310). In RSV, a significant loss of immunogenicity was observed when the antigenic surface protein was modified to replace the transmembrane component with a sequence from VSV.

In some embodiments, an immunogenic composition of the present invention comprises a VLP comprising a wild type spike polypeptide from SARS-CoV-2, the sequence of which is SEQ ID NO: 4 or a codon degenerate version of SEQ ID NO: 4. A nucleic acid which encodes for the polypeptide is shown as SEQ ID NO: 5. A codon optimized version of SEQ ID NO: 5 is shown as SEQ ID NO: 6. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 4. In some embodiments, the present invention comprises an immunogenic composition comprising a VLP comprising a polypeptide having an amino acid sequence which is SEQ ID NO: 4 or a codon degenerate version of SEQ ID NO: 4. In some embodiments, the polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 5. In some embodiments, the polypeptide is encoded by a codon optimized version of the nucleic acid sequence of SEQ ID NO: 5, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 6. In some embodiments, the polypeptide is encoded by a nucleic acid sequence having SEQ ID NO: 6.

In some embodiments, an immunogenic composition of the present invention comprises a VLP comprising a modified spike polypeptide from SARS-CoV-2 which has been modified to replace the transmembrane and cytoplasmic segments with corresponding segments from VSV, the sequence of which is SEQ ID NO: 26 or a codon degenerate version of SEQ ID NO: 26. A nucleic acid which encodes for the polypeptide is shown as SEQ ID NO: 25. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 25. In some embodiments, the present invention comprises an immunogenic composition comprising a VLP comprising a polypeptide having an amino acid sequence which is SEQ ID NO: 25 or a codon degenerate version of SEQ ID NO: 25. In some embodiments, the polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 26. In some embodiments, the mutated polypeptide is encoded by a nucleic acid sequence having SEQ ID NO: 26. In some embodiments, the polypeptide is encoded by a codon optimized version of the nucleic acid sequence of SEQ ID NO: 26, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 27.

In some embodiments, an immunogenic composition of the present invention comprises a VLP comprising a wild type spike polypeptide from SARS-CoV, the sequence of which is SEQ ID NO: 7 or a codon degenerate version of SEQ ID NO: 7. A nucleic acid which encodes for the polypeptide is shown as SEQ ID NO: 8. A codon optimized version of SEQ ID NO: 8 is shown as SEQ ID NO: 9. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 7. In some embodiments, the present invention comprises an immunogenic composition comprising a VLP comprising a polypeptide having an amino acid sequence which is SEQ ID NO: 7 or a codon degenerate version of SEQ ID NO: 7. In some embodiments, the polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 8. In some embodiments, the polypeptide is encoded by a codon optimized version of the nucleic acid sequence of SEQ ID NO: 8, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 9. In some embodiments, the polypeptide is encoded by a nucleic acid sequence having SEQ ID NO: 9.

In some embodiments, an immunogenic composition of the present invention comprises a VLP comprising a wild type spike polypeptide from MERS-CoV, the sequence of which is SEQ ID NO: 10 or a codon degenerate version of SEQ ID NO: 10. A nucleic acid which encodes for the polypeptide is shown as SEQ ID NO: 11. A codon optimized version of SEQ ID NO:11 is shown as SEQ ID NO: 12. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 10. In some embodiments, the present invention comprises an immunogenic composition comprising a VLP comprising a polypeptide having an amino acid sequence which is SEQ ID NO: 10 or a codon degenerate version of SEQ ID NO: 10. In some embodiments, the polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 11. In some embodiments, the polypeptide is encoded by a codon optimized version of the nucleic acid sequence of SEQ ID NO: 11, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 12. In some embodiments, the polypeptide is encoded by a nucleic acid sequence having SEQ ID NO: 12.

In some embodiments, immunogenic compositions of the present invention comprise VLPs comprising variants of beta coronavirus spike glycoproteins. In some embodiments, a variant spike glycoprotein has been modified to delete the furin cleavage site from the spike polypeptide. In some embodiments, the spike glycoprotein has been modified to replace lysine (986) and valine (987) residues with proline residues. In some embodiments, the spike glycoprotein has been modified to delete the furin cleavage site and to replace lysine (986) and valine (987) residues with proline residues. Each such modification is further described below.

It is known that the coronavirus spike protein includes a site where the protease, furin, cleaves the S polypeptide into 51 and S2 subunits during the process of virion maturation. A modified spike protein construct was produced wherein the amino acid sequence was modified to remove the furin cleavage site, thus retaining the spike polypeptide in its immature form. It is possible that the furin-cleavage site mutated version of the spike protein, which does not undergo normal cleavage and maturation, will show enhanced cell receptor binding and cell entry, indicating that immunity against this structure may result in humoral immunity with greater neutralizing activity.

In some embodiments, an immunogenic composition of the invention comprises a VLP comprising a modified SARS-CoV-2 spike polypeptide with a mutated furin cleavage site as compared to a wild-type or naturally-occurring SARS-CoV-2 spike polypeptide. The sequence for an exemplary modified SARS-CoV-2 polypeptide is shown as SEQ ID NO: 16. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 16. In some embodiments, the present invention comprises an immunogenic composition comprising a VLP comprising a polypeptide having an amino acid sequence which is SEQ ID NO: 16 or a codon degenerate version of SEQ ID NO: 16. In some embodiments, the modified polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 17. In some embodiments, the modified polypeptide is encoded by a codon optimized version of the nucleic acid sequence of SEQ ID NO: 17, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 18. In some embodiments, the mutated polypeptide is encoded by a nucleic acid sequence having SEQ ID NO: 18.

It is known from previous studies of SARS-CoV and MERS-CoV that substitution of two amino acid residues with proline residues results in stabilisation of the S2 subunit in its prefusion conformation (Pallesen et al., 2017 PNAS. 114:35; Wrapp et al (2020) Science: 367: 1260-1263). Therefore, it is possible that such a mutation could enhance the immune response to coronavirus. Accordingly, a SARS-CoV-2 polypeptide construct was prepared which has been modified to replace lysine (986) and valine (987) residues with proline residues. In some embodiments, an immunogenic composition of the invention comprises a VLP comprising a SARS-CoV-2 polypeptide which has been modified to replace lysine (986) and valine (987) residues with proline residues as compared to a wild-type or naturally-occurring SARS-CoV-2 polypeptide. The sequence of an exemplary modified polypeptide is shown in SEQ ID NO: 13. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 13. In some embodiments, the present invention comprises an immunogenic composition comprising a VLP comprising a polypeptide having an amino acid sequence which is SEQ ID NO: 13 or a codon degenerate version of SEQ ID NO: 13. In some embodiments, the modified polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 14. In some embodiments, the modified polypeptide is encoded by a codon optimized version of the nucleic acid sequence of SEQ ID NO: 14, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO:15. In some embodiments, the modified polypeptide is encoded by a nucleic acid sequence having SEQ ID NO: 15.

In a further variation, a SARS-CoV-2 polypeptide construct was prepared which has been modified to replace lysine (986) and valine (987) residues with proline residues and which have been further modified to remove the furin cleavage site. In some embodiments, an immunogenic composition of the invention comprises a VLP comprising a SARS-CoV-2 polypeptide which has been modified to replace lysine (986) and valine (987) residues with proline residues and to remove the furin cleavage site as compared to a wild-type or naturally-occurring SARS-CoV-2 polypeptide. The sequence of an exemplary modified polypeptide is shown in SEQ ID NO: 19. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 19. In some embodiments, the present invention comprises an immunogenic composition comprising a VLP comprising a polypeptide having an amino acid sequence which is SEQ ID NO: 19 or a codon degenerate version of SEQ ID NO: 19. In some embodiments, the modified polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 20. In some embodiments, the modified polypeptide is encoded by a codon optimized version of the nucleic acid sequence of SEQ ID NO: 20, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO:21. In some embodiments, the modified polypeptide is encoded by a nucleic acid sequence having SEQ ID NO: 21.

In some embodiments, a VLP described herein comprises a fusion protein comprising an extracellular domain (or a portion thereof) of a coronavirus spike polypeptide, and a transmembrane domain and cytoplasmic tail from an envelope protein from VSV. In some embodiments, the immunogenic composition of the invention comprises a VLP comprising a coronavirus spike polypeptide modified to replace the transmembrane domain and cytoplasmic tail with the transmembrane domain and cytoplasmic tail from VSV. Any of the coronavirus spike proteins described herein may be modified to replace the transmembrane domain and cytoplasmic tail with a transmembrane domain and cytoplasmic tail from VSV.

In one particular embodiment, the inventors have constructed a SARS-CoV-2 spike protein which protein has been modified to replace the transmembrane domain and cytoplasmic tail with a transmembrane domain and cytoplasmic tail from VSV; to replace lysine (986) and valine (987) residues with proline residues; and to remove the furin cleavage site. This triple modified SARS-CoV-2 protein includes the double proline mutation directed to enhanced stability and a mutated furin cleavage site, which is associated with enhanced receptor binding. Further, it includes the transmembrane domain and cytoplasmic tail from VSV, which are associated with improved expression on the VLP envelope. The sequence of this triple modified coronavirus spike polypeptide is shown as SEQ ID NO: 22 (shown above with the portion from VSV in bold text at the end of the sequence). In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 22. In some embodiments, the modified polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 23. In some embodiments, the modified polypeptide is encoded by a codon optimized version of the nucleic acid sequence of SEQ ID NO: 23, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 23. In some embodiments, the modified polypeptide is encoded by a nucleic acid sequence having SEQ ID NO: 24.

In a further embodiment, the inventors have constructed a SARS-CoV-2 spike protein from the Beta variant which protein has been modified to replace the transmembrane domain and cytoplasmic tail with a transmembrane domain and cytoplasmic tail from VSV; to replace lysine (986) and valine (987) residues (based on the positions in the Wuhan reference) with proline residues; and to remove the furin cleavage site. This triple modified SARS-CoV-2 protein from the Beta variant includes the double proline mutation directed to enhanced stability and a mutated furin cleavage site, which is associated with enhanced receptor binding. Further, it includes the transmembrane domain and cytoplasmic tail from VSV, which are associated with improved expression on the VLP envelope. The sequence of this triple modified coronavirus spike polypeptide is shown as SEQ ID NO: 28. In some embodiments, the present invention comprises an immunogenic composition comprising a VLP comprising a polypeptide having an amino acid sequence which is at least 80%, 85%, 90%, 91%, 92%, 93%, 94%, 95%, 96%, 97%, 98%, 99% or more homologous to SEQ ID NO: 28. In some embodiments, the modified polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 29. In some embodiments, the modified polypeptide is encoded by a codon optimized version of the nucleic acid sequence of SEQ ID NO: 29, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 29. In some embodiments, the modified polypeptide is encoded by a nucleic acid sequence having SEQ ID NO: 30.

Mixtures of antigens can induce broad reactive immunity therefore, combinations of coronavirus antigens can be used to enhance the breadth of the immune response. VLPs can be used to express two (bivalent) or three (trivalent) viral antigens in their native conformation, thus inducing a potent B cell response. Previous studies using Zika viral epitopes demonstrated that the combination of two antigens on a single bivalent VLP generated a significantly more potent immune response than two monovalent VLPs expressing the same antigens (U.S. Pat. No. 8,920,812).

Accordingly, the VLPs of the present disclosure include bivalent VLPs containing two wild type coronavirus spike proteins, two modified coronavirus spike proteins described herein or any combination of the wild type and modified coronavirus spike proteins described herein. The VLPs of the present disclosure also include trivalent VLPs containing three wild type coronavirus spike proteins, three modified coronavirus spike proteins described herein or any combination of the wild type and modified coronavirus spike proteins described herein. One or more of any of the wild type or modified spike proteins expressed on a bivalent or a trivalent VLP may be further modified to replace the transmembrane domain and the cytoplasmic tail with the transmembrane domain and cytoplasmic tail from VSV.

In a preferred embodiment, the VLP of the present disclosure is a trivalent VLP comprising a spike protein from SARS-CoV-2, a spike protein from SARS-CoV and a spike protein from MERS-CoV. One or more of the spike proteins may be modified to replace the transmembrane domain and the cytoplasmic tail with the transmembrane domain and cytoplasmic tail from VSV.

In some embodiments, an immunogenic composition of the present invention comprises a trivalent VLP comprising a wild type spike polypeptide from SARS-CoV-2, the sequence of which is SEQ ID NO: 4 or a codon degenerate version of SEQ ID NO: 4; a spike polypeptide from SARS-CoV the sequence of which is SEQ ID NO: 7 or a codon degenerate version of SEQ ID NO: 7; and a spike polypeptide from MERS the sequence of which is SEQ ID NO: 10 or a codon degenerate version of SEQ ID NO: 10. A nucleic acid which encodes for the SARS-CoV-2 polypeptide is shown as SEQ ID NO: 5. A codon optimized version of SEQ ID NO: 5 is shown as SEQ ID NO: 6. A nucleic acid which encodes for the SARS-CoV polypeptide is shown as SEQ ID NO: 8. A codon optimized version of SEQ ID NO: 8 is shown as SEQ ID NO: 9. A nucleic acid which encodes for the MERS polypeptide is shown as SEQ ID NO: 11. A codon optimized version of SEQ ID NO:11 is shown as SEQ ID NO: 12. In some embodiments, the present invention comprises an immunogenic composition comprising a VLP comprising polypeptides having an amino acid sequence which is at least 80%, 85%, 90%, 91%, 92%, 93%, 94%, 95%, 96%, 97%, 98%, 99% or more homologous to SEQ ID NO: 4, 80%, 85%, 90%, 91%, 92%, 93%, 94%, 95%, 96%, 97%, 98%, 99% or more homologous to SEQ ID NO: 7 and 80%, 85%, 90%, 91%, 92%, 93%, 94%, 95%, 96%, 97%, 98%, 99% or more homologous to SEQ ID NO: 10. In some embodiments, the SARS-CoV-2 polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 5. In some embodiments, the polypeptide is encoded by a codon optimized version of the nucleic acid sequence of SEQ ID NO: 5, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 6. In some embodiments, the polypeptide is encoded by a nucleic acid sequence having SEQ ID NO: 6. In some embodiments, the SAR-CoV polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 8. In some embodiments, the polypeptide is encoded by a codon optimized version of the nucleic acid sequence of SEQ ID NO: 8, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 9. In some embodiments, the polypeptide is encoded by a nucleic acid sequence having SEQ ID NO: 9. In some embodiments, the MERS polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 11. In some embodiments, the polypeptide is encoded by a codon optimized version of the nucleic acid sequence of SEQ ID NO: 11, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 12. In some embodiments, the polypeptide is encoded by a nucleic acid sequence having SEQ ID NO: 12.

In some embodiments, an immunogenic composition of the present invention comprises a trivalent VLP comprising a modified spike polypeptide from SARS-CoV-2, the sequence of which is SEQ ID NO: 22 or a codon degenerate version of SEQ ID NO: 22; a spike polypeptide from SARS-CoV the sequence of which is SEQ ID NO: 7 or a codon degenerate version of SEQ ID NO: 7; and a spike polypeptide from MERS the sequence of which is SEQ ID NO: 10 or a codon degenerate version of SEQ ID NO: 10. A nucleic acid which encodes for the SARS-CoV-2 polypeptide is shown as SEQ ID NO: 5. A codon optimized version of SEQ ID NO: 5 is shown as SEQ ID NO: 6. A nucleic acid which encodes for the SARS-CoV polypeptide is shown as SEQ ID NO: 8. A codon optimized version of SEQ ID NO: 8 is shown as SEQ ID NO: 9. A nucleic acid which encodes for the MERS polypeptide is shown as SEQ ID NO: 11. A codon optimized version of SEQ ID NO:11 is shown as SEQ ID NO: 12. In some embodiments, the present invention comprises an immunogenic composition comprising a VLP comprising polypeptides having an amino acid sequence which is at least 80%, 85%, 90%, 91%, 92%, 93%, 94%, 95%, 96%, 97%, 98%, 99% or more homologous to SEQ ID NO: 22, 80%, 85%, 90%, 91%, 92%, 93%, 94%, 95%, 96%, 97%, 98%, 99% or more homologous to SEQ ID NO: 7 and 80%, 85%, 90%, 91%, 92%, 93%, 94%, 95%, 96%, 97%, 98%, 99% or more homologous to SEQ ID NO: 10. In some embodiments, the SARS-CoV-2 polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 23. In some embodiments, the polypeptide is encoded by a codon optimized version of the nucleic acid sequence of SEQ ID NO: 23, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 24. In some embodiments, the polypeptide is encoded by a nucleic acid sequence having SEQ ID NO: 24. In some embodiments, the SAR-CoV polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 8. In some embodiments, the polypeptide is encoded by a codon optimized version of the nucleic acid sequence of SEQ ID NO: 8, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 9. In some embodiments, the polypeptide is encoded by a nucleic acid sequence having SEQ ID NO: 9. In some embodiments, the MERS polypeptide is encoded by a nucleic acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 11. In some embodiments, the polypeptide is encoded by a codon optimized version of the nucleic acid sequence of SEQ ID NO: 11, which is at least 50%, 55%, 60%, 65%, 70%, 75%, 80%, 85%, 90%, 91%, 92%, 93%, 94%, 95%, 96%, 97%, 98%, 99% or more homologous to the SEQ ID NO: 12. In some embodiments, the polypeptide is encoded by a nucleic acid sequence having SEQ ID NO: 12.

As can be seen in the Examples, the VLPs of the invention were able to elicit a strong immune response to SARS-CoV-2. In particular, each of the modified spike variants described herein was effective to induce a strong immune response (see Example 5). The trivalent VLPs of the invention (see Example 6) induced an antibody response against SARS-Cov-2, SARS-CoV and MERS. Moreover, immunization with the trivalent VLPs of the invention induced antibodies that recognized a related seasonal human coronavirus, 0C43, not included within the vaccine, demonstrating that the trivalent VLP has an ability to broaden immunity against coronaviruses. Unexpectedly, relative to immunization with a monovalent VLP, trivalent VLPs enriched the induction of antibodies with functional, neutralizing activity. This enrichment of neutralizing antibodies in shown in Table 8, which shows the ratio of endpoint neutralizing antibody titer to the endpoint antibody binding titer, using sera obtained from vaccinated mice.

The monovalent VLP which expresses the triple modified SARS-CoV-2 spike protein provides significant protection against infection by SARS-CoV-2 as demonstrated by a challenge study in golden hamster (Example 7). As shown in Example 7, the hamsters which were vaccinated with the VLP had significantly lower levels of viral RNA and improved clinical presentation as shown by body weight. Furthermore, the immunized hamsters were able to mount a stronger cytokine response than the unvaccinated hamsters. Interim data from a Phase 1a clinical trial in humans using a vaccine formulation comprising the triple modified SARS-CoV-2 spike protein adjuvanted with aluminum phosphate adjuvant showed that all subjects receiving two doses had seroconverted 56 days after the second dose (see Example 11).

The VLPs of the invention have demonstrated a broad immune response that is effective against a variant of SARS-CoV-2. As described in Example 9, a trivalent VLP expressing the triple modified SARS-CoV-2 spike protein, a native MERS spike protein and a native SARS-CoV protein and a monovalent VLP expressing the triple modified SARS-CoV-2 spike protein were evaluated for their ability to induce antibodies against the Beta (South Africa) variant of SARS-CoV-2 in mice. Surprisingly, both the trivalent and the monovalent constructs elicited antibodies to the Beta variant. Even more surprising was the fact that the antibody titres were similar for the Beta strain and the ancestral L strain of SARS-CoV-2. Accordingly, both the trivalent and the monovalent VLPs expressing the triple modified SARS-CoV-2 spike protein were unexpectedly effective at inducing a potent antibody response to a SARS-CoV-2 variant which has demonstrated significant escape from other COVID-19 vaccines.

Enhanced antibody binding and neutralizing activity was observed when a monovalent VLP which expresses a triple modified SARS-CoV-2 spike protein based on the Beta variant (SEQ ID NO: 30) was used as a booster vaccine following a first vaccination with a vaccine based on a triple modified SARS-CoV-2 spike protein based on the ancestral strain (SEQ ID NO: 24). This heterologous boosting strategy also resulted in enhanced antibody binding against the Delta variant indicating that the combination strategy enhances the breadth of the humoral immune response (see Example 12).

The VLPs of the invention also had an effect on the nature of the antibody response. As shown in Example 10, mice immunized with a monovalent VLP of the invention expressing wild type SARS-CoV-2 spike protein produced a higher amount of IgG2b than those immunized with a recombinant spike protein. Higher IgG2b is associated with a TH1 immune response and may result in a higher level of cell-mediated immunity. Therefore, presentation of the spike protein on an VLP resulted in a response correlated to cell-mediated immunity.

It will be appreciated that a composition comprising VLPs will typically include a mixture of VLPs with a range of sizes. It is to be understood that the diameter values listed below correspond to the most frequent diameter within the mixture. In some embodiments >90% of the vesicles in a composition will have a diameter which lies within 50% of the most frequent value (e.g., 1000±500 nm). In some embodiments, the distribution may be narrower, e.g., >90% of the vesicles in a composition may have a diameter which lies within 40, 30, 20, 10 or 5% of the most frequent value. In some embodiments, sonication or ultra-sonication may be used to facilitate VLP formation and/or to alter VLP size. In some embodiments, filtration, dialysis and/or centrifugation may be used to adjust the VLP size distribution.

In general, VLPs produced in accordance with the methods of the present disclosure may be of any size. In certain embodiments, the composition may include VLPs with diameters in the range of about 20 nm to about 300 nm. In some embodiments, a VLP is characterized in that it has a diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs within a population show an average diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs in a population have a polydispersity index that is less than 0.5 (e.g., less than 0.45, less than 0.4, or less than 0.3). In some embodiments, VLP diameter is determined by nanosizing. In some embodiments, VLP diameter is determined by electron microscopy.

VLPs in accordance with the present invention may be prepared according to general methodologies known to the skilled person. For example, nucleic acid molecules, reconstituted vectors or plasmids may be prepared using sequences which are known in the art. Such sequences are available from banks, and material may be obtained from various collections, published plasmids, etc. These elements can be isolated and manipulated using techniques well known to the skilled artisan, or available in the art. Various synthetic or artificial sequences may also be produced from computer analysis or through (high throughput) screening of libraries. Recombinant expression of the polypeptides for VLPs requires construction of an expression vector containing a polynucleotide that encodes one or more polypeptide(s). Once a polynucleotide encoding one or more polypeptides has been obtained, the vector for production of the polypeptide may be produced by recombinant DNA technology using techniques known in the art. Expression vectors that may be utilized in accordance with the present invention include, but are not limited to mammalian and avian expression vectors, bacculovirus expression vectors, plant expression vectors (e.g., Cauliflower Mosaic Virus (CaMV), Tobacco Mosaic Virus (TMV)), plasmid expression vectors (e.g., Ti plasmid), among others.

The VLPs of the invention may be produced in any available protein expression system. Typically, the expression vector is transferred to a host cell by conventional techniques and the transfected cells are then cultured by conventional techniques to produce VLPs. In some embodiments, VLPs are produced using transient transfection of cells. In some embodiments, VLPs are produced using stably transfected cells. Typical cell lines that may be utilized for VLP production include, but are not limited to, mammalian cell lines such as human embryonic kidney (HEK) 293, WI 38, Chinese hamster ovary (CHO), monkey kidney (COS), HT1080, C10, HeLa, baby hamster kidney (BHK), 3T3, C127, CV-1, HaK, NS/O, and L-929 cells. Specific non-limiting examples include, but are not limited to, BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 cells subcloned for growth in suspension culture, Graham et al., J. Gen Virol., 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, cell lines that may be utilized for VLP production include insect (e.g., Sf-9, Sf-21, Tn-368, Hi5) or plant (e.g., Leguminosa, cereal, or tobacco) cells. It will be appreciated in some embodiments, particularly when glycosylation is important for protein function, mammalian cells are preferable for protein expression and/or VLP production (see, e.g., Roldao A et al., 2010 Expt Rev Vaccines 9:1149-76).

It will be appreciated that a cell strain may be chosen which modulates the expression of the inserted sequences, or modifies and processes the gene product in a specific way. Such modifications (e.g., glycosylation) and processing (e.g., cleavage or transport to the membrane) of protein products may be important for generation of a VLP or function of a VLP polypeptide or additional polypeptide (e.g., an adjuvant or additional antigen). Different cells have characteristic and specific mechanisms for post-translational processing and modification of proteins and gene products. Appropriate cell lines or host systems can be chosen to ensure the correct modification and processing of the foreign protein expressed. Generally, eukaryotic host cells (also referred to as packaging cells (e.g., 293T human embryo kidney cells)) which possess appropriate cellular machinery for proper processing of the primary transcript, glycosylation and phosphorylation of the gene product may be used in accordance with the present invention.

VLPs may be purified according to known techniques, such as centrifugation, gradients, sucrose-gradient ultracentrifugation, tangential flow filtration and chromatography (e.g., ion exchange (anion and cation), affinity and sizing column chromatography), or differential solubility, among others. Alternatively, or additionally, cell supernatant may be used directly, with no purification step. Additional entities, such as additional antigens or adjuvants may be added to purified VLPs.

In accordance with the present invention, cells may be transfected with a single expression vector. In some embodiments, a single expression vector encodes more than one element of a VLP (e.g., more than one of structural polyprotein, coronavirus spike protein, etc.). For example, in some embodiments, a single expression vector encodes two or more elements of a VLP. In some embodiments, a single expression vector encodes three of more elements of a VLP. In an embodiment of the invention, a single expression vector encodes a Gag polypeptide and a coronavirus spike glycoprotein.

In some embodiments, cells are transfected with two or more expression vectors. For example, in some embodiments, cells are transfected with a first vector encoding a Gag polypeptide and a second vector encoding a coronavirus spike glycoprotein and “monovalent” VLPs comprising a coronavirus spike glycoprotein are produced. In some embodiments, cells are transfected with a first vector encoding a Gag polypeptide, a second vector encoding a coronavirus spike glycoprotein and a third vector encoding another coronavirus spike glycoprotein. In such embodiments, “bivalent” VLPs comprising two coronavirus spike glycoproteins are produced. In some embodiments, cells are transfected with a first vector encoding a Gag polypeptide, a second vector encoding a coronavirus spike glycoprotein, and a third vector encoding two coronavirus spike glycoproteins. In such embodiments, “trivalent” VLPs comprising three coronavirus spike glycoproteins are produced.

As further demonstrated in the Examples, modification of the SARS-CoV-2 spike protein had a significant effect on the yield of the VLPs. Referring to Table 1, in Example 3, the VLP expressing the triple modified SARS-CoV-2 spike protein (Group 3) showed significantly higher spike protein yield that other monovalent VLPs expressing SARS-CoV-2 spike proteins. Accordingly, this embodiment of the VLP can be manufactured in higher volumes, which is important for addressing demand in pandemic situations.

In some embodiments, monovalent, bivalent, or trivalent VLPs are admixed. For example, in some embodiments, monovalent and bivalent VLPs are admixed to form a trivalent VLP mixture. In some embodiments two monovalent VLPs are admixed to form a bivalent VLP mixture.

The present invention provides pharmaceutical compositions comprising the VLPs described herein and, optionally, further comprising the glycoproteins and glycoprotein variants described herein. In some embodiments, the present invention provides a VLP and at least one pharmaceutically acceptable excipient, adjuvant and/or carrier. Such pharmaceutical compositions may optionally comprise and/or be administered in combination with one or more additional therapeutically active substances. The provided pharmaceutical compositions are useful as prophylactic agents (i.e., vaccines) in the prevention of SARS, MERS or COVID-19 infection or of negative ramifications associated or correlated with SARS, MERS or COVID-19 infection. The provided pharmaceutical compositions are also useful as prophylactic agents against certain variants of SARS-CoV-2. In some embodiments, pharmaceutical compositions are formulated for administration to humans.

Pharmaceutical compositions provided here may be provided in a sterile injectable form (e.g., a form that is suitable for subcutaneous injection or intravenous infusion). For example, in some embodiments, pharmaceutical compositions are provided in a liquid dosage form that is suitable for injection. In some embodiments, pharmaceutical compositions are provided as powders (e.g. lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). Suitable excipients include, for example, water, saline, dextrose, sucrose, trehalose, glycerol, ethanol, or similar, and combinations thereof. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. In some embodiments, pharmaceutical compositions comprise one or more preservatives. In some embodiments, pharmaceutical compositions comprise no preservative.

In some embodiments, a pharmaceutical composition is sufficiently immunogenic as a vaccine (e.g., in the absence of an adjuvant). In some embodiments, immunogenicity of a pharmaceutical composition is enhanced by including an adjuvant. Any adjuvant may be used in accordance with the present invention. A large number of adjuvants are known; a useful compendium of many such compounds is prepared by the National Institutes of Health and can be found (www.niaid.nih.gov/daids/vaccine/pdf/compendium.pdf). See also Allison, 1998, Dev. Biol. Stand., 92:3-11, Unkeless et al., 1998, Annu. Rev. Immunol., 6:251-281, and Phillips et al., 1992, Vaccine, 10:151-158. Hundreds of different adjuvants are known in the art and may be employed in the practice of the present invention. Exemplary adjuvants that can be utilized in accordance with the invention include, but are not limited to, cytokines, gel-type adjuvants (e.g., aluminum hydroxide, aluminum phosphate, calcium phosphate, etc.), microbial adjuvants (e.g., immunomodulatory DNA sequences that include CpG motifs; endotoxins such as monophosphoryl lipid A; exotoxins such as cholera toxin, E. coli heat labile toxin, and pertussis toxin; muramyl dipeptide, etc.), oil-emulsion and emulsifier-based adjuvants (e.g., Freund's Adjuvant, MF59 [Novartis], SAF, etc.), particulate adjuvants (e.g., liposomes, biodegradable microspheres, saponins, etc.), synthetic adjuvants (e.g., nonionic block copolymers, muramyl peptide analogues, polyphosphazene, synthetic polynucleotides, etc.) and/or combinations thereof. Other exemplary adjuvants include some polymers (e.g., polyphosphazenes; described in U.S. Pat. No. 5,500,161, Q57, Q S21, squalene, tetrachlorodecaoxide, etc.

In one embodiment, a pharmaceutical composition comprises a VLP of the invention formulated with aluminum phosphate adjuvant. Referring to Example 11, a triple modified SARS-CoV-2 spike protein (SEQ ID: 24) formulated with aluminum phosphate adjuvant (0.33 mg/ml) in a drug substance referred to as VBI-2902a was administered to human subjects in a Phase 1a clinical study. Interim data taken at day 56 showed that all subjects receiving two doses seroconverted after the second dose.

In some embodiments, pharmaceutical compositions are provided in a form that can be refrigerated and/or frozen. In some embodiments, pharmaceutical compositions are provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of VLP formulations for longer than the specified time results in VLP degradation.

A pharmaceutical composition in accordance with the invention may be prepared, packaged, and/or sold as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject and/or depending upon the route by which the composition is to be administered. By way of example, the composition may comprise between 0.1% and 100% (w/w) active ingredient.

Provided compositions and methods of the present disclosure are useful for prophylaxis and/or treatment of SARS, MERS or COVID-19 infection in a subject, including human adults and children. In general however they may be used with any animal. If desired, the methods herein may also be used with farm animals, such as ovine, avian, bovine, porcine and equine breeds. For the purposes of the present disclosure, vaccination can be administered before, during, and/or after exposure to a disease-causing agent, and in certain embodiments, before, during, and/or shortly after exposure to the agent. In some embodiments, vaccination includes multiple administrations, appropriately spaced in time, of a vaccinating composition.

Compositions described herein will generally be administered in such amounts and for such a time as is necessary or sufficient to induce an immune response. Dosing regimens may consist of a single dose or a plurality of doses over a period of time. The exact amount of an immunogenic composition (e.g., VLP) to be administered may vary from subject to subject and may depend on several factors. Thus, it will be appreciated that, in general, the precise dose used will depend not only on the weight of the subject and the route of administration, but also on the age of the subject. In certain embodiments a particular amount of a VLP composition is administered as a single dose. In certain embodiments, a particular amount of a VLP composition is administered as more than one dose (e.g., 1-3 doses that are separated by 1-12 months).

In some embodiments, a provided composition is administered in an initial dose and in at least one booster dose. In some embodiments, a provided composition is administered in an initial dose and two, three or four booster doses. In some embodiments, a provided composition is administered in an initial dose and in at least one booster dose about one month, about two months, about three months, about four months, about five months, or about six months following the initial dose. In some embodiments, a provided composition is administered in a second booster dose about six months, about seven months, about eight months, about nine months, about ten months, about eleven months, or about one year following the initial dose. In some embodiments, a provided composition is administered in a booster dose every 1 year, 2 years, 3 years, 4 years, 5 years, 6 years, 7 years, 8 years, 9 years, or 10 years.

In a preferred embodiment, a composition comprising a VLP which expresses a triple modified SARS-CoV-2 spike protein based on the Beta variant (SEQ ID NO: 30) is used as a booster vaccine following a first vaccination with a composition comprising a triple modified SARS-CoV-2 spike protein based on the ancestral strain of SARS-CoV-2 (SEQ ID NO: 24).

In certain embodiments, provided compositions may be formulated for delivery parenterally, e.g., by injection. In such embodiments, administration may be, for example, intravenous, intramuscular, intradermal, or subcutaneous, or via by infusion or needleless injection techniques. In certain embodiments, the compositions may be formulated for peroral delivery, oral delivery, intranasal delivery, buccal delivery, sublingual delivery, transdermal delivery, transcutaneous delivery, intraperitoneal delivery, intravaginal delivery, rectal delivery or intracranial delivery.

In some embodiments, upon administration to a subject, provided VLPs induce a humoral immune response in the subject. In some embodiments, the humoral immune response in a subject is sustained for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, at least about 24 months, at least about 28 months, at least about 32 months, at least about 36 months, at least about 40 months, at least about 44 months, at least about 48 months, or longer.

In some embodiments, upon administration to a subject, provided VLPs induce a cellular immune response in the subject. In some embodiments, the cellular immune response in a subject is sustained for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least 12 months.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some exemplary modes of making and practicing certain compositions that are described herein. It should be understood that these examples are for illustrative purposes only and are not meant to limit the scope of the compositions and methods described herein.

### Example 1: Construction of DNA Expression Plasmids

This Example describes development of expression plasmids and constructs for expression of recombinant coronavirus spike gene sequences. A standard expression plasmid generally consists of a promoter sequence of mammalian origin, an intron sequence, a PolyAdenylation signal sequence (PolyA), a pUC origin of replication sequence (pUC—pBR322 is a colE1 origin/site of replication initiation and is used to replicate plasmid in bacteria such as E. Coli (DH5α)), and an antibiotic resistance gene as a selectable marker for plasmid plaque selection. Within the plasmid following the intron are a variety of restriction enzyme sites that can be used to splice in a gene or partial gene sequence of interest.

The Propol II expression plasmid contains the pHCMV (early promoter for HCMV), a Beta-Globin Intron (BGL Intron), a rabbit Globin polyAdenylation signal sequence (PolyA), a pUC origin of replication sequence (pUC—pBR322 is a colE1 origin/site of replication initiation and is used to replicate plasmid in bacteria such as E. coli (DH5α)), and an ampicillin resistance gene β-lactamase (Amp R—selectable marker for plasmid confers resistance to ampicillin (100 μg/ml).

To develop a Gag MMLV expression construct (“MLV-Gag”), a complementary DNA (cDNA) sequence encoding a Gag polyprotein of MMLV (Gag without its C terminus Pol sequence) (SEQ ID NO: 3) was cloned in a Propol II expression vector. To develop all of coronavirus expression constructs, each of the following sequences:
i) SARS-CoV-2 (SEQ ID NO: 6);
ii) SARS-CoV (SEQ ID NO: 9);
iii) MERS (SEQ ID NO: 12);
iv) SARS-CoV-2 Proline Modified Spike Glycoprotein (SEQ ID NO: 15);
v) SARS-CoV-2 Furin Cleavage Modified (SEQ ID NO: 18);
vi) SARS-CoV-2 Proline and Furin Cleavage Modified (SEQ ID NO: 21);
vii) SARS-CoV-2 Proline and Furin Cleavage Modified with TM/Cyt from VSV (SEQ ID NO: 24); and
viii) SARS-CoV-2 modified with TM/Cyt from VSV (SEQ ID NO: 26)
   was cloned in a Propol II expression vector. The SARS-CoV-2 sequence was from the L strain of the virus that was originally identified in Wuhan China.

DNA plasmids were amplified in competent E. coli (DH5a) and purified with endotoxin-free preparation kits according to standard protocols.

### Example 2: Production of Virus-Like Particles

This Example describes methods for production of virus-like particles containing various recombinant coronavirus spike antigens described in Example 1.

293 SF-3F6 cell line derived from HEK 293 cells are a proprietary suspension cell culture grown in serum-free chemically defined media (CA 2,252,972 and U.S. Pat. No. 6,210,922). HEK 293 SF-3F6 cells were scaled up in shaker flasks at 37° C., 5% CO₂ at a speed of 80 rpm and subsequently seeded in a bioreactor using HyQSF4 Transfx293 media supplemented with L-glutamine (GE Bioscience) to obtain a target cell density of 0.9 to 1.2 million cells/ml and high viability (>90%). The cells were co-transfected at cell density of about ˜1 million cells/ml with different ratios of plasmids encoding coronavirus envelope polypeptides, plasmids encoding Gag and using high quality polyethyleneimine (PEIpro™) as transfection agent. The DNA plasmids and transfection agent were prepared in OptiPRO SFM medium (GE Biosciences). The bioreactor was monitored daily (˜24 hrs and 48 hrs post transfection) and cell density, viability and cell diameters recorded. The production broth was harvested at 48 hrs post transfection.

Total protein was determined on an aliquot by a Bradford assay quantification kit (BioRad). The Bradford Protein assay is based on the observation that the absorbance maximum for an acidic solution of Coomassie Brilliant Blue G-250 shifts from 465 nm to 595 nm when binding to protein occurs. Both hydrophobic and ionic interactions stabilize the anionic form of the dye, causing a visible color change. A spectrophotometer was used to measure the absorbance of the sample and Bradford Protein Reagent dye at 595 nm.

### Example 3: Production of Monovalent Vaccine Candidates

Four different monovalent virus like particles were produced using the method described in Example 2. The virus like particles were transfected with one of the four following SARS-CoV-2 nucleotide sequences:
1. Native form of SARS-CoV-2 (SEQ ID NO: 6);
2. SARS-CoV-2 Proline and Furin Cleavage Modified (SEQ ID NO: 21);
3. SARS-CoV-2 Proline and Furin Cleavage Modified with TM/Cyt from VSV (SEQ ID NO: 24); or
4. SARS-CoV-2 modified with TM/Cyt from VSV (SEQ ID NO: 26).

The total antigen content of the resulting products was measured and the results are shown in Table 1.

TABLE 1
Monovalent SARS-CoV-2 Virus Like Particle Yields

| | | | SARS-CoV-2 |
|---|---|---|---|
| | | Gag | spike protein |
| Group | Monovalent VLP Description | (μg) | (μg) |
| 1 | Native SARS-Cov-2 (SEQ ID #6) | 23,157 | 16.2 |
| 2 | SARS-CoV-2 Proline and Furin | 32,220 | 229.9 |
| | Cleavage Modified (SEQ ID NO: 21) | | |
| 3 | SARS-CoV-2 Proline and Furin | 22,916 | 639.2 |
| | Cleavage Modified with TM/Cyt | | |
| | from VSV (SEQ ID NO: 24) | | |
| 4 | SARS-CoV-2 modified with TM/Cyt | 19,332 | 49.95 |
| | from VSV (SEQ ID NO: 26) | | |

As can be seen from the data in Table 1, a significantly higher yield was obtained using Group 3, the SARS-CoV-2 sequence which had been modified by replacing the cytoplasmic and transmembrane segments with the corresponding segments form VSV.

### Example 4: Production of Trivalent Vaccine Candidates

Four different trivalent virus like particles were produced using the method described in Example 2. Each particle was transfected with plasmids encoding Gag, an antigenic sequence from MERS (SEQ ID NO: 12), an antigenic sequence from SARS-CoV (SEQ ID NO: 9) and one of the two following SARS-CoV-2 sequences:
1. Native form of SARS-CoV-2 envelope polypeptide (SEQ ID NO: 6); or
2. SARS-CoV-2 Proline and Furin Cleavage Modified with TM/Cyt from VSV (SEQ ID NO: 24).

The antigen content of the resulting products was measured and the results are shown in Table 2.

TABLE 2
Trivalent Coronavirus Virus Like Particle Yields

| | | | SARS-CoV-2 |
|---|---|---|---|
| | Trivalent VLP - SARS- | Gag | spike protein |
| Group | CoV-2 Spike Protein | (μg) | (μg) |
| 1 | Native SARS-Cov-2 | 20,358 | 28.8 |
| | (SEQ ID #6) | | |
| 2 | SARS-CoV-2 Proline | 17,382 | 404.4 |
| | and Furin Cleavage | | |
| | Modified with TM/Cyt | | |
| | from VSV (SEQ ID | | |
| | NO: 24) | | |

As can be seen from the data in Table 2, a significantly higher yield of trivalent VLPs was obtained using Group 2, the SARS-CoV-2 sequence with a stabilized prefusion form of the spike protein which was further modified with the TM/Cyt from VSV G protein (SEQ ID NO: 24).

### Example 5: Evaluation of the Potency of Monovalent SARS-CoV-2 VLP Vaccine Constructs

Naïve 6-8 week-old C57/BL6 mice (n=10) were immunized twice with approximately 1/20^{th} to 1/50^{th} the human dose of the SARS-CoV-2 VLP vaccine formulations shown below in Table 3. Immunization took place at day 0 and day 21. Animals were sacrificed 14 days after immunization and their serum was collected for subsequent analysis of anti-spike protein antibody titers, and neutralizing antibodies.

The SARS-CoV-2 VLPs were formulated with aluminum phosphate adjuvant (Adjuphos®) as shown in Table 3.

TABLE 3
Monovalent SARS-CoV-2 VLP Vaccine Formulations

| | | | | | Dose |
|---|---|---|---|---|---|
| | | μg SARS- | | | volume/ |
| | | CoV-2 | μg | Al+++ | Animal |
| Group | Monovalent SARS-CoV-2 | Spike/dose | Gag/dose | μg/dose | (μL) |
| 1 | SARS-CoV-2 native Spike protein VLP | 0.07 | 100.1 | 125 | 500 |
| 2 | SARS-CoV-2 Proline and Furin | 0.14 | 19.6 | 125 | 500 |
| 3 | Cleavage Modified (SEQ ID NO: 21) | 0.7 | 98.1 | 125 | 500 |
| 4 | SARS-CoV-2 Proline and Furin | 0.14 | 5.0 | 125 | 500 |
| 5 | Cleavage Modified with TM/Cyt | 0.7 | 25.05 | 125 | 500 |
| | from VSV (SEQ ID NO: 24) | | | | |
| 6 | SARS-CoV-2 modified with | 0.07 | 27.1 | 125 | 500 |
| 7 | TM/Cyt from VSV (SEQ ID NO: 26) | 0.14 | 54.2 | 125 | 500 |

Anti-Spike SARS-CoV-2 antibody titers were measured as follows: 96 well plates were coated overnight at 4° C., with SARS-COV-2 Spike Protein (51+S2) (Sinobiological, Cat #40589-V08B1) (0.1 μg/ml in DPBS). The following day, plates were blocked with 5% milk in ELISA wash buffer, for 1 hour at 37° C. Plates were washed with wash buffer, followed by addition of 2 fold dilutions of individual mouse sera starting at 1:10,000 to 1:1,200,000. Plates were incubated for 1.5 hours at 37° C., followed by plate washing and addition of Secondary Antibody: Goat anti-Mouse IgG1 (Bethyl, Cat #A90-131P), diluted 1:5,000 in 1% milk in ELISA wash buffer. Plates were incubated for 1 hour at 37° C. Plates were added with TMB One component Microwell substrate, incubated at room temperature for 10 minutes and then added with Stop solution. Absorbance was read at 450 nm using a MAXline plate reader. Results are shown below in Table 4.

The anti-spike total IgG binding titers reported in Table 4 represent the highest dilution of sera that still had an optical density of 0.1 or greater by ELISA measurement against recombinant SARS-CoV-2 spike protein. Unexpectedly, immunization of mice with just a single dose of VLPs expressing the stabilized prefusion form of the SARS-CoV-2 spike protein further modified with the TM/Cyt from the VSV-G protein (Group 4) induced antibody responses which were dramatically stronger than immunization of mice with VLPs expressing similar doses of SARS-CoV-2 spike protein but in different presentations (Groups 2, 7).

The antibody titers from the mice 14 days after each vaccination are shown in Table 4. P1 and P2 refer to the first and second vaccination. Results were pooled among individual animals.

TABLE 4
Monovalent SARS-CoV-2 VLP Vaccine Antibody Titres

| | | Anti- SARS-CoV-2 Spike |
|---|---|---|
| Group | Vaccination | Total IgG Binding Titers |
| Pooled Group 1 | P1Vd14 | 9,099 |
| | P2Vd14 | 310,103 |
| Pooled Group 2 | P1Vd14 | 74,612 |
| | P2Vd14 | 424,883 |
| Pooled Group 3 | P1Vd14 | 262,689 |
| | P2Vd14 | 321,427 |
| Pooled Group 4 | P1Vd14 | 341,493 |
| | P2Vd14 | 670,735 |
| Pooled Group 5 | P1Vd14 | 619,766 |
| | P2Vd14 | 359,528 |
| Pooled Group 6 | P1Vd14 | 1,108 |
| | P2Vd14 | 302,500 |
| Pooled Group 7 | P1Vd14 | 4,093 |
| | P2Vd14 | 221,990 |

As is shown in Table 4, each of the monovalent VLP vaccine formulations induced a strong antibody response in mice. In almost all formulations, the response was strongly enhanced by a second vaccination. One group, group 5 consisting of a vaccine formulation based on SARS-CoV-2 Proline and Furin Cleavage Modified with TM/Cyt from VSV (SEQ ID NO: 24), showed a reduced response after second vaccination. However, the response was very high after first vaccination, raising the possibility that the second vaccination exhausted the immune response in mice. It is possible that this response may not be seen in larger mammals such as humans.

Neutralizing antibodies were tested as follows. A constant amount of virus consisting of 100 plaque forming units (pfu) of a Canadian isolate of SARS-CoV-2 virus was mixed with 2-fold dilutions of the mouse serum specimens being tested, the dilutions ranging from 40 to 5120 times, followed by plating of the mixture onto cells of an appropriate cell line for the individual virus. The concentration of plaque forming units is determined by the number of plaques formed after a few days. A vital dye (e.g. crystal violet or neutral red) was then added for visualization of the plaques and the number of plaques in an individual plate with test serum was divided by the number of plaques present in a negative control sera to calculate the percentage neutralization. The plaque forming units were measured by microscopic observations or by observation of specific dyes that react with the infected cell. Interpretation is typically based on 50% neutralization, which is the last dilution of serum capable of inhibiting 50% of the total plaques (virions). Plaque reduction neutralization test (PRNT) thresholds of 80 and 90 represent dilutions of sera capable of reducing plaques by 80% or 90% respectively. The results are shown in Table 5.

TABLE 5
Monovalent SARS-CoV-2 VLP Vaccine Neutralizing Antibodies

| Test Group | PRNT50 | PRNT80 | PRNT90 |
|---|---|---|---|
| POV (before vaccination) | ** | | |
| P1VD14 Group 1 | ** | | |
| P2VD14 Group 1 | 2560 | 1280 | 640 |
| P1VD14 Group 2 | 320 | 160 | 80 |
| P2VD14 Group 2 | *** | 5120 | 2560 |
| P1VD14 Group 3 | 320 | 285 | 160 |
| P2VD14 Group 3 | 5120 | 2560 | 1280 |
| P1VD14 Group 4 | 640 | 320 | 160 |
| P2VD14 Group 4 | *** | 5120 | 2560 |
| P1VD14 Group 5 | 2560 | 640 | 320 |
| P2VD14 Group 5 | 5120 | 2276 | 1280 |
| P1VD14 Group 6 | ** | | |
| P2VD14 Group 6 | 5120 | 2560 | 640 |
| P1VD14 Group 7 | 160 | 80 | ** |
| P2VD14 Group 7 | 2560 | 1280 | 320 |

** below lowest dilution limit of PRNT (titer 40);
*** above highest dilution range of PRNT (titer 5120)

As shown in Table 5, all of the monovalent vaccine constructs induced a neutralizing antibody response. This response was very potent, as demonstrated by the data from the stringent PRNT 90 threshold.

### Example 6: Evaluation of the Potency of a Trivalent SARS-CoV-2 VLP Vaccine Construct

A trivalent VLP was prepared using the method in Example 2 with antigen plasmids including all of the following sequences:

i)
(SEQ ID NO: 6)
SARS-CoV-2;
ii)
(SEQ ID NO: 9)
SARS-CoV;
and
iii)
(SEQ ID NO: 12)
MERS.

Vaccine formulations comprising the trivalent VLP, a monovalent VLP (expressing native SARS-CoV-2 (SEQ ID NO. 6), a recombinant SARS-CoV-2 (SEQ ID NO: 25) and Gag protein alone (SEQ ID NO:1) were tested in vivo in mice. The recombinant SARS-CoV-2 (SEQ ID NO: 25) was provided by the National Research Council of Canada. The vaccines were formulated with aluminum phosphate adjuvant (Adjuphos®) as shown in Table 6.

Forty naïve 6-8 week-old C57/BL6 mice (4 groups of 10) were immunized three times with approximately 1/20^{th} to 1/50^{th} of a human dose of the vaccine formulations shown below in Table 6. Immunization took place at day 0, day 21 and day 42. Animals were sacrificed 14 days after the last immunization and their serum was collected for subsequent analysis of anti-spike protein antibody titers and neutralizing antibodies.

TABLE 6
Vaccine Formulations

| | | | | | Dose |
|---|---|---|---|---|---|
| | | μg SARS- | | | volume/ |
| Test | | CoV-2 | μg | Al+++ | Animal |
| Group | Description | Spike/dose | Gag/dose | pg/dose | (μL) |
| 1 | Monovalent SARS-CoV-2 native spike | 0.1 | 50 | 125 | 250 |
| | protein VLP | | | | |
| 2 | Trivalent SARS-CoV-2 (native spike | 0.1 | 50 | 125 | 250 |
| | protein); SARS-CoV; MERS-CoV VLP | | | | |
| 3 | SARS-CoV-2 spike recombinant | 0.1 | N/A | 125 | 250 |
| 4 | Empty Gag | N/A | 50 | 125 | 250 |

Anti-Spike SARS-CoV-2, anti-SARS and anti-MERS antibody titers were measured for each group using the technique described in Example 5 with the following capture antigens (SARS-COV-2 Spike Protein (51+S2), Sino Biological, Cat #40589-V08B1, SARS-COVSpike Protein (51+S2), MyBioSource, Cat #MBS434077 and MERS-CoV Spike Protein (51+S2), Sino Biological, Cat #40069-V08B). The results are shown in Table 7.

TABLE 7
Coronavirus Antibody Titres

| | | Anti SARS- | Anti SARS- | Anti MERS |
|---|---|---|---|---|
| | Time | CoV2 Spike | CoV Spike | Spike |
| Group | Point | binding titre | binding titre | binding titre |
| Pooled Group 1 | P1Vd14 | 2700 | 300 | negative |
| Monovalent | P2Vd14 | 72900 | 8100 | 100 |
| VLP | P3Vd14 | 218700 | 24300 | negative |
| Pooled Group 2 | P1Vd14 | 2700 | 900 | >2700 |
| Trivalent | P2Vd14 | 24300 | 24300 | >72900 |
| VLP | P3Vd14 | >72900 (72900~ | 72900 | 656100 |
| | | 218700) | | |
| Pooled Group 3 | P1Vd14 | 2700 | 900 | negative |
| Stabilized | P2Vd14 | 72900 | 24300 | 900 |
| Recombinant | P3Vd14 | 218700 | 24300 | negative |
| SARS-CoV2 | | | | |
| Pooled Group 4 | P1Vd14 | negative | negative | negative |
| Empty Gag | P2Vd14 | negative | negative | negative |
| VLP | P3Vd14 | negative | negative | negative |

As shown in Table 7, the trivalent VLP (Group 2) induced antibody responses against all three coronaviruses: SARS-CoV-2, SARS and MERS. This demonstrates that a trivalent vaccine candidate has the potential to provide immunological protection again all three major coronaviruses.

Anti-SARS-CoV-2 binding and PRNT 80 neutralizing titres for individual animals after the third vaccination are shown in Table 8 below. Neutralizing antibodies were measured using the method described in Example 5.

TABLE 8
Anti-SARS-CoV-2 Binding and Neutralizing Titres for Individual Mice

| | | | | Anti | | | |
|---|---|---|---|---|---|---|---|
| | | | | SARS-CoV2 | Anti | | |
| | | | Anti | binding | SARS-CoV2 | Neutralizing | nAb/ |
| | | | SARS-CoV2 | titre | Neutralizing | Antibodies | Binding |
| Group | | Mouse | binding | Geometric | Antibodies | Geometric | Titres |
| # | Description | # | titre | Mean | (PRNT 80) | Mean | Ratio |
| 1 | SARS-CoV-2 | 6 | 1,057,003 | 355538 | 2560 | 1810 | 0.002 |
| | native Spike | 7 | 221,691 | | 640 | | 0.003 |
| | protein eVLP | 8 | 515,381 | | 1280 | | 0.002 |
| | | 9 | 240,199 | | 5120 | | 0.021 |
| | | 10 | 124,759 | | 1280 | | 0.01 |
| | | 11 | 1,320,203 | | 5120 | | 0.004 |
| | | 12 | 258,417 | | 1280 | | 0.005 |
| | | 13 | 252,174 | | 640 | | 0.003 |
| | | 14 | 454,574 | | 5120 | | 0.011 |
| | | 15 | 228,032 | | 1280 | | 0.006 |
| 2 | SARS-CoV-2 + | 21 | 333,263 | 247080 | >5120 | >5120 | 0.015 |
| | SARS-CoV- + | 22 | 299,575 | | >5120 | | 0.017 |
| | MERS-CoV | 23 | 208,191 | | >5120 | | 0.024 |
| | Native Spike | 24 | 142,458 | | >5120 | | 0.036 |
| | protein eVLP | 25 | 294,434 | | >5120 | | 0.017 |
| | | 26 | 129,136 | | >5120 | | 0.04 |
| | | 27 | 322,558 | | >5120 | | 0.016 |
| | | 28 | 390,847 | | >5120 | | 0.013 |
| | | 29 | 278,663 | | >5120 | | 0.019 |
| | | 30 | 214,394 | | >5120 | | 0.024 |

As can be seen from the data shown in Table 8 demonstrates that the trivalent VLP induced higher neutralizing antibody responses than the monovalent SARS-CoV-2 VLP even though the binding titres were lower. This is particularly evident when by observing the ratio f neutralizing antibodies to binding antibody titres in the last column of Table 8. This demonstrates that the trivalent vaccine candidate has the potential to provide stronger immunological protection against COVID-19.

The serum obtained from mice fourteen (14) days after each vaccination was tested for cross reactivity with a different coronavirus which is known to infect humans and cause a common cold (HCoV-0C43). Antibody titres were measured using ELISA as described above using human coronavirus (HCoV-0C43) spike protein (S1+S2 ECD, His Tag), Sino, Cat #40607-V08B, stock 0.25 mg/mL as the capture antigen. The results are shown below in Table 9 below.

TABLE 9
Cross Reactivity of Mouse Serum
against HCoV-OC43 Spike Protein

| | | Anti-HCoV-OC43 |
|---|---|---|
| | Vaccination | Spike Total IgG |
| Group | Number | Binding Titres |
| Pooled Group 1 | P1VD14 | negative |
| Monovalent VLPs | P2VD14 | 100 |
| | P3VD14 | 300 |
| Pooled Group 2 | P1VD14 | negative |
| Trivalent VLPs | P2Vd14 | 900 |
| | P3VD14 | 2700 |
| Pooled Group 3 NRC | P1VD14 | negative |
| Stabilized Recombinant | P2VD14 | 300 |
| SARS-CoV2 | P3Vd14 | 300 |
| Pooled Group 4 Empty | P1VD14 | negative |
| eVLP | P2VD14 | negative |
| | P3VD14 | negative |

As can be seen from Table 9 above, the trivalent VLP vaccine candidate (Group 2) demonstrated higher cross reactivity against a human coronavirus which causes common cold. As such, the trivalent candidate demonstrated the potential for broader protection against coronavirus than the monovalent VLP or the recombinant SARS-CoV-2 spike protein alone.

In order to evaluate the efficacy of the vaccine formulations, the neutralizing antibodies were also measured in human serum (HS) collected from four recovered COVID-19 patients and the results were compared to the neutralizing antibodies induced by the four different test groups shown in Table 6. PRNT 50 and PRNT 90 was determined following the first and second vaccination using the method described in Example 5. Pooled results for each group are shown in Table 10 below.

TABLE 10
Anti-SARS-CoV-2 Neutralizing Antibodies

| Sample | PRNT50 | PRNT90 |
|---|---|---|
| HS1 | 80 | ** |
| HS2 | 160 | 40 |
| HS3 | 1280 | 320 |
| HS4 | 160 | 80 |
| POV (BEFORE VACCINATION) | ** | ** |
| P1VD14 GR1 | ** | ** |
| P1VD14 GR2 | ** | ** |
| P1VD14 GR3 | 40 | ** |
| P1VD14 GR4 | ** | ** |
| P2VD14 GR1 | 640 | 160 |
| P2VD14 GR2 | 320 | ** |
| P2VD14 GR3 | 640 | 80 |
| P2VD14 GR4 | ** | ** |

** below lowest dilution limit of PRNT (titer 40);

As can be seen in Table 10, the monovalent VLP vaccine induced more neutralizing antibodies than COVID-19 infection in three out of four human patients as measured by PRNT 50 and 90. The trivalent VLP vaccine induced more neutralizing antibodies than COVID-19 infection in three out of four human patients as measured by PRNT 50. Accordingly, the vaccine constructs at least as effective, and potentially more effective, at inducing immune protection than exposure to SARS-CoV-2.

### Example 7: Evaluation of Protective Effect of a Monovalent SARS-CoV-2 VLP Vaccine Construct

Syrian golden hamsters (males, aged approximately 5-6 weeks old) were divided into two groups and immunized with two doses of the formulations shown below in Table 11, specifically a test sample comprising a triple modified SARS-CoV2 VLP (SEQ ID: 24) formulated with aluminum phosphate adjuvant (Adjuphos®) (Group B) and a saline control (Group A). Immunizations took place at day 0 and day 21, via intramuscular injection. At day 42, all animals were challenged intranasally with 50 μl of SARS-CoV-2 via both nares, at a challenge virus dose of 1×10⁵ TCID₅₀ per animal. SST (serum separation tube) blood samples (approximately 0.5 ml each) were collected on day 0 prior to the prime immunization, day 14 and day 35, respectively. Final blood samples were collected at necropsy. Nasal washes were collected on days 35, 43, 44, 45, 47, 49, 51, 53 and 56. Half of the animals in each group were euthanized at three days post-challenge, and the remaining animals were euthanized at 14 days post-challenge.

TABLE 11
Monovalent SARS-CoV-2 VLP Vaccine Formulations

| | | | | Dose |
|---|---|---|---|---|
| | | μg SARS- | | volume/ |
| | | CoV-2 | Al+++ | Animal |
| Group | Test Article | Spike/dose | μg/dose | (μL) |
| A | Saline control | N/A | N/A | 100 |
| B | SARS-CoV-2 Proline | 1 | 125 | 100 |
| | and Furin Cleavage | | | |
| | Modified with TM/Cyt | | | |
| | from VSV (SEQ ID | | | |
| | NO: 24) | | | |

At necropsy, gross lung pathology was evaluated and the proportion of lung lobe that contained lesions was estimated. Lung tissues were analyzed for viral load by qRT-PCR and viral cell culture. Similarly, nasal turbinates were collected for viral load by qRT-PCR and viral cell culture.

Extraction of RNA from nasal washes was performed using Qiagen reagents (QIAamp Viral RNA Mini Kit Cat No./ID: 52906). Briefly, 140 μl of nasal wash was added into 560 μl viral lysis buffer (Buffer AVL). The mixture was incubated at room temperature for 10 min. After brief centrifugation, the solution was transferred to a fresh tube containing 560 μL of 100% ethanol, and the tube was incubated at room temperature for 10 min. RNA was then purified and eluted with 60 μl of RNase Free water containing 0.04% sodium azide (elution buffer AVE).

Extraction of RNA from lung lobes and nasal turbinates was completed using approximately 100 μg of tissue. The tissues were homogenized in 600 μl of lysis buffer (RLT Qiagen) with a sterile stainless steel bead in the TissueLyserII (Qiagen) for 6 min, at 30 Hz. The solution was centrifuged at 5000×g for 5 min. Supernatant was transferred to a fresh tube containing 600 μl of 70% ethanol, and the tube was incubated at room temperature for 10 min. Viral RNA was then purified using Qiagen RNeasy Mini Kit (Cat No/ID: 74106) and eluted with 50 μL elution buffer.

qRT-PCR assays were performed on RNA from samples of nasal washes, lung tissues and nasal turbinates using SARS-CoV-2 specific primers (Table 12). The primers had an annealing temperature of approximately 60° C. Qiagen Quantifast RT-PCR Probe kits were used for qRT-PCR, and the qRT-PCR reactions were conducted using the OneStep Plus (Applied Biosystems) machine. The qRT-PCR results were expressed in copy number per reaction, by producing a standard curve with a sample of a linearized plasmid DNA that contains the env gene of SARS-CoV-2. The Ct values for individual samples were used with the standard curve to determine the copy number in each sample.

TABLE 12
Sequence of Primers Used

| Primer | Sequence | SEQ ID NO: |
|---|---|---|
| Forward Primer (Fwd) | ACAGGTACGTTAATAGTTAATAGCGT | 31 |
| Reverse Primer (Rev) | ATATTGCAGCAGTACGCACACA | 32 |
| Labelled Probe | ACACTAGCCATCCTTACTGCGCTTCG | 33 |

Viral titration assays were performed to assess infectious virus. The assays were conducted in 96-well plates using Vero′76 cells (ATCC CRL-1587). Median tissue culture infectious dose (TCID₅₀) was determined by microscopic observation of the cytopathic effect (CPE) of cells. The virus was quantified and reported in TCID₅₀/ml or TCID₅₀/gram. TCID₅₀ values were calculated using the Spearman & Karber algorithm in Excel.

Anti-Spike SARS-CoV-2 antibody titers were measured by ELISA performed on serum samples. Plates were coated with spike S1+S2 Ag (Cat #40589-V08B1, Sino Biological Inc.). The coating concentration was 0.1 ug/mL. Plates were blocked with 5% non-fat skim milk powder in PBS containing 0.05% Tween 20. Fourfold dilutions of serum were used. Goat anti-Hamster IgG HRP from ThermoFisher (PA1-29626) was used as the secondary antibody at 1:7000. Plates were developed with OPD peroxidase substrate (0.5 mg/ml) (Thermo Scientific Pierce 34006). The reaction was stopped with 2.5 M sulfuric acid and absorbance was measured at 490 nm. Throughout the assay, plates were washed with PBS containing 0.05% Tween 20. The assay was performed in duplicate. The titres were reported as the end point of the dilutions.

Antibodies to the spike protein receptor binding domain (“RBD”) were measured as follows. Anti-SARS-CoV-2 spike 51 RBD IgG antibody binding titer was determined from serum samples using an indirect ELISA. Recombinant SARS-CoV-2 spike 51 RBD protein was adsorbed on a microtiter plate overnight and plates were then blocked with a solution of 5% skim milk in wash buffer for 1 hour. After blocking and washing, samples were added to the microplates and incubated for 1.5 hours. An HRP-conjugated goat anti-Syrian Hamster IgG-Fc was used as a detection antibody, and incubated on the microplates for 1 hour. The signal was developed with Tetramethylbenzidine (TMB) substrate solution and the reaction stopped by addition of 450 μL Liquid Stop Solution for TMB Microwell Substrate. The absorbance was read at 450 nm using an ELISA microwell plate reader.

Viral neutralization assays against the challenge SARS-CoV-2 virus were performed on the serum samples using the cell line Vero′76. The serum samples were heat-inactivated for 30 min at 56° C. The serum samples were serially diluted (2-fold serial dilutions). The experiment was conducted in technical duplicates. The virus was diluted in medium to a concentration of 25 TCID₅₀ in 50 μl per well (the inoculum size=25 TCID₅₀). Then 60 μl of the virus solution was mixed with 60 μl serially diluted serum samples. The mixture was incubated for 1 hr at 37° C., with 5% CO₂. The pre-incubated virus-serum mixtures (100 μl) were transferred to the wells of the 96-well flat-bottom plates containing 90% confluent pre-seeded Vero′76 cells. The plates were incubated at 37° C., with 5% CO₂ for five days. The plates were observed using a microscope on day 1 post-infection (dpi) for contamination and on days 3 and 5 post-infection for cytopathic effect. The serum dilution factor for the wells with no CPE at 5 dpi was defined as the serum neutralization titre. The initial serum dilution factor was 1:20.

Neutralizing antibodies were tested as follows. Vero cells were seeded at 8×10⁵ cells/well in 6-well plates 48 h prior to infection. Sera were heat-inactivated at 56° C. for 30 min then transferred on ice. Sera were diluted 1:10 with virus infection media then each diluted serum was used to carry out ½× fold serial dilutions to give 1:20 to 1:40960 (8 subsequent dilutions). Equal volumes of diluted serum and virus (100 pfu per serum dilution) were mixed and incubated at 37° C. for 1 h. No sera and no virus controls were included. Cells were washed with PBS and each virus/serum were transferred and mixed to each well containing cells, and incubated at 37° C. for 1 h, with interval rocking of the plates. After the 1 h adsorption, excess inoculum was removed and a 2 ml virus infection media/agarose mix were overlaid onto the cells. The overlay was allowed to solidify and plates were incubated at 37° C. for 72 h. Cells were stained with crystal violet at 72 h post-infection. Plaques were quantified for all dilutions and PRNT titer was calculated. The % plaque reduction for all the dilutions based on the no serum control, was calculate using the Reed-Muench formula to determine the PRNT titers 50, 80, and 90.

Lung tissues were also quantified for cytokine gene expression collected at necropsy. The gene expression of IL-4, IL-10, IL-13, TNF-alpha and IFN-gamma was determined in the right cranial and right caudal lung lobe by qRT-PCR using the primers shown in Table 13. The beta-actin gene expression was used for reference.

TABLE 13
Primer Sequence

| Gene | | SEQ | | SEQ |
|---|---|---|---|---|
| Target | Forward Primer 5′→3′ | ID NO: | Reverse Primer 5′→3′ | ID NO: |
| IL-4 | CCACGGAGAAAGACCTCA | 34 | GGGTCACCTCATGTTGGAAA | 40 |
| | TCTG | | TAAA | |
| IL-10 | GTTGCCAAACCTTATCAGA | 35 | TTCTGGCCCGTGGTTCTCT | 41 |
| | AATGA | | | |
| IL-13 | AAATGGCGGGTTCTGTGC | 36 | AATATCCTCTGGGTCTTGTAG | 42 |
| | | | ATGG | |
| TNF- | GGAGTGGCTGAGCCATCGT | 37 | AGCTGGTTGTCTTTGAGAGA43 | |
| alpha | | | CATG | |
| IFN- | GGCCATCCAGAGGAGCAT | 38 | TTTCTCCATGCTGCTGTTGAA | 44 |
| gamma | AG | | | |
| Beta- | | | | |
| actin | ACTGCCGCATCCTCTTCCT | 39 | TCGTTGCCAATGGTGATGAC | 45 |

Lung tissues were collected in RNAlater and the RNA was isolated using Qiagen RNeasy Mini extraction kits using RLT lysis buffer (Qiagen RNeasy Mini Kit, Cat No/ID:74106). RNA concentration and the 260/280 ratio as an indicator of purity was determined by a nanodrop spectrophotometer. cDNA was synthesized using iScript™ Reverse Transcription Supermix with 500 ng of RNA as template. cDNA was synthesized following a program of 5 min at 25° C., 20 min at 46° C., and 95° C. for 1 min. Master mix was prepared for each gene of interest as well as a house keeping gene at 10% overage: 1.84 μl Nuclease Free H₂O; Forward Primer 0.08 μL; Reverse Primer 0.08 μl; and SYBR 10 μl [SYBR® Green PCR Master Mix (SsoAdvanced™ Universal SYBR® Green Supermix #1725275)]. Twelve μl of the master mix was combined with 8 μl of RNA for each PCR reaction. After loading, the plate was centrifuged at 1500 RPM for 1 min to bring all liquid back into base of well. The qPCR was performed using a Bio-Rad Thermocycler (Bio-Rad CX1000). Data was analyzed using the Bio-Rad CFX Maestro software. Data is exported in the form of Ct values to an excel spreadsheet for fold change calculation by ΔΔCt Formula in Excel.

Results based on clinical observation of animals indicated that all animals were healthy throughout the immunization phase. All animals had normal activity levels and had no clinical signs. The body weight increases were normal in the group vaccinated with test article (Group B) when compared to the Saline control group (Group A).

Immune response to vaccination as measured by antibody titres to SARS-CoV-2 spike protein are shown in Table 14 fourteen days after the first vaccination and fourteen days after the second vaccination (P1 and P2 refer to the first and second vaccination). Results shown are Geo means of the animals in each group.

TABLE 14
SARS-CoV-2 VLP Vaccine Antibody Titres

| | First | | Second | |
|---|---|---|---|---|
| | Vaccination (day 14) | | Vaccination (day 14) | |
| | Anti- | Anti- | Anti- | Anti- |
| | SARS- | SARS- | SARS- | SARS- |
| | CoV-2 | CoV-2 | CoV-2 | CoV-2 |
| | S1 | Spike | S1 | Spike |
| | RBD | total IgG | RBD | Total IgG |
| Group A | — | 3 | — | 3 |
| (control) | | | | |
| Group B | 7,446 | 1,222 | 268,399 | 22,868 |
| (SARS-CoV-2 | | | | |
| Proline and | | | | |
| Furin Cleavage | | | | |
| Modified with | | | | |
| TM/Cyt from VSV | | | | |
| (SEQ ID NO: 24) | | | | |

The Group B animals (immunized with the triple modified monovalent SARS-CoV-2 VLP vaccine) produced high levels of anti-spike antibody two weeks after the second vaccination. At two weeks after the first vaccination, 10 out of 12 animals in Group B produced anti-spike antibodies (data not shown). Group A animals (Saline control) did not have anti-spike antibody production. The triple modified monovalent SARS-CoV-2 VLP vaccine also induced detectable level of anti-SARS-CoV-2-S1 RBD IgG antibody at 14 days after the first immunization. A substantial increase in antibody titres was observed on day 14 after the 2^{nd} immunization. No anti-SARS-CoV-2-S1 RBD IgG were detected in control Group A.

The neutralizing antibodies, as determined by PRNT, for each group fourteen days after the first vaccination are shown in Table 15 (average values shown). Values indicate reciprocal of highest dilution that showed inhibition of 50% (PRNT50), 80% (PRNT80), or 90% (PRNT90) of input virus, respectively.

TABLE 15
SARS-CoV-2 VLP Vaccine Neutralizing Antibodies

| Test Group - Fourteen days | | | |
|---|---|---|---|
| after first vaccination | PRNT50 | PRNT80 | PRNT90 |
| Group A Pooled | ** | (N/A) | (N/A) |
| (Control) | | | |
| Group B | 441 | 241 | 198 |
| (SARS-CoV-2 Proline and | | | |
| Furin Cleavage Modified | | | |
| with TM/Cyt from VSV | | | |
| (SEQ ID NO: 24) | | | |

** below lowest dilution limit of PRNT (titer 40)

All Group B animals produced virus neutralizing antibodies at two weeks post-immunization as shown in Table 15. The Group A animals did not produce any neutralizing antibodies as shown in Table 15.

At three days post-challenge (dpc), all animals Group B, and only one animal in Group A, produced neutralizing antibodies (data not shown). At 14 dpc, all the animals in Group A and B produced neutralizing antibodies. (data not shown).

During the challenge phase, all animals except for two were active and had normal activity levels, and did not have abnormal nasal signs.

Animals were weighed each day post challenge. After challenge, Group A animals lost approximately 15% of their initial body weight, peaking at 6-8 dpc. The means of % body weight changes of the Group B animals were only about 1-2% and peaked at two dpc. Body weight data is shown in Table 16 below at Day 0 and at Day 3 and 6 after challenge.

TABLE 16
Body Weight of Hamsters following Viral Challenge

| | | Day 0 | | Day 3 | | Day 6 | |
|---|---|---|---|---|---|---|---|
| | Animal | | BW | | BW | | BW |
| Group | ID | BW | Average | BW | Average | BW | Average |
| A | 401 | 175.1 | 171.5 ± 14.5 | 160.9 | 160.9 ± 13.9 | Euth | 155.5 ± 11.5 |
| (control) | 403 | 182.1 | | 167.3 | | 155.4 | |
| | 408 | 172.6 | | 160.7 | | Euth | |
| | 412 | 173.6 | | 161 | | 149.1 | |
| | 416* | 165.9 | | 163.2 | | 137.1 | |
| | 422 | 196.3 | | 185.8 | | 170.5 | |
| | 423 | 162.8 | | 149.6 | | Euth | |
| | 429 | 185.2 | | 171.6 | | 159.6 | |
| | 434 | 174.8 | | 170.00 | | 161.5 | |
| | 435 | 156.4 | | 143.7 | | Euth | |
| | 440 | 174.3 | | 164.9 | | Euth | |
| | 443 | 139.4 | | 132.1 | | Euth | |
| B | 402 | 159.5 | 174.2 ± 14.3 | 155.7 | 171.6 ± 14.3 | Euth | 180.3 ± 7.9 |
| (SARS-CoV-2 | 404 | 181.2 | | 177.1 | | 179.6 | |
| Proline and | 410 | 161.4 | | 160.1 | | Euth | |
| Furin Cleavage | 411 | 190.0 | | 183.6 | | 182.1 | |
| Modified with | 418 | 181.3 | | 179.1 | | 185.1 | |
| TM/Cyt from | 421* | 188.4 | | 189.5 | | 191.6 | |
| VSV (SEQ ID | 426 | 160.0 | | 159.0 | | Euth | |
| NO: 24) | 428 | 175.8 | | 173.3 | | 172.8 | |
| | 431 | 172.3 | | 169.1 | | 170.3 | |
| | 436 | 152.7 | | 147.3 | | Euth | |
| | 442 | 199.1 | | 195.4 | | Euth | |
| | 446 | 168.8 | | 169.9 | | Euth | |

Euthanized on day 3 as planned or humane euthanized
*(416 and 421)

As can be seen in Table 16, animals given the Saline solution lost considerable weight three days and six days after challenge whereas the animals that had received the vaccine lost considerably less weight at day 3 and were had gained weight by day 6.

Viral RNA as measured in nasal washes post challenge is shown in Table 17. In all days examined, the vaccinated (Group B) animals had lower viral RNA levels in nasal washes than the Group A animals (control group), as depicted in Table 17 (showing copies/Rxn for each day post-challenge). Only during day two after challenge were the viral RNA levels significantly lower in Groups B compared to Group A (p=0.0206).

TABLE 17
Viral RNA in nasal washes

| | Table 16 Day post-challenge Viral RNA Averages (copies/Rxn) | | | | | | | |
|---|---|---|---|---|---|---|---|---|
| Group ID | 0 | 2 | 3 | 5 | 7 | 9 | 11 | 14 |
| Group A | 0.1 | 3708602 | 340897 | 94932 | 12409 | 6231 | 130 | 3146 |
| Group B | 0.1 | 205616 | 105469 | 31861 | 93 | 20 | 2461 | 2 |

Viral RNA at 3 days post-challenge in various tissues for control (Group A) and vaccinated (Group B) animals are shown in Table 18 (showing values for copies/gram). At three days post-challenge, viral RNA was detectable in the right cranial lobe (RCra) and the right caudal lobe (RCau) of the lung and the nasal turbinates in all animals. When compared to Group A, the levels of viral RNA in the RCra of Group B were significantly lower. Similarly, the levels of RNA in RCau were significantly lower in Group B than Group A. In the nasal turbinates, viral RNA levels in Group B was significantly lower than in Group A.

TABLE 18
Viral RNA in tissues at 3 days post-challenge

| | Right cranial | Right caudal | Nasal |
|---|---|---|---|
| Group ID | lobe | lobe | turbinates |
| Group A | 2613650533 | 1608083300 | 2099767910 |
| (control) | | | |
| Group B | 108006 | 42465 | 1205108163 |
| (SARS-CoV-2 Proline and | | | |
| Furin Cleavage Modified | | | |
| with TM/Cyt from VSV | | | |
| (SEQ ID NO: 24) | | | |

Viral RNA at 14 days post-challenge in various tissues for the control (Group A) and vaccinated (Group B) animals are shown in Table 19 (showing values for copies/gram). At 14 days post-challenge, viral RNA was detectable in all Group A animals and some animals in Group B in the RCra, RCau or nasal turbinates. The levels of RNA in RCra and RCau were significantly different in Group B than those in Group A.

TABLE 19
Viral RNA in Tissues at 14 days Post-challenge

| | Right cranial | Right caudal | Nasal |
|---|---|---|---|
| Group ID | lobe | lobe | turbinates |
| Group A | 127855.5 | 62075.0 | 3104415.2 |
| (control) | | | |
| Group B | 99.43 | 105.78 | 100398.57 |
| (SARS-CoV-2 Proline and | | | |
| Furin Cleavage Modified | | | |
| with TM/Cyt from VSV | | | |
| (SEQ ID NO: 24) | | | |

Infectious virus in various tissues at 3 days post-challenge for control (Group A) and vaccinated (Group B) animals are shown in Table 20 (showing values for TCID₅₀/gram). At three days post-challenge, infectious virus was detectable in all animals of Group A in the right cranial and right caudal lobes of the lung and in nasal turbinates. The titres of infectious virus in Group B was significantly lower than those in Group A. At 14 days post-challenge, infectious virus was not detected in any of the animals (data not shown).

TABLE 20
Infectious Virus in tissues at 3 days post-challenge (TCID50/gram)

| | Right cranial lobe | | Right caudal lobe | | Nasal turbinates | |
|---|---|---|---|---|---|---|
| Group ID | TCID50/gram | Average | TCID50/gram | Average | TCID50/gram | Average |
| Group A | 1.51E+07 | 2.63E+07 | 6.71E+02 | 1.65E+06 | 1.15E+07 | 2.00E+07 |
| (control) | 1.03E+07 | | 3.94E+05 | | 2.39E+06 | |
| | 3.89E+07 | | 3.45E+05 | | 3.76E+07 | |
| | 1.30E+07 | | 1.23E+06 | | 4.66E+05 | |
| | 1.32E+06 | | 5.99E+06 | | 2.47E+07 | |
| Group B | 1.79E+01 | 1.48E+01 | 8.78E+00 | 3.45E+00 | 1.26E+02 | 3.58E+06 |
| (SARS-CoV-2 | 1.49E+01 | | 2.56E+00 | | 5.43E+05 | |
| Proline and | 2.39E+01 | | 2.28E+00 | | 5.86E+01 | |
| Furin Cleavage | 9.05E+00 | | 2.42E+00 | | 2.09E+07 | |
| Modified with | 1.12E+01 | | 2.59E+00 | | 6.85E+00 | |
| TM/Cyt from | | | | | | |
| VSV (SEQ ID | | | | | | |
| NO: 24) | | | | | | |

Heavier lungs is associated with more advanced disease. Therefore, the ratio of lung weight to body is correlated with more severe disease states. Table 21 shows the lung weight to body weight ratios for animals in the control (Group A) and vaccinated (Group B) animals three days post challenge. Animals in group Group B animals had significantly lower lung weight to body weight ratios.

TABLE 21
Ratios of lung weight to body weight (%)

| | Lung | Body | Lung weight/ | |
|---|---|---|---|---|
| | Weight | Weight | Body Weight | Average ± |
| Group ID | (g) | (g) | Ratio | SD |
| Group A | 1.4 | 160.9 | 0.87 | 0.76 ± 0.15 |
| (control) | 1.2 | 170.1 | 0.71 | |
| | 1.5 | 160.7 | 0.93 | |
| | 1.1 | 160.9 | 0.68 | |
| | 0.88 | 137.1 | 0.64 | |
| | 1.4 | 183.7 | 0.76 | |
| | 1.6 | 149.6 | 1.07 | |
| | 1.3 | 183.6 | 0.71 | |
| | 1 | 182.3 | 0.55 | |
| | 1.1 | 143.7 | 0.77 | |
| | 1.3 | 164.9 | 0.79 | |
| | 0.8 | 132.1 | 0.61 | |
| Group B | 0.9 | 155.7 | 0.58 | 0.56 ± 0.07 |
| (SARS-CoV-2 | 1 | 188.2 | 0.53 | |
| Proline and Furin | 1 | 160.1 | 0.62 | |
| Cleavage Modified | 0.9 | 188.2 | 0.48 | |
| with TM/Cyt from | 0.9 | 195.2 | 0.46 | |
| VSV (SEQ ID | 0.98 | 191.6 | 0.51 | |
| NO: 24) | 1 | 159 | 0.63 | |
| | 0.9 | 180.1 | 0.50 | |
| | 1 | 183.6 | 0.54 | |
| | 1 | 147.3 | 0.68 | |
| | 1.2 | 195.4 | 0.61 | |
| | NA | 169.9 | missing | |

Following necropsy, lung tissues were fixed in formalin, embedded, sectioned and stained with hematoxylin and eosin (H&E). Slides were examined by a board-certified pathologist and scored on a scale of 0-4 as shown in Table 22.

TABLE 22
Lung Histology Scores (median)

| | | | Intensity of | Extent of | | |
|---|---|---|---|---|---|---|
| | | Proportion of | the inflammatory | hypertrophy | | |
| Group | Days Post- | parenchyma | infiltrate in | of alveolar | Interalveolar | Extent of |
| ID | Challenge | affected | affected areas | pneumocytes | hemorrhage | emphysema |
| Group A | 3 | 3 | 3 | 0 | 2 | 0 |
| (control) | | 3 | 3 | 0 | 1 | 0 |
| | | 3 | 3 | 0 | 2 | 0 |
| | | 1 | 3 | 0 | 3 | 0 |
| | | 1 | 3 | 0 | 2 | 0 |
| | | 1 | 2 | 0 | 1 | 0 |
| | 14 | 1 | 2 | 3 | 0 | 0 |
| | | 1 | 2 | 2 | 0 | 0 |
| | | 4 | 4 | 3 | 4 | 0 |
| | | 4 | 2 | 2 | 0 | 0 |
| | | 2 | 3 | 2 | 0 | 0 |
| | | 1 | 2 | 1 | 0 | 0 |
| Group B | 3 | 0 | 0 | 0 | 0 | 1 |
| (SARS-CoV-2 | | 1 | 2 | 0 | 0 | 0 |
| Proline and | | 1 | 1 | 0 | 0 | 0 |
| Furin Cleavage | | 1 | 2 | 0 | 0 | 0 |
| Modified with | | 1 | 1 | 0 | 1 | 0 |
| TM/Cyt from | | 1 | 1 | 0 | 0 | 0 |
| VSV (SEQ ID | 14 | 0 | 0 | 0 | 0 | 0 |
| NO: 24) | | 0 | 0 | 0 | 0 | 0 |
| | | 0 | 0 | 0 | 0 | 0 |
| | | 1 | 1 | 0 | 0 | 0 |
| | | 0 | 0 | 0 | 0 | 0 |
| | | 0 | 0 | 0 | 0 | 0 |

As can be seen in Table 22, animals in the control group (Group A) showed significant disease pathology following challenge at days 3 and 14. By way of contrast, the vaccinated animals (Group B) showed some minor pathology at day 3 but were mostly recovered by day 14. Accordingly, the vaccine provided significant protection against disease induced lung pathology.

Immunohistochemical staining was conducted to observe SARS-CoV-2 virus in the lung tissues, specifically the parenchyma and bronchioles/bronchi. Staining was observed and scores for the two groups of animals is shown in Table 23.

TABLE 23
Immunohistochemical scores (median)
of the lung for SARS-CoV-2

| | | Parenchyma IHC | | Bronchioles/bronchi | |
|---|---|---|---|---|---|
| | | Indi- | | Indi- | |
| Group | Days post- | vidual | | vidual | |
| ID | challenge | Score | Median | Score | Median |
| Group A | 3 | 4 | 4 ± 0.84 | 2 | 3 ± 0.52 |
| (Control) | | 4 | | 3 | |
| | | 4 | | 2 | |
| | | 4 | | 3 | |
| | | 3 | | 3 | |
| | | 2 | | 3 | |
| | 14 | 0 | 0±1.21 | 2 | 2±0.75 |
| | | 0 | | 2 | |
| | | 3 | | 1 | |
| | | 1 | | 1 | |
| | | 0 | | 3 | |
| | | 0 | | 2 | |
| Group B | 3 | 0 | 0 ± 0 | 2 | 1 ± 0.89 |
| (SARS-CoV-2 | | 0 | | 1 | |
| Proline and | | 0 | | 2 | |
| Furin Cleavage | | 0 | | 0 | |
| Modified with | | 0 | | 1 | |
| TM/Cyt from | | 0 | | 0 | |
| VSV (SEQ ID | 14 | 0 | 0 ± 0 | 0 | 1 ± 0.63 |
| NO: 24) | | 0 | | 1 | |
| | | 0 | | 2 | |
| | | 0 | | 1 | |
| | | 0 | | 1 | |
| | | 0 | | 1 | |

Vaccinated animals had significantly less virus stain in both parenchyma and bronchioles/bronchi than those of the saline control animals (Group A). At 14 days post-challenge, virus stain was similar among the groups in either parenchyma or bronchioles/bronchi although still a little lower in the vaccinated group.

The transcriptional levels of cytokines IL-4, IL-10, IL-13, TNF-alpha and IFN-gamma in the right cranial lung, right caudal lung and the nasal turbinates were determined by qRT-PCR. At 3 days post-challenge, IL-10, IL-13 and IFN-gamma displayed differential expression in the right cranial lobe and the right caudal lobe in Group B (shown in Tables 24 and 25). In nasal turbinates, IL-10 and IFN-gamma exhibited differential expression (shown in Table 26). At 14 days post-challenge, the transcriptional levels of IL-4, IL-10, IL-13, TNF-alpha and IFN-gamma in the right cranial lung, right caudal lung and the nasal turbinates were similar across the groups (shown in Tables 27-29).

TABLE 24
Transcriptional profiles of cytokines in right cranial
lobe 3 days post-challenge (fold changes)

| Group ID | IL-4 | IL-10 | IL-13 | TNF-alpha | IFN-gamma |
|---|---|---|---|---|---|
| Group A | 1.04 | 1.09 | 1.3 | 1.07 | 1.14 |
| Group B | 1.8 | 0.37 | 7.1 | 0.61 | 0.27 |

TABLE 25
Transcriptional profiles of cytokines in right caudal
lobe 3 days post-challenge (fold changes)

| Group ID | IL-4 | IL-10 | IL-13 | TNF-alpha | IFN-gamma |
|---|---|---|---|---|---|
| Group A | 1.02 | 1.03 | 1.33 | 1.04 | 1.21 |
| Group B | 0.88 | 0.33 | 2.4 | 0.55 | 0.35 |

TABLE 26
Transcriptional profiles of cytokines in nasal
turbinates 3 days post-challenge (fold changes)

| Group ID | IL-4 | IL-10 | IL-13 | TNF-alpha | IFN-gamma |
|---|---|---|---|---|---|
| Group A | 1.22 | 1.05 | 1.26 | 1.16 | 1.09 |
| Group B | 1.44 | 0.84 | 2.87 | 0.45 | 0.82 |

TABLE 27
Transcriptional profiles of cytokines in right cranial
lobe 14 days post-challenge (fold changes)

| Group ID | IL-4 | IL-10 | IL-13 | TNF-alpha | IFN-gamma |
|---|---|---|---|---|---|
| Group A | 1.07 | 1.25 | 1.57 | 1.34 | 1.24 |
| Group B | 1.37 | 0.92 | 2.22 | 0.77 | 0.61 |

TABLE 28
Transcriptional profiles of cytokines in right caudal
lobe 14 days post-challenge (fold changes)

| Group ID | IL-4 | IL-10 | IL-13 | TNF-alpha | IFN-gamma |
|---|---|---|---|---|---|
| Group A | 1.09 | 1.51 | 1.13 | 1.11 | 1.31 |
| Group B | 1.38 | 0.78 | 0.63 | 0.94 | 0.65 |

TABLE 29
Transcriptional profiles of cytokines in nasal turbinates
14 days post-challenge (fold changes)

| Group ID | IL-4 | IL-10 | IL-13 | TNF-alpha | IFN-gamma |
|---|---|---|---|---|---|
| Group A | 1.46 | 1.45 | 1.43 | 1.41 | 1.46 |
| Group B | 0.73 | 0.72 | 1.40 | 0.65 | 0.57 |

### Example 8: Evaluation of Potency and Protective Effect of Single Dose of Monovalent SARS-CoV-2 VLP Vaccine Construct

Syrian golden hamsters (males, aged approximately 6-7 weeks old) were immunized with the monovalent triple modified SARS-CoV-2 VLP vaccine formulations shown below in Table 30. Immunizations took place only at day 21 via intramuscular injection. Serum was collected at day 0 and day 35 for subsequent analysis of neutralizing antibodies.

TABLE 30
Monovalent SARS-CoV-2 VLP Vaccine Formulations

| | | μg | | Dose |
|---|---|---|---|---|
| | | SARS- | | volume/ |
| | | CoV-2 | Al+++ | Animal |
| Group | Test Article | Spike/dose | μg/dose | (μL) |
| A | Saline control | N/A | N/A | 100 |
| B | SARS-CoV-2 Proline and | 1.4 | 125 | 100 |
| | Furin Cleavage Modified | | | |
| | with TM/Cyt from VSV | | | |
| | (SEQ ID NO: 24) | | | |

Neutralizing antibodies were tested using the plaque reduction neutralization test (PRNT), as described in Example 7 for animals in Group B. The results are shown in Table 31 (average values shown).

TABLE 31
Monovalent SARS-CoV-2 VLP Vaccine Neutralizing Antibodies

| Test Group | PRNT50 | PRNT80 | PRNT90 |
|---|---|---|---|
| Day 0 (before vaccination) | ** | — | — |
| Day 14 after vaccination | 190 | 65 | 45 |
| Group B | | | |

** below lowest dilution limit of PRNT (titer 40)

Compared to Group B of Example 7 (where animals received immunizations of 1 μg SARS-CoV-2 Spike/dose at day 0 and day 21), animals in Group B of this Example 8 (where animals received a single immunization of 1.4 μg SARS-CoV-2 Spike/dose at day 21) exhibited a higher serum neutralizing antibody response. These data support effective immunization with only a single dose of monovalent SARS-CoV-2 VLP vaccine.

Challenge studies were performed on day 42, as described in Example 7. Table 32 shows average body weights (grams) of animals before challenge.

TABLE 32
Pre-Challenge Average Body Weights of Animals

| Day | Group A | Group B |
|---|---|---|
| −1 | 113.8 | 116.6 |
| 7 | 131.7 | 134.6 |
| 10 | 136.3 | 139.9 |
| 22 | 154.5 | 157.6 |
| 28 | 160.4 | 164.0 |
| 35 | 168.4 | 172.0 |
| 42 | 174 | 178.5 |

Table 33 shows average body weights (grams) of animals post-challenge. As can be seen in Table 33, animals who received a single dose of vaccine lost less weight than those who received saline.

TABLE 33
Post-Challenge Average Body Weights of Animals

| Day | Group A | Group B |
|---|---|---|
| 1 | 172.1 | 174.6 |
| 2 | 168.3 | 172.6 |
| 3 | 165.3 | 173.4 |
| 4 | 163.7 | 168.5 |
| 5 | 159.1 | 168.5 |
| 6 | 155.7 | 168.5 |
| 7 | 154.7 | 170.1 |
| 8 | 157.5 | 171.7 |
| 9 | 162.9 | 173.8 |
| 10 | 164.4 | 174.0 |
| 11 | 166.6 | 175.3 |
| 12 | 169.4 | 177.1 |
| 13 | 172.0 | 177.8 |
| 14 | 173.5 | 178.9 |

Table 34 shows average % change in body weights of animals post-challenge.

TABLE 34
Post-Challenge Average % Body Weight Change of Animals

| Day | Group A | Group B |
|---|---|---|
| 1 | −1.09 | −2.24 |
| 2 | −3.29 | −3.30 |
| 3 | −5.08 | −2.90 |
| 4 | −6.51 | −2.10 |
| 5 | −9.14 | −2.15 |
| 6 | −11.11 | −2.11 |
| 7 | −11.64 | −1.22 |
| 8 | −10.05 | −0.31 |
| 9 | −7.03 | 0.87 |
| 10 | −6.22 | 0.93 |
| 11 | −4.94 | 1.63 |
| 12 | −3.35 | 2.69 |
| 13 | −1.89 | 3.09 |
| 14 | −1.00 | 3.74 |

These data demonstrate that a single immunization of 1.4 μg SARS-CoV-2 Spike/dose at day 21 was effective at preventing reduction in body weight following viral challenge, relative to control.

### Example 9: Evaluation of Monovalent and Trivalent SARS-CoV-2 VLP Vaccine Constructs for Antibody Titers Against South African SARS-CoV-2 Variant

Monovalent and trivalent SARS-CoV-2 VLP vaccine constructs which have the triple modified SARS-CoV-2 spike protein were assessed for production of antibodies against South African SARS-CoV-2 variant. Mice were immunized IP twice (on day 0 and day 21, as described in Example 6) with the SARS-CoV-2 VLP vaccine formulations shown below in Table 35. Animals were sacrificed 14 days after immunization and their serum was collected for subsequent analysis of anti-spike protein antibody titers.

The SARS-CoV-2 VLPs were formulated with aluminum phosphate adjuvant (Adjuphos®) as shown in Table 35.

TABLE 35
SARS-CoV-2 VLP Vaccine Formulations

| | | μg | | Dose |
|---|---|---|---|---|
| | | SARS- | | volume/ |
| | | CoV-2 | Al+++ | Animal |
| Group | Test Article | Spike/dose | μg/dose | (μL) |
| 1 | Monovalent VLP - SARS- | 0.2 | 125 | 500 |
| | CoV-2 Proline and Furin | | | |
| | Cleavage Modified with | | | |
| | TM/Cyt from VSV (SEQ | | | |
| | ID NO: 24) | | | |
| 2 | Trivalent VLP (v4) - | 0.2 | 125 | 500 |
| | SARS-CoV-2 Spike Protein | | | |
| | (described in Example 4) | | | |

Antibody titers were assessed by ELISA, as described in Example 7, except that well plates were coated with SARS-COV-2 Spike Protein from South African variant. Antibody titers at 14 days after the second immunizations are shown in Table 36. Results shown are Geo means of the animals in each group.

TABLE 36
SARS-CoV-2 VLP Vaccine Antibody Titres

| | | | Anti- SARS- |
|---|---|---|---|
| | | Anti- SARS- | CoV-2 African |
| | Days post | CoV-2 Spike | Variant Spike |
| | Second | Total IgG | Total IgG |
| Group | Vaccination | Binding Titers | Binding Titers |
| Group 1 | 14 | 128,850 | 121,511 |
| Group 2 | 14 | 215,232 | 211,080 |

These data demonstrate that mice injected with the monovalent and trivalent vaccines produced antibodies which bind to the South African variant of the Spike protein of SARS-CoV.

### Example 10: Evaluation of Isotype Antibody Titer of Monovalent SARS-CoV-2 VLP Vaccine Construct

In another study, the isotype of antibody titers were assessed, following immunization of mice with the vaccine constructs shown in Table 37.

TABLE 37
SARS-CoV-2 VLP Vaccine Antibody Titres

| | | μg | | Dose |
|---|---|---|---|---|
| | | SARS- | | volume/ |
| | | CoV-2 | Al+++ | Animal |
| Group | | Spike/dose | μg/dose | (μL) |
| 1 | Monovalent Native | 0.2 | 125 | 500 |
| | SARS-Cov-2 (SEQ ID | | | |
| | NO: 6) VLP | | | |
| 2 | Stabilized Recombinant | 0.2 | 125 | 500 |
| | SARS-CoV2 spike | | | |
| | protein (non-VLP) | | | |

Mice were immunized IP twice (on day 0 and day 21, as described in Example 6). Animals were sacrificed 14 days after immunization and their serum was collected for subsequent analysis of anti-spike protein antibody titers.

As shown in Table 38, unexpectedly, when VLPs were formulated with the same amount/concentration of alum as recombinant spike protein, a balanced antibody response was seen (IgG1/IgG2b). Increased production of IgG2b is associated with a TH1 immune response, which is indicative of cell-mediated immunity. This indicates that the VLP construct resulted in elevated levels of IgG2b expression which is correlated to the more effective TH1 immune response.

TABLE 38
SARS-CoV-2 VLP Vaccine Antibody Titres

| | | Anti- SARS- | Anti- SARS- |
|---|---|---|---|
| | Days post | CoV-2 Spike | CoV-2 Spike |
| | second | IgG1 | IgG2b |
| Group | vaccination | Binding Titers | Binding Titers |
| Group 1 | 14 | 172,105 | 116,633 |
| Group 2 | 14 | 198,469 | 9,674 |

### Example 11: A Phase 1a Randomized, Placebo Controlled Study to Evaluate the Monovalent SARS-CoV-2 VLP Vaccine in Human Subjects

An investigational vaccine called VBI-2902a was formulated using 5 μg of the SARS-CoV-2 Proline and Furin Cleavage Modified with TM/Cyt from VSV (SEQ ID NO: 24) spike protein with 0.33 mg/ml aluminum phosphate adjuvant (Adjuphos®). Sixty (60) healthy adults, 18-54 years of age with no previous clinical or laboratory diagnosis of COVID-19 or SARS-CoV-2 infection and not previously vaccinated with an experimental or authorized COVID-19 vaccine were enrolled in Phase 1a clinical study to evaluate the safety, tolerability and immunogenicity of a one-dose or two-dose regimen. Participants were randomized into the following groups:
Group G1—20 participants received VBI-2902a at a dose of 5 μg of spike protein at Day 1 and placebo at Day 28;
Group G2—20 participants received VBI-2902a at a dose of 5 μg of spike protein at

Days 1 and 28; and
Group G3—20 participants received placebo at Days 1 and 28.

All subjects were seronegative at the start of the trial. Immunogenicity (antibody binding and neutralization titers) was measured on Days 0, 7, 28, 35, and 56. Prior to Day 56 there were 11 individuals who chose to be vaccinated with COVID-19 vaccines available under emergency use authorization and there were 3 confirmed cases of SARS-CoV-2 infection (no infections occurred in the two-dose cohort). Immunogenicity results excluded these 14 subjects.

There were no antibody or neutralization responses detected in any subjects in the placebo group at any time point, except for a single subject at Days 35 and 56 which likely represented an undiagnosed case of COVID-19 given the relatively high antibody binding and neutralization titers in this subject.

Immunogenicity assessments utilized validated assays that assessed binding titers against a recombinant SARS-CoV-2 spike protein and neutralization titers based on a pseudovirus neutralization assay, with both assays using a Wuhan SARS-CoV-2 sequence. A panel of 25 COVID-19 convalescent sera used by the Contract Research Organization, Nexelis, during validation of the assay was used a benchmark for vaccine-induced immunogenicity. All 25 individuals had PCR-confirmed COVID-19 infection and mild to moderate symptoms.

In the single dose cohort, seroconversion based on antibody binding titers was observed in 12 of 16 subjects, but the titers were generally modest and significantly below those observed among convalescent sera. Neutralizing antibody responses were detected in only 8 of the 12 subjects that seroconverted and were typically just above the sensitivity of the assay.

Among subjects in the 2-dose cohort, there was clear evidence of boosting in both the antibody binding and neutralization titers 7 days after the second dose (Day 35), and antibody responses continued to increase from Days 28 to 56. By Day 56 all subjects had seroconverted. Immunity in this cohort was robust, with neutralization titers 4.3-fold greater than that of the convalescent sera on Day 56 (GMT of 329 vs. 76).

### Example 12: Evaluation of Potency and Protective Effect of a Monovalent SARS-CoV-2 Beta Variant VLP Vaccine as a Booster

VLPs were produced using the method described Example 2 except that the SARS-CoV-2 spike protein sequence cloned into the Propol II expression vector was a SARS-CoV-2 Beta variant modified with TM/Cyt from VSV (SEQ ID NO:30). The SARS-CoV-2 sequence was from the Beta variant B 1.351 isolate EPI_ISL_911433 (GISAID). Vaccine formulations (referred to as VBI-2905a) were prepared using this Beta variant spike protein (0.1 μg) adjuvanted with aluminum phosphate adjuvant (Adjuphos®) (125 μg). Vaccine formulations referred to as VBI-2902a, described in Example 11, based on the triple modified SARs-CoV-2 Wuhan spike protein (SEQ ID: No. 24) (0.1 μg) adjuvanted with aluminum phosphate adjuvant (125 μg) were also prepared.

Naïve 6-8 week old C57/BL6 mice were immunized with 2 injections of VBI-2902a, 2 injections of VBI-2905a, or a first injection of VBI-2902a followed by a second injection of VBI-2905a (heterologous boost). Blood was collected on day 1 before injection and day 14 after each injection for humoral immunity assessment.

Anti-SARS-CoV-2 specific IgG binding titers in sera were measured by standard ELISA using recombinant SARS-CoV-2 spike RBD proteins (Sinobiological). For total IgG binding titers, detection was performed using a goat anti-mouse IgG-Fc HRP (Bethyl), or Goat anti-Hamster IgG HRP (ThermoFisher), or goat anti-human IgG heavy and light chain HRP-conjugated (Bethyl). HRP-conjugated Goat anti-mouse IgG1 and HRP-conjugated goat anti-mouse IgG2b HRP (Bethyl) were used for the detection of isotype subtype. Determination of antibody binding titers to spike RBDs was performed using a SARS-COV-2 RDB recombinant protein. The detection was completed by adding 3,3′,5,5′-tetramethylbenzidine (TMB) substrate solution, and the reaction stopped by adding liquid stop solution for TMB substrate. Absorbance was read at 450 nm in an ELISA microwell plate reader. Data fitting and analysis were performed with SoftMaxPro 5, using a four-parameter fitting algorithm.

Neutralizing activity in mouse serum samples was measured by standard plaque reduction neutralization test (PRNT) on Vero cells using 100 PFU of SARS-CoV2/Canada/ON/VIDO-01/2020 (Wu-1 virus) or hCoV-19/South Africa/KRISP-EC-K005321/2020 (Beta virus). Results were represented as PRNT90 end point titer (EPT), corresponding to the lowest dilution inhibiting respectively 90% of plaque formation in Vero cell culture.

As shown in Table 39 below, antibody binding titers to spike protein RBDs showed significant benefit of a prime vaccination with VBI-2902a and a boost with VBI-2905a. VBI-2902a induced high levels (most of the sera>106 EPT with GMT 974x103) of antibody binding titers against the ancestral Wuhan spike RBD with significantly reduced cross-reactivity against the Beta variant RBD (GMT 74x103), though there was good cross-reactivity against the Delta variant RBD (GMT 616x103). Antisera from immunization with VBI-2905a showed similar cross-reactivity against the ancestral Wuhan, Delta and Beta RBD (respectively GMT 322x103, 192x103 and 217x103). However, mice receiving the heterologous prime boost regimen showed strong reactivity to each of the ancestral Wuhan and Delta RBD and to Beta RBD.

TABLE 39
Antibody Binding to RBD from Different SARS-CoV-2 Strains

| | | | | | Wuhan-1 IgG | Beta IgG | Delta IgG |
|---|---|---|---|---|---|---|---|
| | Mouse | Wuhan-1 | Beta RBD | Delta RBD | Geometric | Geometric | Geometric |
| Group | # | RBD IgG | IgG | IgG | Mean | Mean | Mean |
| VBI-2902a | 25 | 1,622,653 | 200,759 | 704,003 | 974,453 | 74,510 | 615,553 |
| | 26 | 1,408,501 | 64,036 | 1,236,698 | | | |
| | 27 | 988,821 | 82,088 | 734,939 | | | |
| | 28 | 112,147 | 85,278 | 633,767 | | | |
| | 29 | 2,463,443 | 146,560 | 810,595 | | | |
| | 30 | 1,391,874 | 59,195 | 636,826 | | | |
| | 31 | 1,049,217 | 32,930 | 325,804 | | | |
| | 32 | 891,650 | 36,949 | 302,222 | | | |
| VBI-2905a | 9 | 661,450 | 229,879 | 318,469 | 321,718 | 217,804 | 192,398 |
| | 10 | 555,023 | 201,817 | 287,502 | | | |
| | 11 | 398,020 | 294,635 | 213,003 | | | |
| | 12 | 462,292 | 393,792 | 303,944 | | | |
| | 13 | 460,374 | 400,407 | 237,333 | | | |
| | 14 | 56,080 | 32,686 | 22,659 | | | |
| | 15 | 218,736 | 169,591 | 232,429 | | | |
| | 16 | 300,841 | 423,893 | 253,421 | | | |
| Heterologous | 49 | 405,310 | 71,244 | 317,281 | 1,045,821 | 158,111 | 675,623 |
| Boost | 50 | 976,428 | 260,043 | 1,025,332 | | | |
| | 51 | 233,628 | 36,070 | 234,033 | | | |
| | 52 | 1,872,244 | 606,640 | 780,546 | | | |
| | 69 | 2,325,516 | 163,278 | 1,839,676 | | | |
| | 70 | 1,270,431 | 210,707 | 517,830 | | | |
| | 71 | 2,061,624 | 197,265 | 884,471 | | | |
| | 72 | 1,357,256 | 141,962 | 867,044 | | | |

Neutralizing data is shown in Table 40 below. Two doses of VBI-2902a induced high levels of neutralizing antibody response against the ancestral Wuhan strain of SARS-CoV-2 (GMT=2,458) but significantly lower activity against the Beta variant (GMT=94). By contrast, VBI-2905a induced antibodies that neutralized Beta and ancestral Wuhan viruses at similar levels in mice, yielding only a 2.2-fold difference with non significant p=0.1484. Sera from mice in the heterologous boost group cross-neutralized both the Beta variant and the ancestral Wuhan strain with similar potencies (1,4 fold difference with p=0.3828). Heterologous boosting with VBI-2905a significantly increased the PRNT90 against the ancestral strain compared to 2 doses of VBI-2905a alone (from GMT of 371 to 820, p=0.0267) to levels that were closer to those reached after two doses of VBI-2902a.

TABLE 40
Neutralizing Antibodies Against Wuhan and Beta Strains of SARS-Cov-2

| | | Wuhan-1 | Beta | | |
|---|---|---|---|---|---|
| | | (hCoV-19/ | (hCoV-19/ | Wuhan-1 | Beta |
| | | Canada/ | South Africa/ | PRNT90 | PRNT90 |
| | Mouse | ON-VIDO- | KRISP-EC- | Geometric | Geometric |
| Group | # | 01/2020) | K005321/2020) | Mean | Mean |
| VBI-2902a | 25 | 5,120 | 136 | 2,458 | 94 |
| | 26 | 3,414 | 20 | | |
| | 27 | 853 | 176 | | |
| | 28 | 3,149 | 99 | | |
| | 29 | 5,120 | 320 | | |
| | 30 | 3,149 | 226 | | |
| | 31 | 344 | 40 | | |
| | 32 | 5,120 | 43 | | |
| VBI-2905a | 9 | 640 | 693 | 371 | 619 |
| | 10 | 320 | 1,907 | | |
| | 11 | 800 | 2,560 | | |
| | 12 | 452 | 1,483 | | |
| | 13 | 427 | 349 | | |
| | 14 | 40 | 182 | | |
| | 15 | 686 | 789 | | |
| | 16 | 413 | 86 | | |
| Heterologous | 49 | 723 | 542 | 820 | 564 |
| Boost | 50 | 827 | 904 | | |
| | 51 | 320 | 343 | | |
| | 52 | 1,721 | 827 | | |
| | 69 | 751 | 452 | | |
| | 70 | 640 | 1,653 | | |
| | 71 | 827 | 91 | | |
| | 72 | 1,566 | 1,089 | | |

In order to evaluate the protective effect of a heterologous boosting strategy with VBI-2905a, a challenge study was conducted in golden hamsters. Golden Syrian hamsters were intramuscularly vaccinated 3 weeks apart with two doses VBI-2902a, two doses of VBI-2905a or a priming dose of VBI-2902a followed by a second, booster dose of VBI-2905a, each formulation as described above.

Three weeks after the second immunization, hamsters were exposed to 1×105 TCID₅₀ of the Beta variant virus in each nare. In the placebo group, hamsters began losing weight the day after infection which continued until day 6-8. Vaccination with 2 doses of VBI-2902a based on the ancestral Wuhan spike protein induced limited protection against challenge with the Beta variant with moderate weight loss recorded until day 4, and only a fraction (⅗) of the animals fully regained their initial body weight after day 7. By contrast, hamsters vaccinated with 2 doses of VBI-2905a exhibited transient weight loss up to day 2-3 and then rapidly regained weight. A similar pattern was observed in hamsters that received VBI-2905a as a boost indicating that the hamsters received the same level of protection against the Beta variant from a single boost as from a two-dose regime.

What is claimed is:

1. An immunogenic composition comprising a virus-like particle (VLP) comprising:
a first polypeptide that is a gag protein found in murine leukemia virus (MLV) having at least 95% identity with the amino acid sequence of SEQ ID NO:1;
at least one additional polypeptide which is a spike glycoprotein from the Beta variant of the SARS-CoV-2 coronavirus; and
a pharmaceutically acceptable carrier.

2. The immunogenic composition of claim 1 wherein the spike glycoprotein is a modified protein.

3. The immunogenic composition of claim 2 wherein the modified protein has a deletion at a furin cleavage site.

4. The immunogenic composition of claim 2 wherein the modified protein has a transmembrane domain from VSV.

5. The immunogenic composition of claim 2 wherein the modified protein has a lysine residue and a valine residue replaced with proline residues.

6. The immunogenic composition of claim 5 wherein the modified protein has a transmembrane domain from VSV.

7. The immunogenic composition of claim 2 wherein the modified protein has a lysine residue and a valine residue replaced with proline residues and has a deletion at a furin cleavage site.

8. The immunogenic composition of claim 2 wherein the modified protein has a lysine residue and a valine residue replaced with proline residues and has a deletion at a furin cleavage site and has a transmembrane domain from VSV.

9. The immunogenic composition of claim 1, wherein the additional polypeptide has an amino acid sequence of SEQ ID NO: 28.

10. The immunogenic composition of claim 1, further comprising an adjuvant.

11. The immunogenic composition of claim 10, wherein the adjuvant is selected from the group consisting of cytokines, gel-type adjuvants, microbial adjuvants, oil-emulsion and emulsifier-based adjuvants, particulate adjuvants, synthetic adjuvants, polymer adjuvants, and/or combinations thereof.

12. The immunogenic composition of claim 11, wherein the particulate adjuvant is an aluminum salt.

13. The immunogenic composition of claim 1, wherein the VLP is produced by co-transfecting a host cell with a first vector comprising a nucleotide sequence of SEQ ID NO: 3 and a second vector comprising a nucleotide sequence of SEQ ID NO: 30; and
cultivating the host cell in a suitable medium under conditions allowing the expression of the proteins encoded by the vectors.

14. A method of treating a subject having or at risk for coronavirus infection, comprising administering to the subject the pharmaceutical composition of claim 1.

15. The method of claim 14 wherein the pharmaceutical composition of claim 1 is administered as a booster following vaccination with a different vaccine against SARS-CoV-2.
